SEQUENCE LISTING (8 sequences)

SEQ ID NO: 1 - DNA, 2079 bp, Flavivirus ZIKV
atgcggagaggagcagacacatccgtgggaatcgtgggcctgctgctgaccacagcaatggcagccgaggtgaccaggagaggcagcgcctactatatgtacctggacagaaatgatgccggcgaggccatctcctttcccaccacactgggcatgaacaagtgctacatccagatcatggacctgggccacatgtgcgatgccaccatgagctatgagtgtccaatgctggacgagggcgtggagcccgacgatgtggattgctggtgtaataccacatctacatgggtggtgtacggcacctgtcaccacaagaagggagaggcacggcgcagcaggagagcagtgacactgccttcccactctaccaggaagctgcagacaagaagccagacctggctggagtccagggagtatacaaagcacctgatcagggtggagaactggatctttagaaatccaggattcgcactggctgccgccgccatcgcatggctgctgggcagctccaccagccagaaagtgatctacctggtcatgatcctgctgatcgcccctgcctattctatccggtgcatcggcgtgagcaatagggacttcgtggagggaatgtccggaggcacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactccccacgcgccgaggccaccctgggaggatttggctctctgggcctggactgcgagcctaggacaggcctggacttctccgatctgtactatctgaccatgaacaataagcactggctggtgcacaaggagtggtttcacgacatcccactgccatggcacgcaggagcagatacaggcaccccacactggaacaataaggaggccctggtggagttcaaggacgcccacgccaagcggcagacagtggtggtgctgggcagccaggagggagcagtgcacaccgccctggcaggcgccctggaggccgagatggacggagcaaagggccgcctgtctagcggccacctgaagtgcaggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggacccccctttcggcgatagctatatcgtgatcggcgtgggcgagaagaagatcacacaccactggcacagaagcggctccacaatcggcaaggcctttgaggcaaccgtgcggggagcaaagaggatggccgtgctgggcgacaccgcatgggatttcggatctgtgggaggcgccctgaacagcctgggcaagggcatccaccagatcttcggcgccgcctttaagtccctgttcggcggcatgagctggttttcccagatcctgatcggcacactgctgatgtggctgggcctgaacaccaagaatggctctatcagcctgatgtgcctggccctgggaggcgtgctgatcttcctgtccaccgccgtgtctgcc

SEQ ID NO: 2 - DNA, 12580 bp, Artificial Sequence
  DNA plasmid construct
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgcggagaggagcagacacatccgtgggaatcgtgggcctgctgctgaccacagcaatggcagccgaggtgaccaggagaggcagcgcctactatatgtacctggacagaaatgatgccggcgaggccatctcctttcccaccacactgggcatgaacaagtgctacatccagatcatggacctgggccacatgtgcgatgccaccatgagctatgagtgtccaatgctggacgagggcgtggagcccgacgatgtggattgctggtgtaataccacatctacatgggtggtgtacggcacctgtcaccacaagaagggagaggcacggcgcagcaggagagcagtgacactgccttcccactctaccaggaagctgcagacaagaagccagacctggctggagtccagggagtatacaaagcacctgatcagggtggagaactggatctttagaaatccaggattcgcactggctgccgccgccatcgcatggctgctgggcagctccaccagccagaaagtgatctacctggtcatgatcctgctgatcgcccctgcctattctatccggtgcatcggcgtgagcaatagggacttcgtggagggaatgtccggaggcacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactccccacgcgccgaggccaccctgggaggatttggctctctgggcctggactgcgagcctaggacaggcctggacttctccgatctgtactatctgaccatgaacaataagcactggctggtgcacaaggagtggtttcacgacatcccactgccatggcacgcaggagcagatacaggcaccccacactggaacaataaggaggccctggtggagttcaaggacgcccacgccaagcggcagacagtggtggtgctgggcagccaggagggagcagtgcacaccgccctggcaggcgccctggaggccgagatggacggagcaaagggccgcctgtctagcggccacctgaagtgcaggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggacccccctttcggcgatagctatatcgtgatcggcgtgggcgagaagaagatcacacaccactggcacagaagcggctccacaatcggcaaggcctttgaggcaaccgtgcggggagcaaagaggatggccgtgctgggcgacaccgcatgggatttcggatctgtgggaggcgccctgaacagcctgggcaagggcatccaccagatcttcggcgccgcctttaagtccctgttcggcggcatgagctggttttcccagatcctgatcggcacactgctgatgtggctgggcctgaacaccaagaatggctctatcagcctgatgtgcctggccctgggaggcgtgctgatcttcctgtccaccgccgtgtctgcctgaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaagcggccgcgagcttggctcgagcctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgttaatacgactcactatag

SEQ ID NO: 3 - DNA, 12 bp, Artificial Sequence
  optimized subgenomic promoter
tagtccgccaag

SEQ ID NO: 4 - DNA, 11139 bp, Artificial Sequence
  DNA plasmid construct
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgctcctcttgctccttctcttgggactccgattgcagctctctttgggtataattcctgtcgaagaagagaatcccgacttctggaatcgcgaagcggcagaagcattgggtgcggcaaaaaagctccaaccggcacagactgcggccaagaatctcataattttcttgggtgatggcatgggcgtttccactgtgactgcggcacgaatcctgaagggtcaaaagaaggataaactgggtccagagatcccgctggctatggataggtttccatacgttgcgctctctaagacctataacgtcgataagcatgtaccagactccggagcgacggccactgcttacctttgtggagttaaaggtaatttccaaacaataggactgagcgcagctgcaagattcaaccaatgcaacacgacaagagggaatgaggtgatttcagtcatgaatcgagcaaagaaagctgggaaatccgttggcgtggtcaccactacaagagtgcagcatgcatctccagcaggaacttacgcccacactgtaaacagaaactggtatagtgacgccgatgttcccgcctctgcgaggcaagagggttgtcaagacatcgctacccagctcataagcaacatggacattgatgtaatactgggtgggggtcggaaatacatgttccggatgggcactcccgatccggagtacccggatgactattcccaaggaggtaccagattggacgggaaaaatcttgtccaagaatggctcgccaagcggcagggggcaaggtacgtgtggaacaggacagaactgatgcaggcaagcttggatccaagcgtaacgcatcttatgggtctttttgaacccggtgatatgaaatacgaaatacatcgcgactcaacactggacccgtctctcatggagatgactgaagctgccttgaggttgttgagtcggaaccctaggggctttttcttgttcgtagagggcgggcgaattgaccacggtcatcacgaatctcgagcgtaccgggcgctcacagaaaccatcatgtttgacgatgctatcgaacgagcgggtcagcttacctctgaagaagatacgctctctcttgtcaccgcggaccatagccatgttttttccttcggtggttatccgttgcgaggttccagcatattcggcctcgcgccagggaaagcccgcgaccgcaaagcttatacggtgctgctttacggaaacggccctggttacgtccttaaagacggtgcgagacctgacgtgacggaatctgaatccggttctcccgaatatagacaacagagtgctgttccgctggatgaagagactcatgcgggagaagatgtagcggtttttgctagggggccgcaagcacaccttgttcatggcgttcaggagcaaactttcatagcccatgtaatggcatttgctgcgtgtctcgagccgtataccgcttgcgatctcgctccgccggcgggtacaaccgatgctgcccacccggggtactcaagagtaggggcagcagggcgatttgaacaaacttgataaggcgcgccatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaagcggccgctcgagcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgacgcgttaatacgactcactatag

SEQ ID NO: 5 - DNA, 12056 bp, Artificial Sequence
  DNA plasmid construct
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgctcctcttgctccttctcttgggactccgattgcagctctctttgggtataattcctgtcgaagaagagaatcccgacttctggaatcgcgaagcggcagaagcattgggtgcggcaaaaaagctccaaccggcacagactgcggccaagaatctcataattttcttgggtgatggcatgggcgtttccactgtgactgcggcacgaatcctgaagggtcaaaagaaggataaactgggtccagagatcccgctggctatggataggtttccatacgttgcgctctctaagacctataacgtcgataagcatgtaccagactccggagcgacggccactgcttacctttgtggagttaaaggtaatttccaaacaataggactgagcgcagctgcaagattcaaccaatgcaacacgacaagagggaatgaggtgatttcagtcatgaatcgagcaaagaaagctgggaaatccgttggcgtggtcaccactacaagagtgcagcatgcatctccagcaggaacttacgcccacactgtaaacagaaactggtatagtgacgccgatgttcccgcctctgcgaggcaagagggttgtcaagacatcgctacccagctcataagcaacatggacattgatgtaatactgggtgggggtcggaaatacatgttccggatgggcactcccgatccggagtacccggatgactattcccaaggaggtaccagattggacgggaaaaatcttgtccaagaatggctcgccaagcggcagggggcaaggtacgtgtggaacaggacagaactgatgcaggcaagcttggatccaagcgtaacgcatcttatgggtctttttgaacccggtgatatgaaatacgaaatacatcgcgactcaacactggacccgtctctcatggagatgactgaagctgccttgaggttgttgagtcggaaccctaggggctttttcttgttcgtagagggcgggcgaattgaccacggtcatcacgaatctcgagcgtaccgggcgctcacagaaaccatcatgtttgacgatgctatcgaacgagcgggtcagcttacctctgaagaagatacgctctctcttgtcaccgcggaccatagccatgttttttccttcggtggttatccgttgcgaggttccagcatattcggcctcgcgccagggaaagcccgcgaccgcaaagcttatacggtgctgctttacggaaacggccctggttacgtccttaaagacggtgcgagacctgacgtgacggaatctgaatccggttctcccgaatatagacaacagagtgctgttccgctggatgaagagactcatgcgggagaagatgtagcggtttttgctagggggccgcaagcacaccttgttcatggcgttcaggagcaaactttcatagcccatgtaatggcatttgctgcgtgtctcgagccgtataccgcttgcgatctcgctccgccggcgggtacaaccgatgctgcccacccggggtactcaagagtaggggcagcagggcgatttgaacaaacttgataataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaagcggccgcgagcttggctcgagcctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgttaatacgactcactatag

SEQ ID NO: 6 - DNA, 14350 bp, Artificial Sequence
  DNA plasmid construct
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcctgctgaccaccaaacgcaccatgttcgtgttcctggttcttctgcctctggtgtctagccagtgtgtgaatctgaccacaaggacccaacttcctcctgcctacacaaacagcttcaccagaggcgtgtactaccctgataaggtgttccggtcctcagtgttgcatagcacgcaggacctctttctgcccttcttcagcaacgtgacctggttccacgccatccatgtgtctggcaccaatggcaccaagagattcgacaatcccgttctgcccttcaatgatggcgtgtactttgccagcaccgagaagagcaacatcatccggggatggatttttggtactactttagatagcaagacacagtctctgctgatcgtgaacaatgccaccaacgtggtgattaaggtgtgcgagttccagttctgcaacgacccctttctgggcgtgtattaccacaagaacaacaagtcctggatggagagcgagttccgggtgtatagttcagcaaacaattgcacattcgaatatgtttctcagcctttcctgatggacctggagggcaaacagggcaattttaaaaacttacgggagtttgtgttcaagaacatcgacggctattttaagatctactcaaaacacactcctataaacctggtgagggacctgcctcagggcttctcagccctagagcctctcgtcgatctccctatcggcatcaacatcacccggttccagaccctgttagctctgcacagaagctatctgacacctggcgattcttcttctggatggacagctggagctgccgcctattatgtgggctatttacagcctagaaccttcctgttgaagtacaacgagaatggcaccatcaccgacgctgtggattgtgctcttgatcctctgtctgagaccaagtgtaccctgaagagcttcacagtggagaagggcatctaccagaccagcaacttcagagtgcagcctacagagagcatcgtgagattccccaacatcaccaacctgtgcccatttggcgaggtgtttaatgccaccagattcgcatcagtgtacgcatggaacagaaagaggatcagcaattgcgtggccgattatagcgtgttgtacaattcagcttcgtttagcacgttcaagtgttatggcgtatcccctaccaagctgaatgacctgtgcttcacaaacgtctacgctgacagcttcgtgattagaggcgatgaggtgagacagattgctcctggacaaacaggcaagattgccgactacaactacaagctgcccgacgactttaccggctgtgtgattgcctggaattctaataaccttgatagtaaagtgggagggaattacaattatctctaccggcttttccggaagagcaacctgaagccattcgagagagatatcagcaccgagatctatcaggctggcagcacaccctgtaatggagtggagggcttcaactgctactttcctctgcaaagctatggctttcaacccacaaacggagtgggatatcagccctacagagtggttgttctgagcttcgaactgctgcatgctcctgctacagtgtgtggccctaaaaagagtactaatctggtcaaaaataagtgcgtgaacttcaatttcaatggcctgaccggcacaggagttctgacagagagcaacaaaaagttcctccctttccagcagtttggaagggatatcgccgacaccacagatgccgtgagagatcctcaaacactggagatcctggacattaccccttgctcttttggaggcgtgagcgtgatcacacctggcacaaataccagcaatcaggtggctgtgctgtatcagggagtgaattgcaccgaggttccagtggccattcatgctgatcaactgacccctacctggagagtgtacagcacaggctctaacgtgtttcagaccagagctggatgtctgattggagccgaacacgtgaacaacagctacgagtgcgatatccctattggagccggcatttgtgcctcttaccagacacagaccaatagccccagaagagccagatctgtggcttctcagagcattatcgcctacaccatgtctctgggagccgagaattctgtggcctacagcaacaactctatcgccatccctaccaacttcaccatcagcgtgaccaccgagattctgcctgtgagcatgacaaagacaagcgtggattgcaccatgtacatctgcggcgatagcaccgagtgcagcaatctgctgttacagtacggaagtttttgtacccagctgaatagagccctgacaggcattgccgtggaacaggacaagaacacacaggaggtgtttgctcaggtgaaacagatctacaagactccccctataaaggactttggcggcttcaacttcagccagattctgcctgatccttctaagcctagcaagcggagcttcatcgaagacctgctgttcaacaaggtgacactggccgatgccggctttattaagcagtacggcgattgtctgggcgatatcgctgccagagatctgatttgcgcccagaaattcaatggtctaacagtgcttcctcctctgctgacagatgagatgattgcccagtacacaagcgctctgttagccggcacaattacatctggatggacatttggagctggagctgctctgcaaattccttttgccatgcagatggcctacagattcaatgggatcggagtgacccagaacgtgctgtacgagaaccagaagctcatagccaaccagttcaattctgccatcggcaagatccaggacagcctgagctctacagcttctgctctgggcaaactgcaggatgttgtgaatcagaatgcgcaggctttaaacactctggtgaaacagctgagcagcaattttggcgccatcagctctgtgcttaatgacatcctgagcaggctggaccctcctgaagctgaagtgcaaatcgaccggctcatcaccgggcgcctgcagtctctgcagacatacgtcactcagcaactgatcagagctgccgagattcgcgcgagtgccaatctggctgccaccaagatgtctgagtgtgttctggggcaatcaaagcgcgtggatttctgcggcaagggatatcacctgatgagcttccctcagtctgctcctcatggagtggtgttcctgcatgtgacctatgtgcctgctcaggagaagaatttcacaacagcccctgccatctgccacgatggaaaagcccactttccaagagaaggcgtgttcgtgtctaatggaacacactggttcgtgacccagcggaacttctacgaaccccagatcatcaccaccgacaacacatttgtgagcggcaattgcgatgtggtgatcggcatcgtgaacaacaccgtgtacgaccctctgcaacctgaactggacagctttaaggaggagctggacaagtactttaagaaccatacgagccctgacgtggatctgggcgacatcagtggtatcaatgctagcgtggtgaatatccagaaggagatcgaccggctgaatgaagtggccaagaacctgaacgaaagcctgatcgacctgcaagaactgggcaagtatgagcagtacatcaagtggccctggtacatctggctgggctttattgccggactgatcgccatcgttatggtgaccattatgctgtgctgcatgaccagctgctgctcttgtctgaagggctgttgctcttgtggctcttgctgtaagttcgatgaggacgattccgagcctgtcctcaagggggtcaaactccactacacctgatgaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaagcggccgcgagcttggctcgagcctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgttaatacgactcactatag

SEQ ID NO: 7 - DNA, 3852 bp, SARS-CoV-2
atgttcctgctgaccaccaaacgcaccatgttcgtgttcctggttcttctgcctctggtgtctagccagtgtgtgaatctgaccacaaggacccaacttcctcctgcctacacaaacagcttcaccagaggcgtgtactaccctgataaggtgttccggtcctcagtgttgcatagcacgcaggacctctttctgcccttcttcagcaacgtgacctggttccacgccatccatgtgtctggcaccaatggcaccaagagattcgacaatcccgttctgcccttcaatgatggcgtgtactttgccagcaccgagaagagcaacatcatccggggatggatttttggtactactttagatagcaagacacagtctctgctgatcgtgaacaatgccaccaacgtggtgattaaggtgtgcgagttccagttctgcaacgacccctttctgggcgtgtattaccacaagaacaacaagtcctggatggagagcgagttccgggtgtatagttcagcaaacaattgcacattcgaatatgtttctcagcctttcctgatggacctggagggcaaacagggcaattttaaaaacttacgggagtttgtgttcaagaacatcgacggctattttaagatctactcaaaacacactcctataaacctggtgagggacctgcctcagggcttctcagccctagagcctctcgtcgatctccctatcggcatcaacatcacccggttccagaccctgttagctctgcacagaagctatctgacacctggcgattcttcttctggatggacagctggagctgccgcctattatgtgggctatttacagcctagaaccttcctgttgaagtacaacgagaatggcaccatcaccgacgctgtggattgtgctcttgatcctctgtctgagaccaagtgtaccctgaagagcttcacagtggagaagggcatctaccagaccagcaacttcagagtgcagcctacagagagcatcgtgagattccccaacatcaccaacctgtgcccatttggcgaggtgtttaatgccaccagattcgcatcagtgtacgcatggaacagaaagaggatcagcaattgcgtggccgattatagcgtgttgtacaattcagcttcgtttagcacgttcaagtgttatggcgtatcccctaccaagctgaatgacctgtgcttcacaaacgtctacgctgacagcttcgtgattagaggcgatgaggtgagacagattgctcctggacaaacaggcaagattgccgactacaactacaagctgcccgacgactttaccggctgtgtgattgcctggaattctaataaccttgatagtaaagtgggagggaattacaattatctctaccggcttttccggaagagcaacctgaagccattcgagagagatatcagcaccgagatctatcaggctggcagcacaccctgtaatggagtggagggcttcaactgctactttcctctgcaaagctatggctttcaacccacaaacggagtgggatatcagccctacagagtggttgttctgagcttcgaactgctgcatgctcctgctacagtgtgtggccctaaaaagagtactaatctggtcaaaaataagtgcgtgaacttcaatttcaatggcctgaccggcacaggagttctgacagagagcaacaaaaagttcctccctttccagcagtttggaagggatatcgccgacaccacagatgccgtgagagatcctcaaacactggagatcctggacattaccccttgctcttttggaggcgtgagcgtgatcacacctggcacaaataccagcaatcaggtggctgtgctgtatcagggagtgaattgcaccgaggttccagtggccattcatgctgatcaactgacccctacctggagagtgtacagcacaggctctaacgtgtttcagaccagagctggatgtctgattggagccgaacacgtgaacaacagctacgagtgcgatatccctattggagccggcatttgtgcctcttaccagacacagaccaatagccccagaagagccagatctgtggcttctcagagcattatcgcctacaccatgtctctgggagccgagaattctgtggcctacagcaacaactctatcgccatccctaccaacttcaccatcagcgtgaccaccgagattctgcctgtgagcatgacaaagacaagcgtggattgcaccatgtacatctgcggcgatagcaccgagtgcagcaatctgctgttacagtacggaagtttttgtacccagctgaatagagccctgacaggcattgccgtggaacaggacaagaacacacaggaggtgtttgctcaggtgaaacagatctacaagactccccctataaaggactttggcggcttcaacttcagccagattctgcctgatccttctaagcctagcaagcggagcttcatcgaagacctgctgttcaacaaggtgacactggccgatgccggctttattaagcagtacggcgattgtctgggcgatatcgctgccagagatctgatttgcgcccagaaattcaatggtctaacagtgcttcctcctctgctgacagatgagatgattgcccagtacacaagcgctctgttagccggcacaattacatctggatggacatttggagctggagctgctctgcaaattccttttgccatgcagatggcctacagattcaatgggatcggagtgacccagaacgtgctgtacgagaaccagaagctcatagccaaccagttcaattctgccatcggcaagatccaggacagcctgagctctacagcttctgctctgggcaaactgcaggatgttgtgaatcagaatgcgcaggctttaaacactctggtgaaacagctgagcagcaattttggcgccatcagctctgtgcttaatgacatcctgagcaggctggaccctcctgaagctgaagtgcaaatcgaccggctcatcaccgggcgcctgcagtctctgcagacatacgtcactcagcaactgatcagagctgccgagattcgcgcgagtgccaatctggctgccaccaagatgtctgagtgtgttctggggcaatcaaagcgcgtggatttctgcggcaagggatatcacctgatgagcttccctcagtctgctcctcatggagtggtgttcctgcatgtgacctatgtgcctgctcaggagaagaatttcacaacagcccctgccatctgccacgatggaaaagcccactttccaagagaaggcgtgttcgtgtctaatggaacacactggttcgtgacccagcggaacttctacgaaccccagatcatcaccaccgacaacacatttgtgagcggcaattgcgatgtggtgatcggcatcgtgaacaacaccgtgtacgaccctctgcaacctgaactggacagctttaaggaggagctggacaagtactttaagaaccatacgagccctgacgtggatctgggcgacatcagtggtatcaatgctagcgtggtgaatatccagaaggagatcgaccggctgaatgaagtggccaagaacctgaacgaaagcctgatcgacctgcaagaactgggcaagtatgagcagtacatcaagtggccctggtacatctggctgggctttattgccggactgatcgccatcgttatggtgaccattatgctgtgctgcatgaccagctgctgctcttgtctgaagggctgttgctcttgtggctcttgctgtaagttcgatgaggacgattccgagcctgtcctcaagggggtcaaactccactacacctgatga

SEQ ID NO: 8 - protein, 9 aa, SARS-CoV-2
MFLLTTKRT

(19) United States
(12) Patent Application Publication
(10) Pub. No.: US 2023/0310323 A1
(43) Pub. Date: Oct. 5, 2023

(54) CO-LYOPHILIZED RNA AND NANOSTRUCTURED LIPID CARRIER

(71) Applicant: ACCESS TO ADVANCED HEALTH INSTITUTE, Seattle, WA (US)

(72) Inventors: Ryan M. KRAMER, Seattle, WA (US); Emily VOIGHT, Seattle, WA (US); Alana GERHARDT, Seattle, WA (US); Michelle ARCHER, Seattle, WA (US)

(21) Appl. No.: 18/024,733

(22) PCT Filed: Jul. 2, 2021
(86) PCT No.: PCT/US21/40388

§ 371 Date: Mar. 3, 2023

Related U.S. Application Data
(60) Provisional application No. 63/075,032, filed on Sep. 4, 2020.
(60) Provisional application No. 63/107,383, filed on Oct. 29, 2020.
(60) Provisional application No. 63/144,169, filed on Feb. 1, 2021.

(51) Int. Cl. A61K 9/19; A61K 9/107; A61K 39/12

(52) U.S. Cl. CPC A61K 9/19; A61K 9/1075; A61K 39/12; A61K 2039/55555

(57) ABSTRACT

This disclosure provides thermostable, lyophilized compositions of nano structured lipid carrier (NLC) particles, methods of making the compositions, and methods of using the compositions for stimulating an immune response. The lyophilized compositions are in the form of cakes that form oil-in-water emulsions upon reconstitution. The compositions comprise NLC particles lyophilized in the presence of a cake-forming excipient. The compositions may be lyophilized with a bioactive agent, or the bioactive agent may be added after reconstitution. The bioactive agent may be RNA that encodes an antigen such as a viral protein. The thermostable, lyophilized compositions have uses as vaccine platforms or vaccines. The lyophilized cake maintains shape, structure, and color for at least 21 months stored at room temperature. Integrity and activity of the bioactive agent is maintained for at least eight months at room temperature and at least 21 months refrigerated.

### CROSS REFERENCE TO RELATED APPLICATIONS

This application claims to priority to U.S. Provisional Application No. 63/075,032 entitled “Co-Lyophilized RNA and Nanostructured Lipid Carrier,” filed on Sep. 4, 2020; U.S. Provisional Application No. 63/107,383 entitled “Co-Lyophilized RNA and Nanostructured Lipid Carrier,” filed on Oct. 29, 2020; and U.S. Provisional Application No. 63/144,169, entitled “A Thermostable, Flexible RNA Vaccine Delivery Platform For Pandemic Response,” filed on Feb. 1, 2021, the disclosures of which are incorporated herein by reference in their entireties.

### STATEMENT REGARDING FEDERALLY SPONSORED RESEARCH OR DEVELOPMENT

This invention was made with government support under Contract No. 75N93019C00059 awarded by National Institute of Allergy and Infectious Diseases, National Institutes of Health, and Department of Health and Human Services and under cooperative agreement HR0011-18-2-0001 from the Defense Advanced Research Projects Agency. The government has certain rights in the invention.

### SEQUENCE LISTING

The Sequence Listing associated with this application is provided in text format in lieu of a paper copy and is hereby incorporated by reference into the specification. The name of the text file containing the Sequence Listing is 56.PCT Sequence Listing_ST25.txt. The text file is 73 KB, was created on Jul. 1, 2021 and is being submitted electronically concurrent with the filing of the specification.

### FIELD

The present disclosure relates generally to the fields of pharmaceutical and vaccine formulations.

### BACKGROUND

RNA-based vaccines show great promise to effectively address existing and emerging infectious diseases (R. P. Deering et al., Nucleic acid vaccines: prospects for non-viral delivery of mRNA vaccines. Expert Opin Drug Deliv 11, 885-899 (2014); S. Rauch et al., New Vaccine Technologies to Combat Outbreak Situations. Front Immunol 9, 1963 (2018); C. Zhang et al., Advances in mRNA Vaccines for Infectious Diseases. Front Immunol 10, 594 (2019)), including the pandemic caused by the SARS-CoV-2 virus. RNA vaccines can be rapidly adapted to new targets and manufactured using sequence-independent operations, thus reducing the cost and time to develop vaccines that target new pathogens (N. Pardi et al., mRNA vaccines—a new era in vaccinology. Nature Reviews Drug Discovery 17, 261-279 (2018)).

However, one of the biggest challenges facing these extraordinary new vaccines is the ability to successfully distribute them widely and rapidly. Strict cold chain requirements for current RNA vaccine formulations greatly complicate global distribution and increase cost. Cold chain storage (−70° C. or −20° C.) is required for RNA vaccines such as the SARS-CoV-2 mRNA vaccines produced by Pfizer/BioNtech and Moderna. Frozen shipping and storage at standard freezer conditions poses difficulties even in settings with well-established medical infrastructure. Maintaining a deep cold chain is much more difficult in areas with limited resources (O. S. Kumru et al., Vaccine instability in the cold chain: mechanisms, analysis and formulation strategies. Biologicals 42, 237-259 (2014); D. Chen and D. Zehrung, Desirable attributes of vaccines for deployment in low-resource settings. J Pharm Sci 102, 29-33 (2013); D. J. A. Crommelin et al., Addressing the Cold Reality of mRNA Vaccine Stability. Journal of Pharmaceutical Sciences, (2020)).

Lack of stability in RNA vaccines is a critical issue, but the physiochemical reasons behind this are under-studied and poorly understood (D. J. A. Crommelin supra). However, several challenges are clear. First, vaccine RNA molecules are prone to cleavage by ubiquitous ribonucleases (i.e., RNAses). Engineering of the RNA molecule itself has previously been done in order to stabilize it (U. Sahin et al., mRNA-based therapeutics—developing a new class of drugs. Nat Rev Drug Discov 13, 759-780 (2014)), but stability problems remain. Second, due to its size, negative charge, and hydrophilicity, RNA alone cannot easily cross a cell membrane to enter target cells upon injection (K. A. Whitehead et al., Knocking down barriers: advances in siRNA delivery. Nat Rev Drug Discov 8, 129-138 (2009)). Thus, RNA delivery formulations are needed to stabilize and protect RNA molecules from degradation (P. S. Kowalski et al., Delivering the Messenger: Advances in Technologies for Therapeutic mRNA Delivery. Mol Ther 27, 710-728 (2019); S. Guan and J. Rosenecker, Nanotechnologies in delivery of mRNA therapeutics using nonviral vector-based delivery systems. Gene Ther 24, 133-143 (2017)).

The current system of choice for delivering RNA vaccines, including all SARS-CoV-2 vaccines in clinical trials to date, is a lipid nanoparticle (LNP) delivery system (L. A. Jackson et al., An mRNA Vaccine against SARS-CoV-2—Preliminary Report. N Engl J Med 383, 1920-1931 (2020); Y. Y. Tam, S. Chen, P. R. Cullis, Advances in Lipid Nanoparticles for siRNA Delivery. Pharmaceutics 5, 498-507 (2013); Y. Zhao and L. Huang, Lipid nanoparticles for gene delivery. Adv Genet 88, 13-36 (2014); A. M. Reichmuth et al., mRNA vaccine delivery using lipid nanoparticles. Therapeutic Delivery 7, 319-334 (2016); K. Bahl et al., Preclinical and Clinical Demonstration of Immunogenicity by mRNA Vaccines against H10N8 and H7N9 Influenza Viruses. Mol Ther 25, 1316-1327 (2017)) in which the negatively-charged RNA molecule is encapsulated within a multicomponent lipid system. This results in 70-100 nm diameter RNA/LNP complexes which protect the RNA from RNase degradation and allow for successful endocytosis by the cell (A. M. Reichmuth et al., mRNA vaccine delivery using lipid nanoparticles. Ther Deliv 7, 319-334 (2016); K. J. Hassett et al., Optimization of Lipid Nanoparticles for Intramuscular Administration of mRNA Vaccines. Mol Ther Nucleic Acids 15, 1-11 (2019)). However, stability of both the RNA and LNP remain an issue (D. J. A. Crommelin supra), with sensitivity to frozen temperatures resulting in major impacts to their colloidal stability after freeze/thaw (R. L. Ball et al., Achieving long-term stability of lipid nanoparticles: examining the effect of pH, temperature, and lyophilization. Int J Nanomedicine 12, 305-315 (2017); P. Zhao et al., Long-term storage of lipid-like nanoparticles for mRNA delivery. Bioact Mater 5, 358-363 (2020)).

A number of alternative lipid-based delivery systems have been proposed and developed to deliver RNA vaccines (L. A. Brito et al., A cationic nanoemulsion for the delivery of next-generation RNA vaccines. Mol Ther 22, 2118-2129 (2014); J. H. Erasmus et al., A Nanostructured Lipid Carrier for Delivery of a Replicating Viral RNA Provides Single, Low-Dose Protection against Zika. Mol Ther 26, 2507-2522 (2018); A. K. Blakney et al., Inside out: optimization of lipid nanoparticle formulations for exterior complexation and in vivo delivery of saRNA. Gene Ther 26, 363-372 (2019)). However, a critical need remains for an effective, thermostable vaccine platform for the delivery of bioactive agents such as RNA that can be distributed without maintaining a cold chain (D. J. A. Crommelin supra) while retaining the ability to elicit an immune response against the vaccine antigen. The present disclosure fulfills these needs and offers other related advantages.

### BRIEF SUMMARY

The present inventors have identified that nanostructured lipid carrier (NLC) particles may be successfully lyophilized in the presence of a cake-forming excipient. This provides a safe and effective NLC-based vaccine delivery system with greatly increased thermostability over current LNP formulations. The vaccine platform may be flexibly adapted for use with a range of bioactive agents. One bioactive agent that may be combined with the NLC particles is RNA such as mRNA or self-amplifying (saRNA). The present inventors have also shown that RNA is protected by co-lyophilization with NLC particles and retains biochemical properties such as the ability to induce protein expression in vivo after at least eight months of room temperature storage and at least 21 months of storage at refrigerated temperatures. This thermostable vaccine platform can significantly reduce distribution challenges for current and future vaccines, particularly in settings where it is challenging to maintain a cold chain.

Accordingly, provided herein are such formulations (also referred to herein as compositions), methods of making, and their method of use. The formulations are thermostable, lyophilized (NLC)-based formulations that form a cake when lyophilized with an appropriate cake-forming excipient and form an oil-in-water emulsion upon reconstitution. Techniques for generating NLC particles are known to those of ordinary skill in the art and described in J. H. Erasmus supra. Illustrative NLC particles have an oil core comprising a liquid phase lipid and a solid phase lipid surrounded by a cationic lipid, a hydrophobic surfactant, and a hydrophilic surfactant. In one implementation, the liquid phase lipid is squalene or synthetic squalene, the solid phase lipid is trimyristin, the cationic lipid is 1,2-dioleoyloxy-3-(trimethylammonio)propane (DOTAP), the hydrophobic surfactant is sorbitan monostearate, and the hydrophilic surfactant is polysorbate 80. The cake-forming excipient may be a saccharide such as a disaccharide for example sucrose and/or trehalose.

The NLC particles may be formulated in an appropriate aqueous medium, such as a sodium citrate solution, containing the cake-forming excipient. If a bioactive agent is added prior to lyophilization, a solution containing the bioactive agent may be combined with the NLC particles in the saccharide-containing solution. In an implementation, the aqueous solution with NLC particles may contain 20% w/v saccharide prior to lyophilization.

The NLC system itself displays long-term stability in liquid form at 4° C. maintaining its particle size and component concentrations, as well as retaining its ability to complex with and protect bioactive agents such as RNA. Due to this long-term stability, an NLC platform is suitable for stockpiling even before a specific pathogen is identified. A nucleotide encoding an appropriate antigen can be rapidly produced and complexed with pre-manufactured and stockpiled NLC particles. The NLC/bioactive agent complex may then be lyophilized with an appropriate cake-forming excipient and distributed without the need for cold-chain maintenance.

The compositions of this disclosure when lyophilized are thermostable for many months and are capable of the delivery of bioactive agents to cells. Delivery of the bioactive agent can be, for example, for the generation of an immune response and/or for treatment of disease and health conditions in a subject. The lyophilized compositions may be in the form of an elegant cake. The elegant cake may be a cake that does not exhibit browning, yellowing, shrinking, or cracking when stored at the conditioned indicated herein.

As provided herein, the lyophilized NLC composition is thermostable. For example, the NLC composition is thermostable at about 25° C. for at least 8 months and at about 4° C. for at least 21 months. Such compositions may further comprise suitable excipients, such as pharmaceutically acceptable excipients (carriers) including buffers, acids, bases, sugars, diluents, preservatives, and the like, which are well known in the art and are described herein. In yet another aspect, the invention provides methods for generating a thermostable, lyophilized vaccine composition described herein.

In some aspects, this disclosure provides methods for generating a thermostable, lyophilized vaccine platform or a thermostable, lyophilized vaccine when combined with a bioactive agent. The methods comprise generating NLC particles by mixing an oil phase mixture with an aqueous phase mixture. The oil phase mixture may comprise a liquid phase lipid, a cationic lipid, and a hydrophobic surfactant. The aqueous phase mixture may comprise a hydrophilic surfactant in an aqueous solution such as a sodium citrate solution. Optionally, a bioactive agent is added to the NLC particles. The NLC particles are then combined with a cake-forming excipient such as one or more saccharides and lyophilized. The cake-forming excipient may be present at a concentration of about 20% w/v prior to lyophilization. Lyophilization forms a cake that upon reconstitution forms an oil-in-water emulsion.

In some aspects, this disclosure provides methods for stimulating an immune response in a subject comprising reconstituting a thermostable, lyophilized vaccine composition described herein into an emulsion and administering the emulsion to the subject. In some implementations, the emulsion is an oil-in-water emulsion. In some implementations, the immune response is an antigen-specific immune response. A method described herein for stimulating an immune response, or a reconstituted thermostable lyophilized vaccine composition described herein, can be used alone or in combination with other conventional methods of treatment.

It is to be understood that one, some, or all of the properties of the various implementations described herein may be combined to form other implementations of the present invention. These and other aspects of the present invention will become evident upon reference to the following detailed description and attached drawings.

### BRIEF DESCRIPTION OF THE DRAWINGS

FIG. 1A is a schematic depicting RNA electrostatically binding to the outside of an illustrative NLC particle.

FIG. 1B shows the hydrodynamic diameter of NLC particle size over a 12-month period when stored as a liquid at the indicated temperatures.

FIG. 1C shows the stability of NLC component concentrations after long-term 4° C. storage in liquid form.

FIG. 1D shows stability in the hydrodynamic diameter of NLC particles complexed with SEAP saRNA after long-term 4° C. storage in liquid form.

FIG. 1E is an agarose gel stained with ethidium bromide that shows protection of SEAP saRNA from RNase challenge by NLC stored at 4° C. for the indicated length of time.

FIG. 2A shows lyophilized samples prior to reconstitution. Appearance of vials containing RNA complexed with NLC (top row), NLC alone (middle row) and RNA alone (bottom row).

FIG. 2B shows the lyophilized samples of FIG. 2A following reconstitution. Appearance of vials containing RNA complexed with NLC (top row), NLC alone (middle row) and RNA alone (bottom row).

FIG. 2C shows the effects of lyoprotectant on hydrodynamic diameter following freeze/thaw (F/T) and lyophilization of SEAP saRNA complexed with NLC. “Neat” indicates freshly prepared samples. Particle size growth was less when sucrose was used as a lyoprotectant relative to trehalose. Particle size growth increased 38% with 20% sucrose. With 10% sucrose there was greater particle growth.

FIG. 3A is an agarose gel stained with ethidium bromide that shows integrity of Zika saRNA under fresh or lyophilized/reconstituted conditions after extraction from the NLC and protection of Zika saRNA after challenge with RNase while lyophilized with the NLC (“Lyophilized—Challenged”). The fresh and lyophilized/reconstituted vaccine were also evaluated under un-challenged and challenged conditions after 2 weeks of storage at 4° C.

FIG. 3B shows in vivo immunogenicity equivalence of fresh and lyophilized/reconstituted Zika vaccine by PRNT in mice (n=10/group) after intramuscular (IM) injection. SEAP NLC/saRNA was used as an in vivo negative control. Neutralizing antibody titers were determined by 50% plaque reduction neutralization test (PRNT₅₀). Data displayed as box-and-whisker plots displaying median, first and third quartile (box), and maximum/minimum (whiskers).

FIG. 3C shows a comparison of hydrodynamic diameter of fresh and lyophilized/reconstituted NLC particles complexed with Zika saRNA with a background of 10% w/v sucrose.

FIG. 4A is an agarose gel stained with ethidium bromide that shows comparison of RNA integrity of fresh, lyophilized, and frozen NLC particles complexed with mRNA encoding ovalbumin (OVA) following RNase challenge.

FIG. 4B shows a comparison of the hydrodynamic diameter of fresh, frozen, and lyophilized complexes of OVA NLC/mRNA with a background of 20% w/v sucrose. Data is shown as mean+/−standard deviation (n=3).

FIG. 5A shows that lyophilization of SEAP NLC/saRNA in 20% w/v sucrose retained emulsion characteristics. Appearance of vials containing emulsion before lyophilization (left), as lyophilized cake (middle), and after reconstitution of lyophilized cake (right).

FIG. 5B shows hydrodynamic diameter of SEAP NLC/saRNA complexes over 21 months while stored under the indicated conditions in comparison to a freshly complexed control.

FIG. 5C is an agarose gel stained with ethidium bromide that shows RNA integrity and protection from RNase challenge of lyophilized, frozen, and liquid SEAP NLC/saRNA complexes stored at the indicated temperatures for the indicated length of time.

FIG. 5D shows normalized in vivo SEAP expression for lyophilized, frozen, or liquid stored samples in comparison with freshly complexed material after long-term storage. Error bars indicate standard deviation.

FIG. 5E shows a comparison of in vivo SEAP expression at 21 months for lyophilized vaccine, frozen vaccine stored, and freshly-prepared vaccine with 10% sucrose group shown as negative control. Data is shown as mean+/−standard deviation (n=10).

FIG. 6A is an agarose gel stained with ethidium bromide that shows RNA integrity and protection from RNase challenge of lyophilized, frozen, and freshly complexed SARS-Cov-2 RNA complexed with NLC stored at the indicated temperatures for one month.

FIG. 6B depicts SARS-CoV-2 spike protein-specific IgG antibody titers induced in mouse sera by injection of SARS-CoV-2 NLC/saRNA vaccine with and without lyophilization and storage at various conditions and temperatures.

FIGS. 7A-D depict DNA plasmids from the attenuated TC-83 strain of Venezuelan equine encephalitis virus (VEEV) under the control of a T7 RNA polymerase promoter. FIG. 7A depicts a replicon containing self-amplifying viral RNAs encoding premembrane (prM) and envelope (E) genes of ZIKV strain H/PF/2013. FIGS. 7B and 7C depict replicons containing RNA encoding secreted human embryonic alkaline phosphatase (SEAP). FIG. 7D depicts a replicon containing self-amplifying viral RNAs encoding the SARS-CoV-2 spike protein.

### DETAILED DESCRIPTION

NLC in liquid form and lyophilized NLC provide useful vaccine platforms for stockpiling and distribution of vaccines in both pandemic and non-pandemic situations. The NLC formulation of this disclosure is stable as a liquid at 4° C. for at least two years. This allows for advance preparation and storage of a vaccine platform that can be combined with a range of different bioactive agents. The efficacy of NLC vaccines complexed with RNA has been previously established. Vaccines of NLC and self-amplifying RNA (saRNA) have been shown to induce high levels of neutralizing antibodies and protect mice against viral challenge with the Zika virus. (J. H. Erasmus supra; and U.S. Pat. Pub. No. 2020/0230056 A1). However, the inventors are unaware of any previous work testing the effect of lyophilization on NLC formulations.

The inventors have discovered that the physical characteristics of this NLC-based vaccine formulation allow for lyophilization of the NLC vaccine formulation alone (i.e., without an antigen) and NLC-formulated vaccines. The lyophilized NLC formulations form lyophilized cakes that are thermostable at room temperature or refrigerated temperatures for several months. Furthermore, both the freshly-complexed liquid and the lyophilized/reconstituted vaccines are stable for at least two weeks at refrigerated temperatures allowing for storage prior to administration without freezing.

Techniques for lyophilization to stabilize vaccines and biologics are known to those of ordinary skill in the art (0. S. Kumru supra; D. Chen supra; P. Fonte et al., Facts and evidences on the lyophilization of polymeric nanoparticles for drug delivery. J Control Release 225, 75-86 (2016); K. L. Jones et al., Long-term storage of DNA-free RNA for use in vaccine studies. Biotechniques 43, 675-681 (2007); B. Petsch et al., Protective efficacy of in vitro synthesized, specific mRNA vaccines against influenza A virus infection. Nat Biotechnol 30, 1210-1216 (2012); M. Alberer et al., Safety and immunogenicity of a mRNA rabies vaccine in healthy adults: an open-label, non-randomised, prospective, first-in-human phase 1 clinical trial. Lancet 390, 1511-1520 (2017)). In lyophilized drug products, non-reducing sugars act as lyoprotectants through multiple proposed mechanisms such as replacing water in hydrogen bonding with the components of the system or enclosing the system within the rigid sugar matrix of the dried state where enzymatic or other degradation is limited (S. Franze et al., Lyophilization of Liposomal Formulations: Still Necessary, Still Challenging. Pharmaceutics 10, (2018)).

While lyophilization of liposome-based formulations has been attempted for decades (S. Franze supra), it is notoriously difficult due to the liposome's physical structure (i.e., a lipid bilayer surrounding a core aqueous phase) which is disrupted by the freezing and drying steps of lyophilization. Recent published attempts at LNP/RNA complex lyophilization have been semi-successful at best, showing significant loss of RNA activity despite the addition of lyoprotectants (R. L. Ball supra; P. Zhao supra). While optimization of LNP lyophilization may yet be attempted (C. Chen et al., An overview of liposome lyophilization and its future potential. J Control Release 142, 299-311 (2010)), the technical challenge of redesigning and clinically testing lyophilizable liposome-based RNA vaccine delivery formulations is significant and without guaranteed success.

The inventors have discovered, surprisingly, that high concentrations of saccharide in the formulation prior to lyophilization improves the quality and stability of the lyophilized cake formed from NLC. The saccharide may be a disaccharide such as sucrose or trehalose. The saccharide may be present in the liquid composition prior to lyophilization at amounts of about 10-20% w/v or at about 20% w/v.

The disclosure demonstrates that NLC/RNA vaccines are able to be stored in lyophilized, liquid, and frozen forms for extended periods of time. NLC/RNA vaccines can be successfully lyophilized for long-term storage with the addition of a lyoprotectant. The lyoprotectant functions as a cake-forming excipient that promotes the formation of a dense, white, lyophilized cake and also serves to protect the components of the system against the stresses encountered during freezing and drying. Sucrose was identified as one effective lyoprotectant. RNA integrity and protection against RNase challenge is maintained after lyophilization/reconstitution as shown by agarose gel electrophoresis. Additionally, in vivo data show that following lyophilization and long-term storage, the NLC/RNA vaccines retain the ability to deliver expressible RNA to a subject.

Without being bound by theory, it is believed that multiple mechanisms contribute to the improved thermostability of NLC-based delivery formulations relative to current LNP-based formulations. First, the robust physical stability of the NLC allows for minimal growth in particle size, retention of constituent components, and maintenance of complexing compatibility for at least one year under refrigerated storage. Furthermore, the NLC system provides excellent protection to the RNA against RNases, presumably due to the electrostatic interaction between RNA's negatively-charged phosphate backbone and the positively-charged amine group of the NLC's cationic lipid component. This interaction drives RNA/NLC complex formation and protects the RNA from cleavage by RNases during long-term storage and after administration.

The NLC system is ideal for situations of pandemic response. NLC manufacture is straightforward and scalable because it employs similar processes and equipment as oil-in-water emulsion technology already employed in licensed vaccines—properties essential to best support large-scale pandemic response. For pandemic preparedness, the long-term refrigerator-stable NLC alone could be stockpiled to enable rapid response. Furthermore, as RNA of different lengths or with multiple genetic variations can be rapidly synthesized and complexed on the outside of the NLC, head-to-head comparisons of different RNA species is feasible and such a vaccine may be rapidly adapted to evolving viral variants or emerging pathogens. Finally, once an RNA vaccine candidate has been chosen, the potential for a lyophilized, heat-stable RNA vaccine drug product would maximize the speed and ease of vaccine distribution.

This NLC-based delivery technology combined with lyophilization represents a significant advance for RNA vaccines with potentially paradigm-shifting implications on vaccine manufacture, storage, distribution, and overall cost due to its thermostable properties.

### I. Definitions

The following terms have the following meanings unless otherwise indicated. Any undefined terms have their art recognized meanings.

In the present description, the terms “about,” “around,” “approximately,” and similar referents mean±20% of the indicated range, value, or structure, unless otherwise indicated.

The use of the alternative (e.g., “or”) should be understood to mean either one, both, or any combination thereof of the alternatives.

As used herein, the terms “include,” “have” and “comprise” are used synonymously, which terms and variants thereof are intended to be construed as non-limiting.

As used herein and in the appended claims, the singular forms “a,” “an,” and “the” include plural reference unless the context clearly indicates otherwise.

The terms thermostable lyophilized vaccine composition, lyophilized vaccine composition, lyophilized thermostable cake, and lyophilized cake are used interchangeably herein. These terms generally refer to a lyophilized oil-in-water stable emulsion comprising a biodegradable oil or metabolizable oil, cake-forming excipients used to produce the cake, and optionally one or more bioactive agents.

The term “alkyl” means a straight chain or branched, noncyclic or cyclic, unsaturated or saturated aliphatic hydrocarbon containing the indicated number of carbon atoms. Unsaturated alkyls contain at least one double or triple bond between adjacent carbon atoms.

The terms “polypeptide,” “peptide,” and “protein” are used interchangeably herein to refer to polymers of amino acids of any length. The polymer may be linear or branched, it may comprise modified nucleotides or amino acids, and it may be interrupted by non-nucleotides or non-amino acids. The terms also encompass a nucleotide or amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polynucleotides or polypeptides containing one or more analogs of a nucleotide or an amino acid (including, for example, unnatural amino acids, etc.), as well as other modifications known in the art.

The term “isolated” means the molecule has been removed from its natural environment.

“Purified” means that the molecule has been increased in purity, such that it exists in a form that is more pure than it exists in its natural environment and/or when initially synthesized and/or amplified under laboratory conditions. Purity is a relative term and does not necessarily mean absolute purity.

A “polynucleotide” or “nucleic acid,” as used interchangeably herein, refer to polymers of nucleotides of any length, include DNA and RNA. The nucleotides can be, for example, deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

The term “RNA integrity” as used herein means the quantity of intact RNA remaining after an event or passage of time. For example, RNA integrity may be evaluated following freezing, lyophilization, or storage. RNA integrity may be evaluated by both the size and strength of bands shown in agarose gel electrophoresis.

An “individual” or a “subject” is any vertebrate. Vertebrates include, but are not limited to humans, primates, farm animals (such as cows, pigs, sheep, chickens), sport animals, pets (such as cats, dogs, birds, horses), and rodents.

A “replicon” as used herein includes any genetic element, for example, a plasmid, cosmid, bacmid, phage or virus that is capable of replication largely under its own control. A replicon may be either RNA or DNA and may be single or double stranded.

The term liquid phase lipid refers to a lipid that, prior to mixing with any other component, is liquid at ambient temperature.

The term solid phase lipid refers to a lipid that, prior to mixing with any other component, is solid at ambient temperature.

Ambient temperature is between 15° C. and 25° C.

Cake-forming excipient and lyoprotectant are used herein interchangeably. A cake-forming excipient refers to a substance added to a liquid stable oil-in-water emulsion formulation prior to lyophilization which yields a cake following lyophilization. Upon reconstitution of the lyophilized cake, a stable emulsion forms, that is suitable for delivery of a bioactive agent including vaccine antigens or polynucleotides encoding vaccine antigens. As used herein, cake-forming excipients are those substances which do not disrupt an emulsion upon reconstitution of the lyophilized cake.

Excipients as used herein refers to substances other than the pharmacologically active drugs, which are included in the manufacturing process, or fill-finish process for storage or shipment of the pharmacologically active drug including, without limitation, lyophilization, and are contained in a finished pharmaceutical process.

The practice of the present disclosure will employ, unless otherwise indicated, conventional techniques of molecular biology, recombinant DNA, biochemistry, and chemistry, which are within the skill of the art. Such techniques are explained fully in the literature. See, e.g., Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); B. Perbal, A Practical Guide to Molecular Cloning (1984); the treatise, Methods in Enzymology (Academic Press, Inc., N.Y.); and in Ausubel et al., Current Protocols in Molecular Biology, John Wiley and Sons, Baltimore, Maryland (1989).

### II. Nanostructured Lipid Carriers

The present disclosure provides, inter alia, NLCs for delivery of a bioactive agent to a cell. The NLC compositions are made up of NLC particles comprising (a) an oil core comprising a liquid phase lipid and a solid phase lipid, (b) a cationic lipid (c) a hydrophobic surfactant, preferably a sorbitan ester (e.g., sorbitan monoester, diester, or triester), and (d) a surfactant (preferably, a hydrophilic surfactant). The NLCs of the present invention typically comprise an unstructured or amorphous solid lipid matrix made up of a mixture of blended solid and liquid lipids dispersed in an aqueous phase. One or more of the surfactants can be present in the oil phase, the aqueous phase, or at the interface between the oil and aqueous phase. In certain aspects the sorbitan ester and the cationic lipid are present at the interface between the oil and aqueous phase.

NLCs are particularly effective at delivering protein-encoding nucleic acid such as RNA. By manipulating certain components of the NLC, the levels of expression of the encoded protein can be increased. Thus, NLCs are not only capable of effectively delivering RNA, they are also able to improve the immune response to the encoded proteins.

A. Solid-Phase and Liquid-Phase Lipids

NLCs are composed of a blend of solid and liquid lipids. The liquid and solid lipids to be used in the NLCs can be any lipid capable of forming an unstructured or amorphous solid lipid matrix and forming a stable composition. The weight ratio of solid to liquid can vary widely, for example from 0.1:99.9 to 99.9:0.1. In some illustrative implementations, the solid lipids are mixed with liquid lipids in a solid:liquid lipid weight ratio of from about 70:30 to about 99.9:0.1 or from about 1:10 to about 1:30. In some aspects, the solid lipids are mixed with liquid lipids in a solid:liquid lipid weight of about 1:16.

The total oil core component (solid lipid+liquid oil) of the NLC-based composition or formulation is typically present in an amount from about 0.2% to about 50% (w/v). For example, the NLC may comprise from about 0.2% to about 50% (w/v) oil core component, 0.2% to about 40% (w/v) oil core component, from about 0.2% to about 30% (w/v) oil core component, from about 0.2% to about 20% (w/v) oil core component, from about 0.2% to about 15% (w/v) oil core component, from about 0.2% to about 10% (w/v) oil core component, from about 0.2% to about 9% (w/v) oil core component, from about 0.2% to about 8% (w/v) oil core component, from about 0.2% to about 7% (w/v) oil core component, from about 0.2% to about 6% (w/v) oil core component, from about 0.2% to about 5% (w/v) oil core component, from about 0.2% to about 4.3% (w/v) oil core component, from about 0.3% to about 20% (w/v) oil core component, from about 0.4% to about 20% (w/v) oil core component, from about 0.5% to about 20% (w/v) oil core component, from about 1% to about 20% (w/v) oil core component, from about 2% to about 20% (w/v) oil core component, from about 3% to about 20% (w/v) oil core component, from about 4% to about 20% (w/v) oil core component, from about 5% to about 20% (w/v) oil core component, about 0.5% (w/v) oil core component, about 1% (w/v) oil core component, about 1.5% (w/v) oil core component, about 2% (w/v) oil core component, about 2.5% (w/v) oil core component, about 3% (w/v) oil core component, about 3.5% (w/v) oil core component, about 4% (w/v) oil core component, about 4.3% (w/v) oil core component, about 5% (w/v) oil core component, or about 10% (w/v) oil core component or any other amount or range described herein for the oil core component. Higher or lower w/v percentages are contemplated herein, particularly when considering diluted or concentrated formulations.

The oil core of the NLC comprises a liquid phase lipid. Preferably, although not necessarily, the liquid phase lipid is a metabolizable, non-toxic oil; more preferably one of about 6 to about 30 carbon atoms including, but not limited to, alkanes, alkenes, alkynes, and their corresponding acids and alcohols, the ethers and esters thereof, and mixtures thereof. The oil may be, for example, any vegetable oil, fish oil, animal oil or synthetically prepared oil that can be administered to a subject. In some aspects, the liquid phase lipid will be non-metabolizable.

The oil can be, for example, any long chain alkane, alkene or alkyne, or an acid or alcohol derivative thereof either as the free acid, its salt or an ester such as a mono-, or di- or triester, such as the triglycerides and esters of 1,2-propanediol or similar poly-hydroxy alcohols. Alcohols may be acylated employing a mono- or poly-functional acid, for example acetic acid, propanoic acid, citric acid or the like. Ethers derived from long chain alcohols which are oils and meet the other criteria set forth herein may also be used.

The individual alkane, alkene or alkyne moiety and its acid or alcohol derivatives will generally have from about 6 to about 40 or from 6 to about 30 carbon atoms. The moiety may have a straight or branched chain structure. It may be fully saturated or have one or more double or triple bonds. Where mono or poly ester- or ether-based oils are employed, the limitation of about 6 to about 40 carbons applies to the individual fatty acid or fatty alcohol moieties, not the total carbon count.

Any suitable oils from an animal, fish or vegetable source may be used. Sources for vegetable oils include nuts, seeds and grains, and suitable oils include, for example, peanut oil, soybean oil, coconut oil, and olive oil and the like. Other suitable seed oils include safflower oil, cottonseed oil, sunflower seed oil, sesame seed oil and the like. In the grain group, corn oil, and the oil of other cereal grains such as wheat, oats, rye, rice, teff, triticale and the like may also be used. The technology for obtaining vegetable oils is well developed and well known. The compositions of these and other similar oils may be found in, for example, the Merck Index, and source materials on foods, nutrition, and food technology.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Naturally occurring or synthetic terpenoids, also referred to as isoprenoids, can be used herein as a liquid phase lipid. Squalene, is a branched, unsaturated terpenoid. A major source of squalene is shark liver oil, although plant oils (primarily vegetable oils), including amaranth seed, rice bran, wheat germ, and olive oils, are also suitable sources. Squalane is the saturated analog to squalene. Oils, including fish oils such as squalene and squalane, are readily available from commercial sources or may be obtained by methods known in the art. Oils to be used herein may also be made using synthetic means, including genetic engineering (e.g., oils made from bioengineered yeast, including squalene.) Synthetic squalene has been successfully produced from bioengineered yeast and exhibits immunomodulating characteristics equal to squalene obtained from sharks. (Mizuki Tateno et al., Synthetic Biology-derived triterpenes as efficacious immunomodulating adjuvants, Sci Rep 10, 17090 (2020).) Squalene has also been synthesized by the controlled oligomerization of isoprene. (Kevin Adlington et al., Molecular Design of Squalene/Squalane Countertypes via the Controlled Oligomerization of Isoprene and Evaluation of Vaccine Adjuvant Applications, Biomacromolecules, 17(1) pages 165-172 (2016).)

Illustrative liquid phase lipids that can be used in the present invention include, for example, castor oil, coconut oil, corn oil, cottonseed oil, evening primrose oil, fish oil, grapeseed oil, jojoba oil, lard oil, linseed oil, olive oil, peanut oil, safflower oil, sesame oil, soybean oil, squalene, squalane, sunflower oil, wheatgerm oil, mineral oil, capric/caprylic triglyceride (e.g., Myglyol®810, Myglyol®812, Labrafac™), vitamin E (e.g., TOS, TPGS), lauroyl polyoxylglycerides (e.g., Gelucire®44/14), monoacylglycerols (e.g., Myverol 18-99 K), soy lecithin (e.g., Epikuron™200), farnesene, or a combination thereof.

The liquid phase lipid can include for example, squalene, sunflower oil, soybean oil, olive oil, grapeseed oil, squalane, capric/caprylic triglyceride, or a combination thereof.

The liquid phase lipid can include for example, squalene, squalene, capric/caprylic triglyceride, or a combination thereof.

The liquid phase lipid can include for example, capric/caprylic triglyceride, vitamin E, lauroyl polyoxylglycerides, monoacylglycerols, soy lecithin, squalene, or squalane or a combination thereof.

The liquid phase lipid can include for example, squalene, squalene, or farnesene or a combination thereof.

The oil core of the NLC comprises a solid phase lipid. A wide variety of solid phase lipids can be used, including for example, glycerolipids. Glycerolipids are fatty molecules composed of glycerol linked esterically to a fatty acid. Glycerolipids include triglycerides and diglycerides.

Illustrative solid phase lipids include, for example, glyceryl palmitostearate (Precitol ATO®5), glycerylmonostearate, glyceryl dibehenate (Compritol®888 ATO), cetyl palmitate (Crodamol™ CP), stearic acid, tripalmitin, or a microcrystalline triglyceride. Illustrative microcrystalline triglycerides include those sold under the trade name Dynasan® (e.g., trimyristin (Dynasan®114) or tristearin (Dynasan®118) or tripalmitin (Dynasan®116)).

The solid phase lipid can be, for example, a microcrystalline triglyceride, for example, one selected from trimyristin (Dynasan®114) or tristearin (Dynasan®118).

Preferably, the solid phase lipid of the oil core is solid at ambient temperature. When indoors, ambient temperature is typically between 15° C. and 25° C.

In any of the implementations provided herein, the solid phase lipid can be a glycerolipid, for example, a microcrystalline triglyceride.

In any of the implementations provided herein, the liquid phase lipid can be synthetic or naturally-occurring squalene.

B. Cationic Lipid

The NLCs described herein comprise a cationic lipid. The cationic lipid is useful for interacting with negatively charged bioactive agents on the surface on the NLC. Any cationic lipid capable of interacting with negatively charged bioactive agents that will not disturb the stability of the NLC and can be administered to a subject may be used. Generally, the cationic lipid contains a nitrogen atom that is positively charged under physiological conditions. Suitable cationic lipids include, benzalkonium chloride (BAK), benzethonium chloride, cetrimide (which contains tetradecyltrimethylammonium bromide and possibly small amounts of dodecyltrimethylammonium bromide and hexadecyltrimethyl ammonium bromide), cetylpyridinium chloride (CPC), cetyl trimethylammonium chloride (CTAC), primary amines, secondary amines, tertiary amines, including but not limited to N,N′,N′-polyoxyethylene (10)-N-tallow-1,3-diaminopropane, other quaternary amine salts, including but not limited to dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride, N,N-dimethyl-N-[2 (2-methyl-4-(1,1,3,3tetramethylbutyl)-phenoxy]-ethoxy)ethyl]-benzenemetha-naminium chloride (DEBDA), dialkyldimethylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3(dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12Bu6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C12GluPhCnN+), ditetradecyl glutamate ester with pendant amino group (C14GluCnN+), cationic derivatives of cholesterol, including but not limited to cholesteryl-3β-oxysuccinamidoethylenetrimethylammonium salt, cholesteryl-3β-oxysuccinamidoethylenedimethyl amine, cholesteryl-3β-carboxyamidoethylenetrimethyl ammonium salt, cholesteryl-3β-carboxyamidoethylenedimethyl amine, and 3γ-[N-(N′,N-dimethylaminoetanecarbomoyl]cholesterol) (DC-Cholesterol), 1,2-dioleoyloxy-3-(trimethylammonio)propane (DOTAP), dimethyldioctadecylammonium (DDA), 1,2-Dimyristoyl-3-TrimethylAmmoniumPropane (DMTAP), dipalmitoyl(C16:0)trimethyl ammonium propane (DPTAP), distearoyltrimethylammonium propane (DSTAP), and combination thereof.

Other cationic lipids suitable for use in the invention include, e.g., the cationic lipids described in U.S. Patent Pub. No. 2008/0085870 (published Apr. 10, 2008) and 2008/0057080 (published Mar. 6, 2008).

Other cationic lipids suitable for use in the invention include, e.g., Lipids E0001-E0118 or E0119-E0180 as disclosed in Table 6 (pages 112-139) of WO 2011/076807 (which also discloses methods of making, and method of using these cationic lipids). Additional suitable cationic lipids include N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), 1,2-dilinoleyloxy-3-dimethylaminopropane (DLinDMA).

The NLCs may comprise one or any combination of two or more of the cationic lipids described herein.

In illustrative implementations, the cationic lipid is selected from the group consisting of 1,2-dioleoyloxy-3-(trimethylammonio)propane (DOTAP), 313-[N-(N′,N′-Dimethylaminoethane)-carbamoyl]Cholesterol (DC Cholesterol), dimethyldioctadecylammonium (DDA), 1,2-Dimyristoyl-3-TrimethylAmmoniumPropane (DMTAP), dipalmitoyl(C16:0)trimethyl ammonium propane (DPTAP), distearoyltrimethylammonium propane (DSTAP), Lipids E0001-E0118 or E0119-E0180 as disclosed in Table 6 (pages 112-139) of WO 2011/076807, and combinations thereof.

In other illustrative implementations, the cationic lipid is selected from the group consisting of 1,2-dioleoyloxy-3-(trimethylammonio)propane (DOTAP), 313-[N-(N′,N′-Dimethylaminoethane)-carbamoyl]Cholesterol (DC Cholesterol), dimethyldioctadecylammonium (DDA), 1,2-Dimyristoyl-3-TrimethylAmmoniumPropane (DMTAP), dipalmitoyl(C16:0)trimethyl ammonium propane (DPTAP), distearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), 1,2-dilinoleyloxy-3-dimethylaminopropane (DLinDMA), Lipids E0001-E0118 or E0119-E0180 as disclosed in Table 6 (pages 112-139) of WO 2011/076807, and combinations thereof.

Illustrative cationic lipids are selected from the following: 1,2-dioleoyloxy-3-(trimethylammonio)propane (DOTAP), 3β-[N-(N′,N′-Dimethylaminoethane)-carbamoyl]Cholesterol (DC Cholesterol), dimethyldioctadecylammonium (DDA), 1,2-Dimyristoyl-3-TrimethylAmmoniumPropane (DMTAP), dipalmitoyl (C16:0)trimethyl ammonium propane (DPTAP), distearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), 1,2-dilinoleyloxy-3-dimethylaminopropane (DLinDMA), or a combination thereof. Additional suitable cationic lipids may be known by one of skill in the art.

In certain implementations, the NLC-based composition or formulation comprises from about 0.5 mg/ml to about 50 mg/ml of the cationic lipid. In certain implementations, the cationic lipid is DOTAP. The NLC may comprise, for example, from about 0.5 mg/ml to about 25 mg/ml or 30 mg/ml DOTAP or any other amount or range described herein for DOTAP.

In certain implementations, the cationic lipid is DC Cholesterol. In certain aspects, the NLC may comprise DC Cholesterol at from about 0.1 mg/ml to about 5 mg/ml DC Cholesterol. In certain implementations, the cationic lipid is DDA. The NLC may comprise, for example, from about 0.1 mg/ml to about 5 mg/ml DDA. In certain implementations, the cationic lipid is DOTMA. The NLC may comprise, for example, from about 0.5 mg/ml to about 25 or 30 mg/ml DOTMA. In certain implementations, the cationic lipid is DOEPC. The NLC may comprise, for example, from about 0.5 mg/ml to about 25 mg/ml DOEPC. In certain implementations, the cationic lipid is DSTAP. The NLC may comprise, for example, from about 0.5 mg/ml to about 50 mg/ml DSTAP. In certain implementations, the cationic lipid is DODAC. The NLC may comprise, for example, from about 0.5 mg/ml to about 50 mg/ml DODAC. In certain implementations, the cationic lipid is DODAP. The NLC may comprise, for example, from about 0.5 mg/ml to about 50 mg/ml DODAP.

With respect to weight per volume, an illustrative NLC-based composition or formulation may comprise, for example, from about 0.05% to about 5% or to about 10% w/v cationic lipid such as DOTAP, from about 0.2% to about 10% w/v cationic lipid such as DOTAP, from about 0.2% to about 5% w/v cationic lipid such as DOTAP, from about 0.2% to about 2% w/v cationic lipid such as DOTAP, from about 2% to 10% w/v cationic lipid such as DOTAP, from about 2% to about 5% w/v cationic lipid such as DOTAP, from about 1% to about 5% w/v cationic lipid such as DOTAP, from about 3% to about 5% w/v cationic lipid such as DOTAP, or from about 3% to about 4% w/v cationic lipid such as DOTAP or any other amount or range described herein for the cationic lipid. Higher or lower w/v percentages are contemplated herein, particularly when considering diluted or concentrated formulations.

In some cases, it may be desirable to use a cationic lipid that is soluble in the oil core. For example, DOTAP DOEPC, DODAC, and DOTMA are soluble in squalene or squalane. In other cases, it may be desirable to use a cationic lipid that is not soluble in the oil core. For example, DDA and DSTAP are not soluble in squalene. It is within the knowledge in the art to determine whether a particular lipid is soluble or insoluble in the oil and choose an appropriate oil and lipid combination accordingly. For example, solubility can be predicted based on the structures of the lipid and oil (e.g., the solubility of a lipid may be determined by the structure of its tail). For example, lipids having one or two unsaturated fatty acid chains (e.g., oleoyl tails), such as DOTAP, DOEPC, DODAC, DOTMA, are soluble in squalene or squalane; whereas lipids having saturated fatty acid chains (e.g., stearoyl tails) are not soluble in squalene. Alternatively, solubility can be determined according to the quantity of the lipid that dissolves in a given quantity of the oil to form a saturated solution).

The NLC may comprise additional lipids (i.e., neutral and anionic lipids) in combination with the cationic lipid so long as the net surface charge of the NLC prior to mixing with the bioactive agent is positive. Methods of measuring surface charge of a NLC are known in the art and include for example, as measured by Dynamic Light Scattering (DLS), Photon Correlation Spectroscopy (PCS), or gel electrophoresis.

C. Sorbitan Monoester

A sorbitan ester when added to the NLC can act to enhance the effectiveness of the NLC in delivering the bioactive agent to a cell and/or in eliciting antibodies to an antigen in a subject where the bioactive agent is an antigen or encodes antigen and the composition is administered to a subject. The term “sorbitan ester” as used herein refers to an ester of sorbitan. Sorbitan is as shown in Formula A

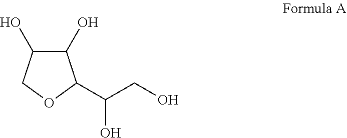 [SMILES: OCC(O)C1OCC(O)C1O]

Suitable sorbitan esters are sorbitan alkyl esters, wherein the alkyl is a C₁-C₃₀ alkyl group, preferably a saturated or unsaturated C₁-C₂₀ alkyl group, more preferably a saturated or unsaturated C₁₀-C₂₀ alkyl group.

In particular, it was discovered that the immune response to encoded proteins in the bioactive nucleic acid can be modulated by selection of sorbitan ester used in the NLC. It was surprisingly discovered that use of a sorbitan monoester was particularly effective at enhancing the effectiveness of the NLC. In some aspects, the acyl chain of the sorbitan monoester is saturated. In addition, without being bound by theory, it was surprisingly discovered that the sorbitan ester, and in particular, sorbitan monoester, acts in combination with the solid lipid (e.g., microcrystalline triglycerides) to enhance the effectiveness of the adjuvant activity of the NLC (e.g., in eliciting antibodies to an antigen in a subject where the bioactive agent is an antigen or encodes antigen and the composition is administered to a subject).

Illustrative sorbitan monoesters are commercially available under the tradenames SPAN® or ARLACEL®. An illustrative sorbitan monoester for use herein can be represented as a compound of Formula I or a stereoisomer thereof (including, but not limited to, Formula Ia, Ib, Ic, or Id) wherein R is a saturated or unsaturated C1-C30 alkyl group, preferably a saturated or unsaturated C1-C20 alkyl group, more preferably a saturated or unsaturated C10-C20 alkyl group. In illustrative implementations, the alkyl group is non-cyclic. Illustrative sorbitan monoesters also include positional isomers of Formulas I, Ia, Ib, Ic or Id (e.g., one of the hydroxy functional groups is replaced by an ester functional group (e.g., an alkyl ester wherein the alkyl is a saturated or unsaturated C1-C30 alkyl group, preferably a saturated or unsaturated C1-C20 alkyl group, more preferably a saturated or unsaturated C10-C20 alkyl group and R is OH). The skilled artisan will appreciate that illustrative sorbitan monoesters may be salt forms (e.g., pharmaceutically acceptable salts) of Formulas I, Ia, Ib, Ic, Id and stereoisomers or positional isomers thereof.

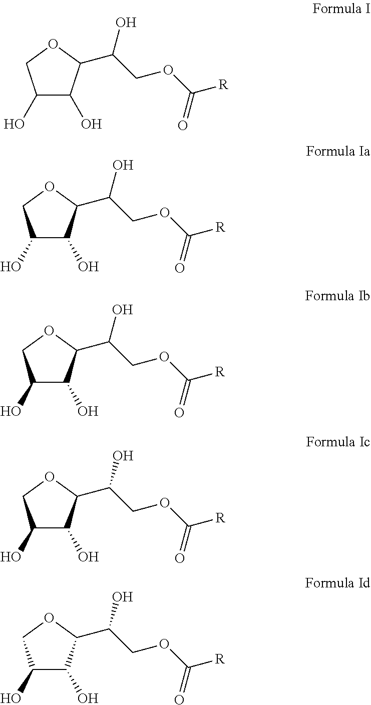

Suitable sorbitan monoesters in this regard are sorbitan monostearate (also knowns as Span®60 and shown below) and sorbitan monooleate (also known as Span®80 and shown below), although other sorbitan monoesters can be used (including, but not limited to, sorbitan monolaurate (Span®20), sorbitan monopalmitate (Span®40)). Illustrative sorbitan monostearate is represented by Formula II or IIa or a salt form thereof and illustrative sorbitan monooleate is represented by Formula III or IIIa or a salt form thereof.

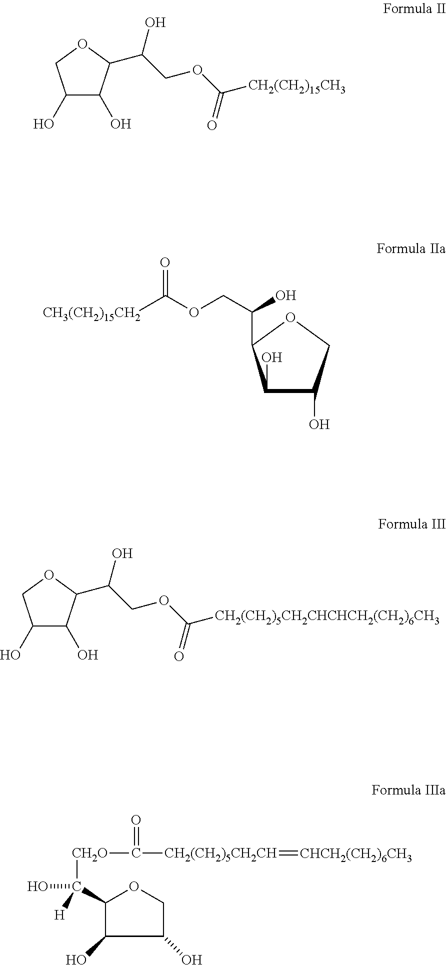

In addition to providing sorbitan monoesters as a component of a NLC, also contemplated is the substitution of the sorbitan monoester for an alternative hydrophobic surfactant, including alternative sorbitan-based non-ionic surfactants. Accordingly, also provided herein are NLC particles comprising an oil core comprising a liquid phase lipid and a solid phase lipid, a cationic lipid, a hydrophobic surfactant (e.g., non-ionic surfactants including sorbitan-based non-ionic surfactants) and a hydrophilic surfactant. Sorbitan-based non-ionic surfactants include sorbitan esters other than sorbitan monoesters, for example sorbitan diesters and sorbitan triesters, such as for example, sorbitan trioleate (SPAN85™) and sorbitan tristearate (SPAN65™). Generally, the non-ionic surfactant (including sorbitan-based non-ionic surfactant) will have a hydrophilic-lipophilic balance (HLB) number between 1.8 to 8.6. All of the implementations provided herein for the NLCs comprising a sorbitan monoester are applicable and contemplated for the NLCs comprising an alternative hydrophobic surfactant in place of the sorbitan monoester, e.g., NLCs comprising a sorbitan diester or triester in place of the sorbitan monoester. The sorbitan diester and triester or other hydrophobic surfactant can be present in the same concentrations as the sorbitan monoester. In some aspects, the acyl chains of the sorbitan diester or triester will be saturated.

Generally, the sorbitan esters (e.g., sorbitan monoesters) have a hydrophile-lipophile balance (HLB) value from 1 to 9. In some implementations, the sorbitan esters (e.g., sorbitan monoesters) have an HLB value from 1 to 5. In some implementations, the hydrophobic surfactant has a HLB value from about 4 to 5.

An illustrative sorbitan diester for use herein can be represented as a compound of Formula IV below or a stereoisomer thereof (e.g., wherein R is a saturated or unsaturated C1-C30 alkyl group, preferably a saturated or unsaturated C1-C20 alkyl group, more preferably a saturated or unsaturated C10-C20 alkyl group and at least one of R1 is H while the other is —C(═O)Y wherein Y is a saturated or unsaturated C1-C30 alkyl group, preferably a saturated or unsaturated C1-C20 alkyl group, more preferably a saturated or unsaturated C10-C20 alkyl group). In illustrative implementations, the alkyl group is non-cyclic. Illustrative sorbitan diesters also include positional isomers of Formulas IV. The skilled artisan will appreciate that illustrative sorbitan diesters may be salt forms (e.g., pharmaceutically acceptable salts) of Formula IV and stereoisomers or positional isomers thereof.

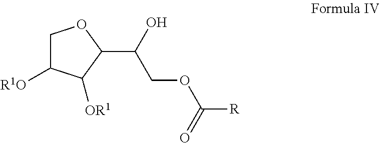

As illustrative sorbitan triester for use herein can be represented as a compound of Formula V below or a stereoisomer thereof (including, but not limited to, Formula Va, Vb, or Vc) wherein R is a saturated or unsaturated C1-C30 alkyl group, preferably a saturated or unsaturated C1-C20 alkyl group, more preferably a saturated or unsaturated C10-C20 alkyl group and R1 is —C(═O)Y wherein Y can be the same or different in each instance and is a saturated or unsaturated C1-C30 alkyl group, preferably a saturated or unsaturated C1-C20 alkyl group, more preferably a saturated or unsaturated C10-C20 alkyl group. In illustrative implementations, the alkyl group is non-cyclic. Illustrative sorbitan triesters also include positional isomers of Formulas V, Va, Vb, or Vc (e.g., the hydroxy functional group is replaced by an ester functional group (e.g., an alkyl ester wherein the alkyl is a saturated or unsaturated C1-C30 alkyl group, preferably a saturated or unsaturated C1-C20 alkyl group, more preferably a saturated or unsaturated C10-C20 alkyl group) and one of the alkyl esters (e.g., a ring alkyl ester or non-ring alkyl ester) is replaced by a hydroxy functional group). The skilled artisan will appreciate that illustrative sorbitan triesters may be salt forms (e.g., pharmaceutically acceptable salts) of Formulas V, Va, Vb, or Vc and stereoisomers or positional isomers thereof.

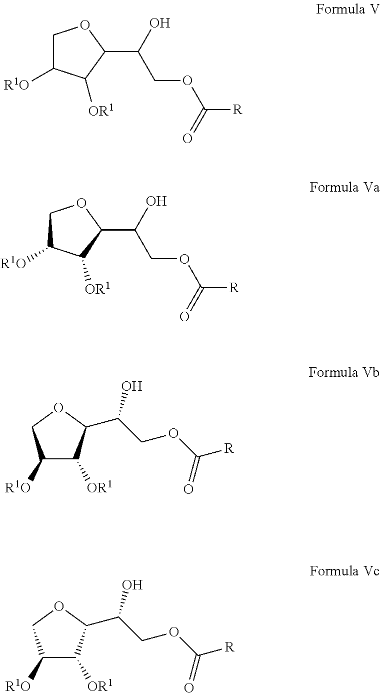

With respect to stereoisomers, the skilled artisan will understand that the sorbitan esters may have chiral centers and may occur, for example, as racemates, racemic mixtures, and as individual enantiomers and diastereomers.

In implementations wherein the sorbitan-based non-ionic surfactants is a sorbitan ester, typically, the NLC-based composition or formulation typically contains, for example, from about 0.1% to about 15% sorbitan ester (w/v), 0.1% to about 10% sorbitan ester (w/v), from 0.1% to about 5% sorbitan ester (w/v), about 0.1% to about 4% sorbitan ester (w/v), about 0.1% to about 4% sorbitan ester (w/v), about 0.1% to about 2.5% sorbitan ester (w/v), about 0.1% to about 2% sorbitan ester (w/v), 0.1% to about 1.5% sorbitan ester (w/v), 0.1% to about 1% sorbitan ester (w/v), 0.1% to about 0.5% sorbitan ester (w/v), 0.3% to about 2.5% sorbitan ester (w/v), about 0.3% to about 2% sorbitan ester (w/v), 0.3% to about 1.5% sorbitan ester (w/v), 0.3% to about 1% sorbitan ester (w/v), 0.3% to about 0.5% sorbitan ester (w/v) or any other amount or range described herein for a sorbitan ester, including from about 0.25% to about 15% sorbitan ester. In some aspects, the NLC-based compositions contain about 0.1%, about 0.2%, about 0.3%, about 0.4%, about 0.5%, about 0.6%, about 0.7%, about 0.8%, about 0.9%, about 1%, about 2%, about 3% or about 4% (w/v) sorbitan ester. Higher or lower w/v percentages are contemplated herein, particularly when considering diluted or concentrated formulations.

Accordingly, when the sorbitan ester is a sorbitan monoester (e.g., SPAN60™, SPAN80™), the NLC-based composition or formulation typically contains, for example, from about 0.1% to about 15% sorbitan monoester (w/v), 0.1% to about 10% sorbitan monoester (w/v), from 0.1% to about 5% sorbitan monoester (w/v), about 0.1% to about 4% sorbitan monoester (w/v), about 0.1% to about 4% sorbitan monoester (w/v), about 0.1% to about 2.5% sorbitan monoester (w/v), about 0.1% to about 2% sorbitan monoester (w/v), 0.1% to about 1.5% sorbitan monoester (w/v), 0.1% to about 1% sorbitan monoester (w/v), 0.1% to about 0.5% sorbitan monoester (w/v), 0.3% to about 2.5% sorbitan monoester (w/v), about 0.3% to about 2% sorbitan monoester (w/v), 0.3% to about 1.5% sorbitan monoester (w/v), 0.3% to about 1% sorbitan monoester (w/v), 0.3% to about 0.5% sorbitan monoester (w/v) or any other amount or range described herein for sorbitan monoester, including from about 0.25% to about 15% sorbitan monoester. In some aspects, the NLC-based composition or formulation contains about 0.1%, about 0.2%, about 0.3%, about 0.4%, about 0.5%, about 0.6%, about 0.7%, about 0.8%, about 0.9%, or about 1%, about 2%, about 3% or about 4% (w/v) sorbitan monoester. Higher or lower w/v percentages are contemplated herein, particularly when considering diluted or concentrated formulations.

Accordingly, when the sorbitan ester is a sorbitan diester, the NLC-based composition or formulation typically contain, for example, from about 0.1% to about 15% sorbitan diester (w/v), 0.1% to about 10% sorbitan diester (w/v), from 0.1% to about 5% sorbitan diester (w/v), about 0.1% to about 4% sorbitan diester (w/v), about 0.1% to about 4% sorbitan diester (w/v), about 0.1% to about 2.5% sorbitan diester (w/v), about 0.1% to about 2% sorbitan diester (w/v), 0.1% to about 1.5% sorbitan diester (w/v), 0.1% to about 1% sorbitan diester (w/v), 0.1% to about 0.5% sorbitan diester (w/v), 0.3% to about 2.5% sorbitan diester (w/v), about 0.3% to about 2% sorbitan diester (w/v), 0.3% to about 1.5% sorbitan diester (w/v), 0.3% to about 1% sorbitan diester (w/v), 0.3% to about 0.5% sorbitan diester (w/v) or any other amount or range described herein for sorbitan diester, including from about 0.25% to about 15% sorbitan diester. In some aspects, the NLC-based composition or formulation contains about 0.1%, about 0.2%, about 0.3%, about 0.4%, about 0.5%, about 0.6%, about 0.7%, about 0.8%, about 0.9%, or about 1%, about 2%, about 3% or about 4% (w/v) sorbitan diester. Higher or lower w/v percentages are contemplated herein, particularly when considering diluted or concentrated formulations.

Accordingly, when the sorbitan ester is a sorbitan triester (e.g., SPAN85™ or SPAN65™), the NLC-based composition or formulation typically contain, for example, from about 0.1% to about 15% sorbitan triester (w/v), 0.1% to about 10% sorbitan triester (w/v), from 0.1% to about 5% sorbitan triester (w/v), about 0.1% to about 4% sorbitan triester (w/v), about 0.1% to about 4% sorbitan triester (w/v), about 0.1% to about 2.5% sorbitan triester (w/v), about 0.1% to about 2% sorbitan triester (w/v), 0.1% to about 1.5% sorbitan triester (w/v), 0.1% to about 1% sorbitan triester (w/v), 0.1% to about 0.5% sorbitan triester (w/v), 0.3% to about 2.5% sorbitan triester (w/v), about 0.3% to about 2% sorbitan triester (w/v), 0.3% to about 1.5% sorbitan triester (w/v), 0.3% to about 1% sorbitan triester (w/v), 0.3% to about 0.5% sorbitan triester (w/v) or any other amount or range described herein for sorbitan triester, including from about 0.25% to about 15% sorbitan triester. In some aspects, the NLC-based composition or formulation contains about 0.1%, about 0.2%, about 0.3%, about 0.4%, about 0.5%, about 0.6%, about 0.7%, about 0.8%, about 0.9%, about 1%, about 2%, about 3% or about 4% (w/v) sorbitan triester. Higher or lower w/v percentages are contemplated herein, particularly when considering diluted or concentrated formulations.

In illustrative implementations, the sorbitan ester (e.g., sorbitan monoester, diester or triester) is present in an amount sufficient to increase the ability of the composition to facilitate delivery and/or expression of the bioactive agent (e.g., RNA) as compared to a comparable composition lacking the sorbitan ester (e.g., sorbitan monoester, diester or triester respectively). In implementations where the composition is administered to the subject in an effective amount, the composition may elicit antibody titers to the antigen equal to or greater than the antibody titers elicited when a comparable composition lacking the sorbitan ester is administered to the subject or when the bioactive agent is administered to the subject without the NLC. In some implementations, the composition induces an immune response (e.g., neutralizing antibody titers) in the subject at a higher level than the immune response induced in the subject by a comparable composition lacking the sorbitan ester. Immune response may be, for example, innate, cellular or antibody responses. Neutralizing antibody titers may be determined by any assay known to one of skill in the art, including, without limitation, a plaque reduction neutralization titer analysis (Ratnam, S et al. J. Clin. Microbiol (2011), 33 (4): 811-815; Timiryazova, T et al. Am J Trop Med Hyg (2013), 88(5): 962-970).

D. Surfactants

The NLCs described herein comprise a surfactant, in addition to the sorbitan-based non-ionic surfactants (e.g., sorbitan ester). There are a number of surfactants specifically designed for and commonly used in biological applications. Such surfactants are divided into four basic types and can be used in the present invention: anionic, cationic, zwitterionic and nonionic. A particularly useful group of surfactants are the hydrophilic non-ionic surfactants and, in particular, polyoxyethylene sorbitan monoesters and polyoxyethylene sorbitan triesters. These materials are referred to as polysorbates and are commercially available under the mark TWEEN® and are useful for preparing the NLCs. TWEEN® surfactants generally have a HLB value falling between 9.6 to 16.7. TWEEN® surfactants are commercially available. Other non-ionic surfactants which can be used are, for example, polyoxyethylene fatty acid ethers derived from lauryl, acetyl, stearyl and oleyl alcohols, polyoxyethylene fatty acids made by the reaction of ethylene oxide with a long-chain fatty acid, polyoxyethylene, polyol fatty acid esters, polyoxyethylene ether, polyoxypropylene fatty ethers, bee's wax derivatives containing polyoxyethylene, polyoxyethylene lanolin derivative, polyoxyethylene fatty glycerides, glycerol fatty acid esters or other polyoxyethylene fatty acid, alcohol or ether derivatives of long-chain fatty acids of 12-22 carbon atoms.

In some implementations, it is preferable to choose a non-ionic surfactant which has an HLB value in the range of about 7 to 16. This value may be obtained through the use of a single non-ionic surfactant such as a TWEEN® surfactant or may be achieved by the use of a blend of surfactants. In certain implementations, the NLC comprises a single non-ionic surfactant, most particularly a TWEEN® surfactant, as the emulsion stabilizing non-ionic surfactant. In an illustrative implementation, the emulsion comprises TWEEN® 80, otherwise known as polysorbate 80.

The NLC-based composition or formulation contains can contain, for example, from about 0.01% to about 15% surfactant (w/v), from about 0.01% to about 10% surfactant (w/v) from about 0.01% to about 5% surfactant (w/v), about 0.01% to about 2.5% surfactant, about 0.01% to about 2% surfactant, 0.01% to about 1.5% surfactant, 0.01% to about 1% surfactant, 0.01% to about 0.5% surfactant, 0.05% to about 0.5% surfactant, 0.08% to about 0.5% surfactant, about 0.08% surfactant, about 0.5% surfactant, about 0.6% surfactant, about 0.7% surfactant, about 0.8% surfactant, about 0.9% surfactant, or about 1% surfactant, or about 2%, about 3%, about 4% surfactant or any other amount or range described herein for surfactant. Higher or lower w/v percentages are contemplated herein, particularly when considering diluted or concentrated formulations.

Additional components can be included in the NLCs of the present invention including, for examples, components that promote NLC formation, improve the complex formation between the negatively charged molecules and the cationic particles, facilitate appropriate release of the negatively charged molecules (such as an RNA molecule), and/or increase the stability of the negatively charged molecule (e.g., to prevent degradation of an RNA molecule).

The aqueous phase (continuous phase) of the NLCs is typically a salt solution (e.g., saline) or water. The salt solution is typically an aqueous solution that comprises a salt (e.g., sodium citrate), and can further comprise, for example, a buffer (e.g., a citrate buffer), an osmolality adjusting agent (e.g., a saccharide), a polymer, a surfactant, or a combination thereof. If the emulsions are formulated for parenteral administration, it is preferable to make up final solutions so that the tonicity, i.e., osmolality is essentially the same as normal physiological fluids in order to prevent undesired post-administration consequences, such as post-administration swelling or rapid absorption of the composition. It is also preferable to maintain a pH compatible with normal physiological conditions. Also, in certain instances, it may be desirable to maintain the pH at a particular level in order to ensure the stability of certain components of the NLC. For example, it may be desirable to prepare a NLC that is isotonic (i.e., the same permeable solute (e.g., salt) concentration as the normal cells of the body and the blood) and isosmotic. To control tonicity, the NLC may comprise a physiological salt, such as a sodium salt. In some aspects, sodium chloride (NaCl), for example, may be used at about 0.9% (w/v) (physiological saline). Other salts that may be present include, for example, potassium chloride, potassium dihydrogen phosphate, disodium phosphate, magnesium chloride, calcium chloride, and the like. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the present invention. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents that can be useful in the present invention. Non-ionic tonicity modifying agents can be present, for example, at a concentration of from about 0.1% to about 10% or about 1% to about 10%, depending upon the agent that is used.

The aqueous phase may be, but is not necessarily, buffered. Any physiologically acceptable buffer that provides adequate protection for the RNA may be used herein, such as water, citrate buffers, phosphate buffers, acetate buffers, tris buffers, bicarbonate buffers, carbonate buffers, succinate buffer, or the like. The pH of the aqueous component will preferably be between 4.0-8.0 or from about 4.5 to about 6.8. In another illustrative implementation, the aqueous phase is, or the buffer prepared using, RNase-free water or DEPC treated water. In some cases, high salt in the buffer might interfere with complexation of negatively charged molecule to the emulsion particle therefore is avoided. In other cases, certain amount of salt in the buffer may be included.

In an illustrative implementation, the aqueous solution is sodium citrate with a pH between about 5.0 and 8.0. The sodium citrate solution may have a concentration of between 1-20 mM such as, 5 mM, 10 mM, 15 mM, or 20 mM. In another illustrative implementation, the aqueous phase is, or the buffer is prepared using, RNase-free water or DEPC treated water.

The aqueous phase may also comprise additional components such as molecules that change the osmolarity of the aqueous phase or molecules that stabilize the negatively charged molecule after complexation. Preferably, the osmolarity of the aqueous phase is adjusting using a non-ionic tonicifying agent, such as a sugar (e.g., trehalose, sucrose, dextrose, fructose, reduced palatinose, etc.), a sugar alcohol (such as mannitol, sorbitol, xylitol, erythritol, lactitol, maltitol, glycerol, etc.), or combinations thereof. If desired, a nonionic polymer (e.g., a poly(alkyl glycol) such as polyethylene glycol, polypropylene glycol, or polybutlyene glycol) or nonionic surfactant can be used.

E. Excipients

Excipients may be used singly or in combination with other excipients which include, but are not limited to, cake-forming excipients, cake-forming bulking agents, bulking agents, buffering agents, chelating agents, solubilizing agents, isotonicity agents, tonicifying agents, surfactants, emulsifiers, antimicrobial agents, and/or collapse temperature modifiers.

The excipients are substances other than a bioactive agent, which are included in the manufacturing process, or fill-finish process for storage or shipment of the composition including, without limitation, lyophilization, and are contained in a finished vaccine platform or vaccine. An excipient is a substance added to a liquid stable oil-in-water emulsion formulation prior to lyophilization which yields a cake following lyophilization.

Excipients suitable for vaccine formulations and/or lyophilization are known in the art (See, e.g., Bahetia et. al., 2010: J. Excipients and Food Chem.: 1 (1)41-54, Grabenstein J D. ImmunoFacts: Vaccines and Immunologic Drugs—2012 (37th revision). St Louis, MO: Wo Iters Kluwer Health, 2011 and, by Vaccine) and include cake-forming excipients, cake-forming bulking agents, chelating agents, bulking agents, buffering agents, solubilizing agents, isotonicity agents, tonicifying agents, surfactants, emulsifiers, antimicrobial agents, and/or collapse temperature modifiers. Excipients in approved vaccines include without limitation sucrose, D-mannose, D-fructose, dextrose, potassium phosphate, plasdone C, anhydrous lactose, micro crystalline cellulose, polacrilin potassium, magnesium stearate, cellulose acetate phthalate, alcohol, acetone, castor oil, FD&C Yellow #6 aluminum lake dye, human serum albumin, fetal bovine serum, sodium bicarbonate, human-diploid fibroblast cell cultures (WI-38), Dulbecco's Modified Eagle's Medium, aluminum hydroxide, benzethonium chloride, formaldehyde, gluteraldehyde, amino acids, vitamins, inorganic salts, sugars, glycerin, asparagine, citric acid, potassium phosphate, magnesium sulfate, iron ammonium citrate, lactose, aluminum potassium sulfate, aluminum hydroxyphosphate, potassium aluminum sulfate, peptone, bovine extract, thimerosal (trace), modified Mueller and Miller medium, beta-propiolactone, thimerosol (multi-dose vials only), monobasic sodium phosphate, dibasic sodium phosphate, monobasic potassium phosphate, potassium chloride, potassium glutamate, calcium chloride, sodium taurodeoxycholate, neomycin sulfate, polymyxin B, egg protein, lactalbumin hydrolysate, and neomycin sulfate.

Chelating agents such as ethylenediaminetetraacetic acid (EDTA) may be present at concentrations of between about 0.1-1 mM.

Cake-Forming Excipients/Cake-Forming Bulking Agents

A cake-forming excipient is a substance added to a liquid stable oil-in-water emulsion formulation prior to lyophilization which yields a cake following lyophilization. Upon reconstitution of the lyophilized cake, an oil-in-water stable emulsion forms which is suitable for delivery of a pharmacologically active drug including the vaccines of the present invention. In some implementations, cake-forming excipients are those substances which do not disrupt an emulsion upon reconstitution of the cake.

In some implementations the agents useful as cake-forming excipients, also referred to as bulking agents, for the present invention include sugars/saccharides or sugars/saccharides in combination with sugar alcohols. In some implementations disclosed herein, the sugars/saccharides or sugars/saccharides in combination with sugar alcohols are useful as bulking agents or cake-forming excipients include. These include, but are not limited to, trehalose, dextrose, lactose, maltose, sucrose, raffinose, mannose, stachyose, fructose, lactulose, glucose, glycerol, sorbitol, and/or mannitol. In one implementation, the cake-forming excipient is sucrose. In one implementation, the cake-forming excipient is trehalose.

In some implementations, the cake-forming excipient is a saccharide and the saccharide is present in the NLC formulation prior to lyophilization at a concentration range of about 5% w/v to about 22% w/v, about 5% to about 20%, about 5% w/v to about 18% w/v, about 8% w/v to about 15% w/v, or about 9% w/v to about 11% w/v. In some implementations, the saccharide is present in the NLC formulation prior to lyophilization a concentration of about 5%, 6%, 7%, 8%, 9%, 10%, 11%, 12%, 13%, 14%, 15%, 16%, 17%, 18%, 19%, or 20%.

F. Buffering Agents

In some implementations, the compositions of the present invention comprise a buffering agent. Buffering agents useful as excipients in the present invention include Tris acetate, Tris base, Tris-HCl, ammonium phosphate, citric acid, sodium citrate, potassium citrate, tartic acid, sodium phosphate, zinc chloride, arginine, and histidine. Concentration of the buffering agents may range between 1-20 mM such as, for example 5 mM, 10 mM, or 20 mM. In some implementations buffering agents include pH adjusting agents such as hydrochloric acid, sodium hydroxide, and meglumine.

G. Oil: Surfactant Ratios

Illustrative NLCs are composed of a hydrophobic core containing the liquid oil and solid lipid, and surfactants (also known as emulsifiers or emulsifying agents) that make up the interface separating the hydrophobic phase—liquid oil and solid lipid, collectively referred to here as oil—from the aqueous phase. Since surfactants typically reside on the surface of NLC nanoparticles, their amount dictates the total available surface area. On the other hand, the oil resides in the core and primarily contributes to the total available volume. Increasing the surfactant to oil ratio consequently increases the surface area (SA) to volume ratio (V); thus, for a fixed volume of material, increasing the SA/V ratio translates to reducing NLC particle diameter. Instead of, or, in addition, to describing illustrative NLC compositions in terms of the w/v percentages of various components, they can be described by the molar ratios of various components. In some aspects, illustrative NLCs of the present invention, have an oil to surfactant molar ratio of from about 0.05 to about 12 or from about 0.05 to about 9 or from about 0.05 to about 8 or from about 0.05 to about 1 or from about 0.1 to about 1. By reducing the oil to surfactant molar ratio, smaller NLCs can be synthesized. In addition, by reducing the amount of oil in the NLCs, potential toxicity of the formulations can be reduced. In other aspects, illustrative NLCs of the present invention, have an oil to surfactant molar ratio of from about 0.5 to about 12, from about 0.5 to about 9, from 1 to about 9, from about 2 to about 9, from about 3 to about 9, from about 4 to about 9, from about 4.5 to about 9, or from about 4.5 or about 5 to about 7. Illustrative formulations have an oil to surfactant molar ratio of about 0.5, about 1, about 1.5, about 2, about 2.5, about 3, about 3.5, about 4, about 4.5, about 5, about 5.5, about 6, about 7, about 8, about 9, about 10, about 11, or about 12. As used herein, the oil to surfactant molar ratio is determined by (i) adding the moles of lipid that make up the oil core (solid phase lipid and liquid phase lipid) to arrive at a value for moles of oil core lipid (ii) adding the moles of the cationic lipid (e.g., DOTAP), hydrophobic surfactant (e.g., sorbitan ester) and hydrophilic surfactant (tween 80) to arrive at a value for moles surfactant, and (iii) dividing moles of oil core lipid by moles of surfactant.

H. Hydrophilic Surfactant: Cationic Lipid Ratios

The ratio of hydrophilic surfactant to cationic lipid can impact the ability of the NLC to have a protective effect from RNase degradation and can impact the immunogenicity of the formulations. In particular, Tween:DOTAP ratios at about 0.6 are beneficial for obtaining consistent results for delivery and expression of RNA bioactive agents whereas Tween:DOTAP ratios at about 2.0 and higher are not as beneficial for obtaining such consistency. Accordingly, illustrative NLCs of the present invention have a hydrophilic Surfactant:Cationic lipid (e.g., cationic lipid) ratio of from about 0.2 to about 1.5, from about 0.2 to about 1 or from about 0.5 to about 1. When Tween and DOTAP are in the composition, illustrative NLCs of the present invention have a tween:DOTAP ratio of from about 0.2 to about 1.5, from about 0.2 to about 1 or from about 0.5 to about 1. As used herein, the hydrophilic surfactant:cationic lipid ratio is determined by (i) adding the moles of hydrophilic surfactant to arrive at a value for moles of hydrophilic surfactant (ii) adding the moles of the cationic lipid to arrive at a value for moles of cationic lipid, and (iii) dividing moles of hydrophilic surfactant by moles of cationic lipid.

I. Loading Capacities

The loading capacity of the NLC formulations can be manipulated by modulating the ratio of hydrophilic surfactant to cationic lipid and the amount of oil present in the formulations thereby reducing the average NLC particle size. Illustrative NLC formulations have loading capacity for RNA of at least about 10 μg/ml RNA, at least about 20 μg/ml RNA, at least about 50 μg/ml RNA, at least about 100 μg/ml RNA, at least about 200 μg/ml RNA, at least about 300 μg/ml, or at least about 400 μg/ml RNA. NLC formulations having an average particle size of from 20 nm to about 110 nm, from about 20 nm to about 80 nm, from about 20 nm to about 70 nm, from about 20 nm to about 60 nm typically have increased loading capacity. Persons of ordinary skill in the art will appreciate how to adjust the NLC formulation to achieve a desired loading capacity.

### III. Physiochemical Characteristics of the Nanostructured Lipid Carriers

A. Size

The size of the NLC can be assessed by known techniques in the art, including but not limited to, x-ray and laser diffraction, dynamic light scattering (DLS), or CryoEM. In some implementations, the size of the NLC refers to the Z-average diameter.

The NLCs have an average diameter (i.e., the number average diameter) of 1 micrometer or less. It is particularly desirable that the average particle size (i.e., the number average diameter) of the NLC is about 900 nm or less, about 800 nm or less, about 700 nm or less, about 600 nm or less, about 500 nm or less, about 400 nm or less, 300 nm or less, 200 nm or less, 100 nm or less or 80 nm or less, for example, from about 50 nm to about 900 nm, from about 50 nm to about 800 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, from about 50 nm to about 175 nm, from about 50 nm to about 150 nm, from about 50 nm to about 125 nm, from about 50 nm to about 100 nm, from about 50 nm to about 80 nm, from about 40 nm to about 80 nm, from about 20 nm to about 80 nm, from about 40 nm to about 80 nm, or from about 40 nm to about 60 nm. It will be understood by the skilled practitioner that a NLC is made up of NLC particles. The average particle size refers to the average diameter of the particles that make up the NLC. The average diameter of the NLC particles is typically about 40 nm, is about 60 nm, is about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, or is about 200 nm.

In some aspects, the average diameter of the NLC particles is from about 20 nm to about 200 nm, from about 20 nm to about 150 nm, from about 20 nm to about 110 nm, from about 20 nm to about 80 nm, from about 20 nm to about 70 nm, from about 20 nm to about 60 nm.

In some aspects, the average diameter of the NLC particles is from about 50 nm to about 200 nm, from about 50 nm to about 150 nm, from about 50 nm to about 110 nm, from about 50 nm to about 80 nm, from about 50 nm to about 70 nm, from about 50 nm to about 60 nm.

In some aspects, the average diameter of the NLC particles is from about 40 nm to about 80 or from about 40 nm to about 60 nm.

An illustrative NLC of the present invention is capable of being filtered through at least a 0.45 micron filter. In an illustrative implementation, the NLC is capable of being filtered through a 0.20 or 0.22 micron filter.

B. Stability

Illustrative NLCs provided herein are stable, allowing for ease of use, manufacturability, transportability, and storage. The physiochemical characteristics of the NLC, including, but not limited to its size, is maintained over time, at various temperatures, and under various conditions.

The evolution of particle size over a function of time provides colloidal stability information. An illustrative stable NLC composition is one whose particles retain substantially the same z-average diameter size over a time period (e.g., a 30 day or 7 day time period) at different temperatures typically but not limited to 37, 25 or 5 degrees Celsius. By retaining substantially the same z-average diameter size, it is meant that a particle remains within 20%, 15%, 10%, 5%, of its original size over a 30 day time period. A particularly stable NLC composition is one whose particles retain substantially the same z-average diameter size over a six month period, an eight month period, a 12 month period, or a 21 month period at 4° C. or 25° C.

The stability of the NLC can be measured by techniques familiar to those of skill in the art. In some implementations, the stability is observed visually. Visual inspection can include inspection for particulates, flocculence, or aggregates. Typically, colloidal stability is determined by the particle size of the NLC, such as by measuring the z-average diameter and optionally expressed as change in size over time, or at various temperatures, or under certain conditions. In some implementations, the stability is determined by assessing the increase in particle size. In some implementations, stability is determined by measurement of the polydispersity index (PDI), for example with the use of the dynamic light scattering (DLS) technique. In other implementations, stability is determined by measurement of the zeta potential with the use of the DLS technique.

In some implementations, the Z-average diameter of the NLC increases less than 50%, less than 40%, less than 30%, less than 25%, less than 20%, less than 15%, less than 12%, less than 10%, less than 7%, less than 5%, less than 3%, less than 1% over the time period assayed.

In some implementations the polydispersity index of the NLC is maintained at about 0.5, at about 0.4, at about 0.3, at about 0.2, at about 0.1 or at from about 0.1 to about 0.5, at from about 0.1 to about 0.4, at from about 0.1 to about 0.3, at from about 0.1 to about 0.2, at from about 0.2 to about 0.4, or at from about 0.2 to about 0.3. In some aspects, the polydispersity index is greater than 0.1, greater than 0.15, or greater than 0.2.

Illustrative NLC-based compositions of the present invention when lyophilized are stable for at least 21 months at 4° C. and at least 8 months at 25° C. (e.g., retain substantially the same z-average diameter size).

### IV. Bioactive Agents

In some illustrative implementations, in order to deliver a bioactive agent, the formulations of the present invention are mixed or otherwise formulated with one or more bioactive agents. The term “bioactive agent” as used herein refers to any material to be delivered by the formulations of the present disclosure and can include without limitation macromolecules, peptides, proteins, peptidomimetics, nucleic acids, oligonucleotides, deoxyribonucleotides, plasmid DNA, circular DNA, linear DNA, single-stranded DNA, modified DNA, antisense DNA, ribonucleotides, mRNA, chemically modified RNA, non-coding RNA, miRNA, siRNA, tRNA, ribosomal RNA, RNA ribozymes, replicon RNA, self-amplifying RNA (saRNA), RNA aptamers, DNA aptamers, double-stranded RNA, base-substituted RNA, inosine-containing RNA, adjuvants including TLR agonists (for example TLR2, TLR3, TLR4, TLR 7, TLR8, and TLR9 agonists), Rig-I agonists, saponins, carbohydrates, carbohydrate polymers, conjugated carbohydrates, whole viral particles, virus-like particles, viral fragments, and cellular fragments. Nonlimiting illustrative adjuvants include double-stranded RNA, RIBOXXOL, poly(I:C), and Hiltonol® (poly-ICLC). Hiltonol® (poly-ICLC) is a synthetic complex of carboxymethylcellulose, polyinosinic-polycytidylic acid double-stranded RNA, and poly-L-lysine. RIBOXXOL is an annealed 50 bp RNA duplex (Riboxx GmbH). Any bioactive agent that can be delivered safely to a cell can be mixed with a NLC of the present invention. When negatively charged molecules are to be delivered, in some implementations, the cationic NLC surface can interact electrostatically with negatively charged bioactive agents thereby anchoring the molecules to the NLC.

Illustrative negatively charged molecules to be used as bioactve agents include, for example, peptide-containing antigens, nucleic acid molecules (e.g., RNA or DNA) that encode one or more peptide-containing antigens, negatively charged polysaccharides, negatively charged small molecules, and negatively charged immunological adjuvants. Negatively charged immunological adjuvants include, for example, immunostimulatory oligonucleotides (e.g., CpG oligonucleotides), single-stranded RNAs, small molecule immune potentiators (SMIPs), and the like. Negatively charged small molecules include, for example, phosphonate, fluorophosphonate, and the like.

Current adjuvants are largely Th2 biased, such as alum. In some implementations, for vaccines against cancer and infectious disease targets (e.g., tuberculosis, several viral diseases, etc.) as well as allergy, adjuvants that promote a Th1 bias are an unmet need. In this regard, formulations promoting a Th1 bias may be used. Such formulations promote IFN gamma production and downregulate IL-5 and are suitable for various uses in which a Th1 bias is desired.

One or more bioactive agents may be associated with the formulations of the present invention. One of skill in the art would understand that various combinations of bioactive agents may be associated with the formulations such as, but not limited to, multiple RNAs, multiple DNAs, one or more RNAs of a defined sequence and one or more proteins, one or more DNAs and one or more proteins, and one or more RNAs and one or more DNAs. In some aspects, one bioactive agent can be present in the oil core of an NLC while the other is associated with its surface of the NLC. For example, a nucleic acid may be associated with the NLC surface whereas a biologically active small molecule may be present within the oil core of the NLC.

In an illustrative implementation, the negatively charged bioactive agent is complexed with an NLC by association with the NLC's cationic surface. The association of the negatively charged bioactive agent with the NLC surface may be a non-covalent or a reversible covalent interaction. The association of the negatively charged bioactive agent with the NLC surface may be through electrostatic attraction.

In another implementation, a hydrophobic bioactive agent such as a Toll-like receptor ligand (e.g., TLR4 ligand) can be incorporated in the oily core or at the interface of the NLC particle.

A. RNA Molecules

In implementations where the bioactive agent is an RNA molecule, the RNA molecule may encode proteins of various types, including, without limitation, antigens, antibodies, toxins, growth factors, cytokines, and hormones. RNA molecules used herein may also represent non-coding RNAs, including, without limitation, mRNA, saRNA, siRNA, miRNA, CRISPR guide RNA, ribozyme RNA, hairpins, RNA aptamers, RNA agonists, and immunomodulatory RNAs.

In an illustrative implementation, the negatively charged RNA molecule is complexed with the NLC by association with the cationic surface. The association of the RNA molecule with the NLC surface may be a non-covalent or reversible covalent interaction. The non-covalent association may be electrostatic attraction.

In illustrative implementations, the bioactive agent is a self-amplifying RNA molecule. Self-amplifying RNA molecules are well known in the art and can be produced by using replication elements derived from viruses (e.g., alphavirus, flavivirus, picornavirus), and substituting the structural viral proteins with a nucleotide sequence encoding a protein of interest. A self-amplifying RNA molecule is typically a (+)-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as co-linear subgenomic transcripts, may be translated themselves to provide in situ expression of an encoded antigen, or may be transcribed to provide further transcripts with the same sense as the delivered RNA which are translated to provide in situ expression of the antigen. The overall results of this sequence of transcriptions is an amplification in the number of the introduced replicon RNAs and thereby the encoded antigen becomes a major polypeptide product of the cells.

Advantageously, the cell's translational machinery is used by self-amplifying RNA molecules to generate a significant increase of encoded gene products, such as proteins or antigens, which can accumulate in the cells or be secreted from the cells. Self-amplifying RNA molecules may, for example, stimulate toll-like receptors (TLR) 3, 7 and 8 and non TLR pathways (e.g., RIG-I, MD-5) by the products of RNA replication and amplification, and translation which may induce apoptosis of the transfected cell.

The self-amplifying RNA can, for example, contain at least one or more genes selected from the group consisting of viral replicases, viral proteases, viral helicases and other nonstructural viral proteins, and also comprise 5′- and 3′-end cis-active replication sequences, and if desired, heterologous sequences that encode a desired amino acid sequence (e.g., an antigen of interest). A subgenomic promoter that directs expression of the heterologous sequence can be included in the self-amplifying RNA. If desired, the heterologous sequence (e.g., an antigen of interest) may be fused in frame to other coding regions, with or without a ribosomal skipping peptide sequence in the self-amplifying RNA and/or may be under the control of an internal ribosome entry site (IRES).

In certain implementations, the self-amplifying RNA molecule is not encapsulated in a virus-like particle. Self-amplifying RNA molecules of the invention can be designed so that the self-amplifying RNA molecule cannot induce production of infectious viral particles. This can be achieved, for example, by omitting one or more viral genes encoding structural proteins that are necessary for the production of viral particles in the self-amplifying RNA. For example, when the self-amplifying RNA molecule is based on an alpha virus, such as Sindbis virus (SIN), Semliki forest virus and Venezuelan equine encephalitis virus (VEE), one or more genes encoding viral structural proteins, such as capsid (C) and/or envelope (E) glycoproteins, can be omitted.

If desired, self-amplifying RNA molecules of the invention can also be designed to induce production of infectious viral particles that are attenuated or virulent, or to produce viral particles that are capable of a single round of subsequent infection.

One suitable system for achieving self-amplification in this manner is to use an alphavirus-based replicon. Alphaviruses comprise a set of genetically, structurally, and serologically related arthropod-borne viruses of the Togaviridae family. Thirty-one species have been classified within the alphavirus genus, including, Sindbis virus, Semliki Forest virus, Ross River virus, chikungunya virus, and Venezuelan equine encephalitis virus. As such, the self-amplifying RNA of the invention may incorporate an RNA replicase derived from semliki forest virus (SFV), sindbis virus (SIN), Venezuelan equine encephalitis virus (VEE), Ross-River virus (RRV), eastern equine encephalitis virus, chikungunya virus, or other viruses belonging to the alphavirus genus.

An alphavirus-based “replicon” expression vector can be used in the invention. Replicon vectors may be utilized in several formats, including DNA, RNA, and recombinant replicon particles. Such replicon vectors have been derived from alphaviruses that include, for example, Sindbis virus (Xiong et al. (1989) Science 243:1188-1191; Dubensky et al., (1996) J. Virol. 70:508-519; Hariharan et al. (1998) J. Virol. 72:950-958; Polo et al. (1999) PNAS 96:4598-4603), Semliki Forest virus (Liljestrom (1991) Bio/Technology 9:1356-1361; Berglund et al. (1998) Nat. Biotech. 16:562-565), and Venezuelan equine encephalitis virus (Pushko et al. (1997) Virology 239:389-401). Alphaviruses-derived replicons are generally quite similar in overall characteristics (e.g., structure, replication), individual alphaviruses may exhibit some particular property (e.g., interferon sensitivity, and disease profile) that is unique. Therefore, chimeric alphavirus replicons made from divergent virus families may also be useful.

Alphavirus-based RNA replicons are typically (+)-stranded RNAs which lead to translation of a replicase (or replicase-transcriptase) after delivery to a cell. The replicase is translated as a polyprotein which auto-cleaves to provide a replication complex which creates genomic (−)-strand copies of the (+)-strand delivered RNA. These (−)-strand transcripts can themselves be transcribed to give further copies of the (+)-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc.

An RNA replicon can comprise, for example, an RNA genome from a picornavirus, togavirus (e.g., alphaviruses such as, for example, Sindbis virus, Semliki Forest virus, Venezuelan equine encephalitis virus, or Ross River virus), flavivirus (e.g., yellow fever virus), coronavirus, paramyxovirus, which has been modified by the replacement of one or more structural protein genes with a selected heterologous nucleic acid sequence encoding a product of interest.

In some aspects, a replicon will encode (i) a RNA-dependent RNA polymerase which can transcribe RNA from the replicon and (ii) an antigen. The polymerase can be, for example, an alphavirus replicase e.g., comprising one or more of alphavirus proteins nsP1, nsP2, nsP3 and nsP4. Whereas natural alphavirus genomes encode structural virion proteins in addition to the non-structural replicase polyprotein, in implementations the replicon does not encode alphavirus structural proteins. Thus, a replicon can lead to the production of genomic RNA copies of itself in a cell, but not to the production of RNA-containing virions. The inability to produce these virions means that, unlike a wild-type alphavirus, the replicon cannot perpetuate itself in infectious form. The alphavirus structural proteins which are necessary for perpetuation in wild-type viruses are absent from the replicon and their place is taken by gene(s) encoding the antigen of interest, such that the subgenomic transcript encodes the antigen rather than the structural alphavirus virion proteins.

A replicon useful with the invention can, for example, have two open reading frames. In one example, the first (5′) open reading frame encodes a replicase; the second (3′) open reading frame encodes an antigen. In some implementations the RNA may have additional (e.g., downstream) open reading frames e.g., to encode additional antigens or to encode accessory polypeptides.

A replicon can, for example, have a 5′ cap (e.g., a 7-methylguanosine), which often can enhance in vivo translation of the RNA. In some implementations the 5′ sequence of the replicon may need to be selected to ensure compatibility with the encoded replicase.

A replicon may have a 3′ poly-A tail. It may also include a poly-A polymerase recognition sequence (e.g., AAUAAA) near its 3′ end.

Replicons can have various lengths, but they are typically 5000-25000 nucleotides long e.g., 8000-15000 nucleotides, or 9000-12000 nucleotides.

The replicon can conveniently be prepared by in vitro transcription (IVT). IVT can use a (cDNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the replicon from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some implementations these requirements must be matched with the requirements of the encoded replicase, to ensure that the IVT-transcribed RNA can function efficiently as a substrate for its self-encoded replicase. Specific examples include Sindbis-virus-based plasmids (pSIN) such as pSINCP, described, for example, in U.S. Pat. Nos. 5,814,482 and 6,015,686, as well as in International Publication Nos. WO 97/38087, WO 99/18226 and WO 02/26209. The construction of such replicons, in general, is described in U.S. Pat. Nos. 5,814,482 and 6,015,686.

In other aspects, the self-amplifying RNA molecule is derived from or based on a virus other than an alphavirus, preferably, a positive-stranded RNA virus, a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

In other aspects, the self-amplifying RNA molecule is derived from or based on a replication competent virus (e.g., an oncolytic virus). An oncolytic virus preferentially infects and lyses (breaks down) cancer cells. As the infected cancer cells are destroyed, new infectious virus particles or virions are released, which can infect and destroy further cancer cells. Thus, oncolytic viruses not only cause direct destruction of cancer cells, but also stimulate host anti-cancer immune responses. In some implementations, the oncolytic virus may encode a tumor- or viral-associated antigen, neoantigen, and/or peptides. Suitable oncolytic viruses are known in the art and are available from sequence depositories, such as the American Type Culture Collection, Rockville, Md. Representative examples of suitable oncolytic viruses include, but are not limited to, poxvirus, adenovirus, adeno-associated virus, reovirus, retrovirus, senecavirus, measles, herpes simplex virus, Newcastle disease virus (NDV), vesicular stomatitis virus (VSV), mumps, influenza, Parvovirus, human hanta virus, myxoma virus, cytomegalovirus (CMV), lentivirus, coxsackievirus, echoviruses, Seneca Valley virus, Sindbis virus, JX-594, p53 expressing viruses, ONYX-15, Delta24, Telemelysin, Telomelysin-GFP, and vaccinia, and the like, and recombinant variants thereof. In some implementations, the oncolytic virus is genetically engineered for tumour selectivity. In other implementations, the oncolytic virus is naturally occurring. Naturally occurring oncolytic viruses include, but are not limited to, reovirus and senecavirus.

The self-amplifying RNA molecules of the invention are typically larger than other types of RNA (e.g., mRNA) that have been prepared using modified nucleotides. Typically, the self-amplifying RNA molecules of the invention contain at least about 3 kb. For example, the self-amplifying RNA can contain at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or more than 14 kb. In certain examples, the self-amplifying RNA is about 4 kb to about 14 kb, about 5 kb to about 14 kb, about 6 kb to about 14 kb, about 7 kb to about 14 kb, about 8 kb to about 14 kb, about 9 kb to about 14 kb, about 10 kb to about 14 kb, about 11 kb to about 14 kb, about 13 kb to about 14 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

The RNA molecules of the invention may comprise one or more types of modified nucleotides (e.g., pseudouridine, N6-methyladenosine, 5-methylcytidine, 5-methyluridine).

RNA molecule may encode a single heterologous polypeptide antigen or, optionally, two or more heterologous polypeptide antigens linked together in a way that each of the sequences retains its identity (e.g., linked in series) when expressed as an amino acid sequence. The heterologous polypeptides generated from the self-amplifying RNA may then be produced as a fusion polypeptide or engineered in such a manner to result in separate polypeptide or peptide sequences.

The RNA of the invention may encode one or more polypeptides. These polypeptides may consist of binding proteins, enzymes, cytokines, chemokines, hormones, or other functional proteins. Alternatively, these polypeptides may consist of antigens that contain a range of epitopes, such as epitopes capable of eliciting either a helper T-cell response, a cytotoxic T-cell response, an antibody response, or a combination thereof.

The RNA molecules described herein may be engineered to express multiple nucleotide sequences, from two or more open reading frames, thereby allowing co-expression of proteins, such as a two or more antibody sequences or two or more antigens together, or antigens together with cytokines or other immunomodulators, which can enhance the generation of an immune response. Such an RNA molecule might be particularly useful, for example, in the production of various gene products (e.g., proteins) at the same time, for example, as a two different single chain antibody sequences, heavy and light chain antibody sequences or multiple antigens to create a bivalent or multivalent vaccine.

The RNA molecules of the invention can be prepared using any suitable method. Several suitable methods are known in the art for producing RNA molecules that contain modified nucleotides. For example, a RNA molecule that contains modified nucleotides can be prepared by transcribing (e.g., in vitro transcription) a DNA that encodes the RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a RNA may be engineered, for example, to alter the stability of such RNA molecules, to increase resistance against RNases, to establish replication after introduction into appropriate host cells (“infectivity” of the RNA), and/or to induce or reduce innate and adaptive immune responses.

Suitable synthetic methods can be used alone, or in combination with one or more other methods (e.g., recombinant DNA or RNA technology), to produce a RNA molecule of the invention. Suitable methods for de novo synthesis are well-known in the art and can be adapted for particular applications. Illustrative methods include, for example, chemical synthesis using suitable protecting groups such as CEM, the β-cyanoethyl phosphoramidite method; and the nucleoside H-phosphonate method. These chemistries can be performed or adapted for use with automated nucleic acid synthesizers that are commercially available. Additional suitable synthetic methods are disclosed in Uhlmann et al. (1990) Chem Rev 90:544-84, and Goodchild J (1990) Bioconjugate Chem 1: 165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation and expression of nucleic acid sequences are known and conventional in the art.

The presence and/or quantity of one or more modified nucleotides in a RNA molecule can be determined using any suitable method. For example, a RNA can be digested to monophosphates (e.g., using nuclease P1) and dephosphorylated (e.g., using a suitable phosphatase such as CIAP), and the resulting nucleosides analyzed by reversed phase HPLC.

Optionally, the RNA molecules of the invention may include one or more modified nucleotides so that the RNA molecule will have less immunomodulatory activity upon introduction or entry into a host cell (e.g., a human cell) in comparison to the corresponding RNA molecule that does not contain modified nucleotides.

If desired, the RNA molecules can be screened or analyzed to confirm their therapeutic and prophylactic properties using various in vitro or in vivo testing methods that are known to those of skill in the art. For example, vaccines comprising RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a RNA molecule that encodes a polypeptide antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry after antigen stimulation.

RNA molecules that encode a polypeptide antigen can also be tested for ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays that can be used to characterize the self-amplifying RNA molecules of the invention can involve detecting expression of the encoded antigen by the target cells. For example, FACS can be used to detect antigen expression on the cell surface or intracellularly. Another advantage of FACS selection is that one can sort for different levels of expression; sometimes lower expression may be desired. Other suitable method for identifying cells which express a particular antigen involve panning using monoclonal antibodies on a plate or capture using magnetic beads coated with monoclonal antibodies.

B. DNA Molecules

In implementations where the bioactive agent is a DNA molecule, the DNA molecule may encode proteins of various types, including, without limitation, antigens, antibodies, toxins, growth factors, cytokines, and hormones. The DNA can include, without limitation, plasmid DNA, circular DNA, linear DNA, single-stranded DNA, modified DNA, antisense DNA, and aptamer DNA.

C. Antigens

The bioactive agent described herein can be a nucleic acid molecule (e.g., DNA or RNA) that encodes an antigen. Suitable antigens include, but are not limited to, a bacterial antigen, a viral antigen, a fungal antigen, a protazoan antigen, a plant antigen, a cancer antigen, or a combination thereto. The antigen can be involved in, or derived from, for example, an allergy, cancer, infectious disease, or auto-immune disease.

An antigen may be any target epitope, molecule (including a biomolecule), molecular complex (including molecular complexes that contain biomolecules), subcellular assembly, cell or tissue against which elicitation or enhancement of immunoreactivity in a subject is desired. Frequently, the term antigen will refer to a polypeptide antigen of interest. In certain implementations the antigen may be, or may be derived from, or may be immunologically cross-reactive with, an infectious pathogen and/or an epitope, biomolecule, cell or tissue that is associated with infection, cancer, autoimmune disease, allergy, asthma, or any other condition where stimulation of an antigen-specific immune response would be desirable or beneficial.

Certain implementations contemplate an antigen that is derived from at least one infectious pathogen such as a bacterium, a virus or a fungus, including an Actinobacterium such as M. tuberculosis or M. leprae or another mycobacterium; a bacterium such as a member of the genus Escherichia, Salmonella, Neisseria, Borrelia, Chlamydia, Clostridium or Bordetella; a virus such as a herpes simplex virus, a human immunodeficiency virus (HIV such as HIV-1 or HIV-2), an influenza virus, a parainfluenza virus, a measles virus, a mumps virus, a rubella virus, a coronavirus (such as SARS, MERS, or SARS-Cov-2), a rotavirus, a norovirus, a picorna virus (such as a poliovirus, an enterovirus, or a coxsacchie virus), a veterinary pathogen, for example, a feline immunodeficiency virus (FIV), cytomegalovirus, Varicella Zoster Virus, hepatitis virus, Epstein Barr Virus (EBV), a flavivirus virus (such as dengue virus, Japanese encephalitis virus, yellow fever virus, Zika virus, Powassan virus or tick-borne encephalitis virus), a henipah virus (such as hendra or nipah virus), a bunyavirus (such as Hantavirus or Rift Valley Fever virus), an arenavirus (such as lassa virus, junin virus, machupo virus, or guanarito virus), a filovirus (such as Ebola virus or Marburg virus), a lyssavirus (such as Rabies virus), respiratory syncytial virus, human papilloma virus (HPV) and a cytomegalovirus; a fungus such as Aspergillus, Blastomyces, Coccidioides and Pneumocystis or a yeast, including Candida species such as C. albicans, C. glabrata, C. krusei, C. lusitaniae, C. tropicalis and C. parapsilosis; a parasite such as a protozoan, for example, a Plasmodium species including P. falciparum, P. vivax, P. malariae and P. ovale; or another parasite such as one or more of Acanthamoeba, Entamoeba histolytica, Angiostrongylus, Schistosoma mansonii, Schistosoma haematobium, Schistosoma japonicum, Cryptosporidium, Ancylostoma, Entamoeba histolytica, Entamoeba coli, Entamoeba dispar, Entamoeba hartmanni, Entamoeba polecki, Wuchereria bancrofti, Giardia, Toxoplasma gondii, and Leishmania. In specific implementations, the antigen may be from, or related to antigens involved in tuberculosis, influenza, amebiasis, HIV, hepatitis, or Leishmaniasis.

In some implementations, the antigen is an influenza-related antigen. In some implementations, the antigen is an influenza-causing antigen. In some implementations, the antigen is from an influenza causing virus. In one implementation, the antigen comprises hemagglutinin (HA) from H5N1. In one implementation, the antigen comprises neuraminidase from H5N1.

For example, in certain implementations, antigens are derived from Borrelia sp., the antigens may include nucleic acid, pathogen derived antigen or antigenic preparations, recombinantly produced protein or peptides, and chimeric fusion proteins. One such antigen is OspA. The OspA may be a full mature protein in a lipidated form by virtue of its biosynthesis in a host cell (Lipo-OspA) or may alternatively be a non-lipidated derivative. Such non-lipidated derivatives include the non-lipidated NS1-OspA fusion protein which has the first 81 N-terminal amino acids of the non-structural protein (NS1) of the influenza virus, and the complete OspA protein, and another, MDP-OspA is a non-lipidated form of OspA carrying 3 additional N-terminal amino acids.

In certain implementations the antigen is derived from a virus such as from SARS-CoV-2 (spike protein), HIV-1, (such as tat, nef, gp120 or gp160), human herpes viruses, such as gD or derivatives thereof or Immediate Early protein such as ICP27 from HSV1 or HSV2, cytomegalovirus ((esp. Human)(such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, etc.), flaviviruses (e.g., dengue virus, Japanese encephalitis virus, yellow fever virus, Zika virus (such as prM or E), Poswanan virus, tick-borne encephalitis virus) or Influenza virus (whole live or inactivated virus, split influenza virus, grown in eggs or MDCK cells, or whole flu virosomes (as described by Gluck, Vaccine, 1992, 10, 915-920) or purified or recombinant proteins thereof, such as HA, NP, NA, PB1, PB2, PA, NS1 or M proteins, or combinations thereof).

In certain other implementations, the antigen is derived from one or more bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans: H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamentous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexneri; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or other bacterial pathogens.

In certain other implementations, the antigen is derived from one or more parasites (See, e.g., John, D. T. and Petri, W. A., Markell and Voge's Medical Parasitology-9th Ed., 2006, W B Saunders, Philadelphia; Bowman, D. D., Georgis' Parasitology for Veterinarians-8th Ed., 2002, W B Saunders, Philadelphia) such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; or from a helminth capable of infecting a vertebrate, such as: (i) nematode infections (including, but not limited to, Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, and Strongyloides stercoralis); (ii) trematode infections (including, but not limited to, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica); and (iii) cestode infections (including, but not limited to, Taenia saginata and Taenia solium). In certain implementations, the antigen is derived from Schistosoma spp., Schistosoma mansonii, Schistosoma haematobium, and/or Schistosoma japonicum, or derived from yeast such as Candida spp., including C. albicans; Cryptococcus spp., including C. neoformans.

Other specific antigens are derived from Chlamydia and include for example the High Molecular Weight Protein (HWMP) (WO 99/17741), ORF3 (EP 366 412), and putative membrane proteins (Pmps). Other Chlamydia antigens can be selected from the group described in WO 99128475. Certain antigens may be derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial vaccines comprise antigens derived from Haemophilus spp., including H. influenzae type B (for example PRP and conjugates thereof), non-typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Other specific antigens are derived from Hepatitis B. Derivatives of Hepatitis B Surface antigen are well known in the art and include, inter alia, those PreS1, PreS2, S antigens set forth described in European Patent applications EP-A414 374; EP-A-0304 578, and EP 198474.

In other implementations, the antigen is derived from the Human Papilloma Virus (HPV) considered to be responsible for genital warts (HPV 6 or HPV 11 and others), and the HPV viruses responsible for cervical cancer (HPV16, HPV18 and others). Particular antigens include L1 particles or capsomers, and fusion proteins comprising one or more antigens selected from the HPV 6 and HPV 11 proteins E6, E7, L1, and L2. Certain forms of fusion protein include L2E7 as disclosed in WO 96/26277, and protein D(1/3)-E7 disclosed in GB 9717953.5 (PCT/EP98/05285). Additional possible antigens include HPV 16, 18, 33, 58 antigens. For example, L1 or L2 antigen monomers, or L1 or L2 antigens presented together as a virus like particle (VLP) or the L1 alone protein presented alone in a VLP or capsomer structure. Such antigens, virus like particles and capsomer are per se known. See for example WO94/00152, WO94/20137, WO94/05792, and WO93/02184.

In other implementations, the antigen is a fusion protein. Fusion proteins may be included alone or as fusion proteins such as E7, E2 or F5 for example; particular implementations include a VLP comprising L1E7 fusion proteins (WO 96/11272). Particular HPV 16 antigens comprise the early proteins E6 or F7 in fusion with a protein D carrier to form Protein D-E6 or E7 fusions from HPV 16, or combinations thereof; or combinations of E6 or E7 with L2 (WO 96/26277). Alternatively, the HPV 16 or 18 early proteins E6 and E7, may be presented in a single molecule, for example a Protein D-E6/E7 fusion. Compositions may optionally contain either or both E6 and E7 proteins front HPV 18, for example in the form of a Protein D-E6 or Protein D-E7 fusion protein or Protein D E6/E7 fusion protein. Compositions may additionally comprise antigens from other HPV strains, for example from strains HPV 31 or 33.

Antigens may also be derived from parasites that cause Malaria. For example, antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No. PCT/EP92/02591, published as WO 93/10152 claiming priority from UK patent application No. 9124390.7. When expressed in yeast RTS is produced as a lipoprotein particle, and when it is co-expressed with the S antigen from HBV it produces a mixed particle known as RTS,S.

TRAP antigens are described in the International Patent Application No. PCT/GB89/00895 published as WO 90/01496. An implementation of the present invention is a Malaria vaccine wherein the antigenic preparation comprises a combination of the RTS,S and TRAP antigens. Other plasmodia antigens that are likely candidates to be components of a multistage Malaria vaccine are P. falciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27125, Pfs16, Pfs48/45, Pfs230 and their analogues in Plasmodium spp.

In one implementation, the antigen is derived from a cancer cell, as may be useful for the immunotherapeutic treatment of cancers. For example, the antigen may be a tumor rejection antigen such as those for prostate, breast, colorectal, lung, pancreatic, renal or melanoma cancers. Illustrative cancer or cancer cell-derived antigens include MAGE 1, 3 and MAGE 4 or other MAGE antigens such as those disclosed in WO99/40188, PRAME, BAGE, Lage (also known as NY Eos 1) SAGE and HAGE (WO 99/53061) or GAGE (Robbins and Kawakami, 1996 Current Opinions in Immunology 8, pp. 628-636; Van den Eynde et al., International Journal of Clinical & Laboratory Research (1997 & 1998); Correale et al. (1997), Journal of the National Cancer Institute 89, p. 293. These non-limiting examples of cancer antigens are expressed in a wide range of tumor types such as melanoma, lung carcinoma, sarcoma and bladder carcinoma. See, e.g., U.S. Pat. No. 6,544,518.

Other tumor-specific antigens include, but are not restricted to, tumor-specific or tumor-associated gangliosides such as GM2, and GM3 or conjugates thereof to carrier proteins; or a self peptide hormone such as whole length Gonadotrophin hormone releasing hormone (GnRH, WO 95/20600), a short 10 amino acid long peptide, useful in the treatment of many cancers. In another implementation prostate antigens are used, such as Prostate specific antigen (PSA), PAP, PSCA (e.g., Proc. Nat. Acad. Sci. USA 95(4) 1735-1740 1998), PSMA or, in one implementation an antigen known as Prostase. (e.g., Nelson, et al., Proc. Natl. Acad. Sci. USA (1999) 96: 3114-3119; Ferguson, et al. Proc. Natl. Acad. Sci. USA 1999. 96, 3114-3119; WO 98/12302; U.S. Pat. No. 5,955,306; WO 98/20117; U.S. Pat. Nos. 5,840,871 and 5,786,148; WO 00/04149. Other prostate specific antigens are known from WO 98/137418, and WO/004149. Another is STEAP (PNAS 96 14523 14528 7-12 1999).

Other tumor associated antigens useful in the context of the present invention include: Plu-1 (J Biol. Chem 274 (22) 15633-15645, 1999), HASH-1, HasH-2, Cripto (Salomon et al Bioessays 199, 21:61-70, U.S. Pat. No. 5,654,140) and Criptin (U.S. Pat. No. 5,981,215). Additionally, antigens particularly relevant for vaccines in the therapy of cancer also comprise tyrosinase and survivin.

In other implementations, the agents used in the compositions of the invention include antigens associated with respiratory diseases, such as those caused or exacerbated by bacterial infection (e.g., pneumococcal), for the prophylaxis and therapy of conditions such as chronic obstructive pulmonary disease (COPD). COPD is defined physiologically by the presence of irreversible or partially reversible airway obstruction in patients with chronic bronchitis and/or emphysema (Am J Respir Crit Care Med. 1995 November; 152(5 Pt 2):577-121). Exacerbations of COPD are often caused by bacterial (e.g., pneumococcal) infection (Clin Microbiol Rev. 2001 April; 14(2):336-63).

D. Antibody-Encoding Nucleic Acid

The bioactive agents described herein (e.g., RNA) may encode an antibody and/or antigen-binding fragment of an antibody, optionally operably linked to one or more expression control elements, such that delivery to a subject results in the production of said antibody or antigen-binding fragment in the subject. In some implementations, the bioactive agent may contain the coding sequence of the heavy chain and light chain in a single open reading frame. In other implementations, an NLC of the present invention may comprise two bioactive agents wherein one of the bioactive agents encodes a heavy chain whereas the other encodes a light chain. In other implementations, the bioactive agent may contain the coding sequence of the variable regions of the heavy and light chains linked by a short flexible polypeptide sequence such that the expressed biomolecule binds the antigen of interest. In some particular implementations, the produced antibody is capable of eliciting an immune response in an individual.

E. RNA Interference

In some implementations the bioactive polynucleotide associated with the NLC is a non-coding RNA such as an RNA interference (RNAi) polynucleotide. RNAi is a molecule capable of inducing RNA interference through interaction with the RNA interference pathway machinery of mammalian cells to degrade or inhibit translation of messenger RNA (mRNA) transcripts of a transgene in a sequence specific manner. Two primary RNAi polynucleotides are small (or short) interfering RNAs (siRNAs) and micro RNAs (miRNAs). RNAi polynucleotides may be selected from the group comprising: siRNA, microRNA, double-strand RNA (dsRNA), short hairpin RNA (shRNA), and expression cassettes encoding RNA capable of inducing RNA interference. siRNA comprises a double stranded structure typically containing 15-50 base pairs and preferably 21-25 base pairs and having a nucleotide sequence identical (perfectly complementary) or nearly identical (partially complementary) to a coding sequence in an expressed target gene or RNA within the cell. An siRNA may have dinucleotide 3′ overhangs. An siRNA may be composed of two annealed polynucleotides or a single polynucleotide that forms a hairpin structure.

MicroRNAs (miRNAs) are small noncoding RNA gene products about 22 nucleotides long that direct destruction or translational repression of their mRNA targets. If the complementarity between the miRNA and the target mRNA is partial, translation of the target mRNA is repressed. If complementarity is extensive, the target mRNA is cleaved. For miRNAs, the complex binds to target sites usually located in the 3′ UTR of mRNAs that typically share only partial homology with the miRNA. A “seed region”—a stretch of about seven (7) consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA.

F. CRISPR RNAs

In some implementations the NLC formulation comprises a synthetic short guide RNA (sgRNA) of the CRISPR/Cas9 genome editing thereby targeting a gene of interest. CRISPRs (Clustered Regularly Interspaced Short Palindromic Repeats) are loci containing multiple short direct repeats that are found in the genomes of approximately 40% of sequenced bacteria and 90% of sequenced archaea. CRISPR functions as a prokaryotic immune system, in that it confers resistance to exogenous genetic elements such as plasmids and phages. The CRISPR system provides a form of acquired immunity. Short segments of foreign DNA, called spacers, are incorporated into the genome between CRISPR repeats, and serve as a memory of past exposures. CRISPR spacers are then used to recognize and silence exogenous genetic elements in a manner analogous to RNAi in eukaryotic organisms. Cas9, an essential protein component in the Type II CRISPR/Cas9 system, forms an active endonuclease when complexed with two RNAs termed CRISPR RNA (crRNA) and trans-activating crRNA (tracrRNA), thereby slicing foreign genetic elements in invading phages or plasmids to protect the host cells.

The RNA-guided endonuclease based on CRISPR/Cas9 system been employed for eukaryotic genome editing. In certain implementations of the present invention, the bioactive agent is RNA that encodes sgRNAs and/or Cas9 endonucleases. In some implementations, the RNA comprises one or more polynucleotides encoding Cas9 and two guide RNAs, the first guide RNA comprising a spacer sequence that is complementary to a segment of the 5′ double-stranded break (DSB) locus, and the second guide RNA comprising a spacer sequence that is complementary to a segment of the 3′ DSB locus. Both guide RNAs may be provided as single-molecule guide RNAs (comprising tracrRNA and crRNA), or either or both may be provided as double-molecule guide RNAs comprising a crRNA and a tracrRNA that are not joined to each other but rather are separate molecules.

G. Polypeptides

In some implementations the one or more bioactive agents is a polypeptide. The polypeptide can be a full-length protein or a fragment thereof. In some implementations the polypeptide is a peptide. In some implementations, the polypeptide is a fusion protein. In some particular implementations, the fusion protein is capable of eliciting an immune response upon administration to an individual. In some implementations, the polypeptide is an antigen, as further described above. Polypeptides may be made by any suitable method known to one of skill in the art, including, for example, recombinant expression.

H. Small Molecules

In certain implementations, the present disclosure generally relates to a NLC composition where the one or more bioactive agents is a small molecule or therapeutic agent for drug delivery. A close association of drug molecule and the NLC may be influenced by drug physicochemical properties, surfactant type and concentration, lipid type, and production method. In certain implementations, the small molecule drug is encapsulated by the NLC, which is enabled by the liquid lipid phase component of the oil core that provides high drug solubility (Beloqui, A., et al. Nanomedicine 2016; 12(1): 143-161).

The NLC compositions provided herein may be suitable for drug delivery through various routes of administration, including, without limitation, dermal, transdermal, oral, intranasal, pulmonary, or ophthalmological routes of administration.

I. Hormones

In some implementations the one or more bioactive agents associated with the NLC is a polynucleotide or polypeptide that encodes a hormone or analog of a hormone. In some implementations, the NLC comprises a lipid that is conjugated to a hormone. The hormone may be selected from the group comprising human growth hormone, adrenocorticotropin, gonadotropin releasing hormone, oxytocin, leutinizing-hormone-releasing-hormone, follicle stimulating hormone, insulin, insulin-like growth factor, leptin, parathyroid hormone, thyroid stimulating hormone, or some combination thereof. In certain implementations the NLC formulation comprises a hormone or analog of a hormone in combination with a small molecule therapeutic compound as described above.

J. Adjuvants

In some implementations, the NLC is for vaccine delivery and one or more of the bioactive agents is an adjuvant or alternatively, the NLC compositions provided herein may be co-administered with an adjuvant. As used herein, the term adjuvant refers to a substance that enhances or potentiates an immune response. The immune response can be, for example, an antigen-specific immune response e.g., to an exogenous antigen.

Many adjuvants contain a substance designed to protect the antigen from rapid catabolism, such as aluminum hydroxide or mineral oil, and a stimulator of immune responses, such as lipid A (natural or synthetic). Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In some implementations, an adjuvant used in a composition described herein is a polysaccharide derived from bacteria or plants. Non-limiting examples of polysaccharide-based adjuvants that can be used alone or in combination with one or more additional adjuvant in a composition described herein include glucans (e.g., beta glucans), dextrans (e.g., sulfated and diethylaminoethyl-dextrans), glucomannans, galactomannans, levans, xylans, fructans (e.g., inulin), chitosan, endotoxins (e.g., lipopolysaccharide), biobran MGN-3, polysaccharides from Actinidia eriantha, eldexomer, and variations thereof.

Certain illustrative compositions employ adjuvant systems designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a compositions as provided herein, a patient may support an immune response that includes Th1- and Th2-type responses. Within an illustrative implementation, in which a response is predominantly Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989).

Certain adjuvants for use in eliciting a predominantly Th1-type response include, for example, a combination of monophosphoryl lipid A, for example 3-de-O-acylated monophosphoryl lipid A (3D-MPLTM), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present disclosure, for example combinations of at least two of the following group comprising QS21, QS7, Quil A, 0-escin, or digitonin.

Other illustrative adjuvants useful in the context of the disclosure include Toll-like receptor agonists, such as TLR1, TLR2, TLR3, TLR4, TLR5, TLR6, TLR7, TLR8, TLR7/8, TLR9 agonists, and the like. Still other illustrative adjuvants include imiquimod, gardiquimod, resiquimod, and related compounds.

In other implementations, the adjuvant is a glucopyranosyl lipid A (GLA) adjuvant, as described in U.S. Pat. No. 8,609,114 or 8,722,064. For example, in certain implementations, the TLR4 agonist is a synthetic GLA adjuvant.

In another implementation, an attenuated lipid A derivative (ALD) is incorporated into the compositions described herein. ALDs are lipid A-like molecules that have been altered or constructed so that the molecule displays lesser or different of the adverse effects of lipid A. These adverse effects include pyrogenicity, local Shwartzman reactivity and toxicity as evaluated in the chick embryo 50% lethal dose assay (CELD50). ALDs useful according to the present disclosure include monophosphoryl lipid A (MLA or MPL) and 3-deacylated monophosphoryl lipid A (3D-MLA or 3D-MPL). MLA (MPL) and 3D-MLA (3D-MPL) are known and need not be described in detail herein.

In the TLR4 agonist compounds above, the overall charge can be determined according to the functional groups in the molecule. For example, a phosphate group can be negatively charged or neutral, depending on the ionization state of the phosphate group.

### V. Methods of Making Illustrative Compositions Comprising Lyophilized Nanostructured Lipid Carriers

As provided herein, one method of making the NLCs described herein comprises (a) mixing the solid phase lipid, the liquid phase lipid, the cationic lipid, and the hydrophobic surfactant (e.g., sorbitan ester) to form an oil phase mixture; (b) mixing the hydrophilic surfactant and water to form an aqueous phase; and (c) mixing the oil phase mixture with the aqueous phase mixture to form the NLC. In an implementation, the solution containing NLC may contain a cake-forming excipient. The cake-forming excipient may be a saccharide such as, for example, sucrose or trehalose. In some implementations, a further step comprises combining one or more bioactive agents with the NLC such that the bioactive agents associate with the surface of the NLC by non-covalent interactions or by reversible covalent interactions. Such implementations are possible where the bioactive agent is negatively charged, such as an RNA molecule or a DNA molecule. The negative charges on the bioactive agent interact with the cationic lipid in the NLC, thereby associating the negatively charged bioactive agent with the NLC. Nucleotides may complex with the NLC at a N/P ratio of about 0.1 to about 750. In some implementations, the N/P ratio may be about 5-20 such as about 15. The N/P ratio is the ratio of positively-chargeable polymer amine (N=nitrogen) groups to negatively-charged nucleic acid phosphate (P) groups. In other implementations, where the bioactive agent is hydrophobic, it is combined with the components in step (a) to form part of the oil phase mixture and be contained within the lipid core of the NLC. In some implementations, the bioactive agent may be attached to a component of the surface of the NLC via covalent interactions. A solution containing the bioactive agent may contain a cake-forming excipient. The cake-forming excipient may be a saccharide such as, for example, sucrose or trehalose.

Mixing the solid phase lipid, the liquid phase lipid, the cationic lipid, and the hydrophobic surfactant (e.g., sorbitan ester) to form an oil phase mixture may be achieved, for example, by heating and sonication. Mixing the oil phase mixture with the aqueous phase mixture may be achieved, for example, by various emulsification methods, including, without limitation, high shear emulsification and microfluidization.

The NLC with any bioactive agent(s) if added is lyophilized using techniques known to those of ordinary skill in the art for lyophilizing vaccine or pharmaceutical compositions. The lyophilization process consists of freezing a solution and then putting it under vacuum to draw off the frozen water by sublimation. In an implementation, the concentration of cake-forming excipient may be adjusted prior to lyophilization. For example, the concentration of cake-forming excipient may be adjusted to 10-20% w/v of the solution, such as about 20% w/v of the solution, prior to lyophilization. The concentration may be adjusted by addition of cake-forming excipient.

A. Characteristics of the Lyophilized Nanostructured Lipid Carriers

In one aspect, the desired thermostability characteristics of the thermostable lyophilized vaccine NLC is that the lyophilized composition should possess certain desirable characteristics including: long-term stability at refrigerated or room temperature; short reconstitution time; maintenance of the cake appearance after storage equivalent to the cake appearance immediately after lyophilization; protection of integrity and activity of any bioactive agent; and consistent particle size before and after lyophilization.

In one implementation, a thermostable cake as used herein refers to a cake produced from a single vial lyophilization of the NLC of the invention that may comprise a bioactive agent and/or adjuvants in the presence of one or more suitable cake-forming excipients that when stored or exposed through storage or transport for several months to temperatures of about 4° C. or about 25° C. maintains the desirable characteristics.

B. Assessment of Thermostability

Thermostability of the lyophilized vaccine compositions provided herein can be assessed in the lyophilized state or following reconstitution. Thermostability of the lyophilized vaccine compositions provided herein can be assessed by visual observation, and/or with the aid of one or more assays provided herein. These assays can provide an estimate of the integrity of the NLC and any bioactive agent following lyophilization and reconstitution. The thermostability assays and observations described herein can be carried out at any time point including, for example, upon lyophilization, 2 weeks following lyophilization, 5 weeks following lyophilization, 3 months following lyophilization, 6 months following lyophilization, 8, months following lyophilization, 12 months following lyophilization, 21 months following lyophilization or beyond. Prior to carrying out the assays and observations, the lyophilized composition can be maintained, stored at, or exposed to temperatures of about −80° C., −20° C., 4° C., 25° C., or 40° C.

In some implementations, the thermostability of the lyophilized vaccine compositions provided herein is assessed by visual observation, prior to reconstitution. In some implementations, the thermostability of the lyophilized vaccine compositions provided herein is assessed by visual observation, following reconstitution. In other implementations, the thermostability of the lyophilized vaccine compositions provided herein is assessed following reconstitution by the aid of one or more assays, for example biophysical, biochemical, and/or biological assays.

In one implementation, the lyophilized cake resulting upon lyophilization of the NLC formulation, can be observed for color and consistency. Thermostability may be determined by the cake maintaining size, structure, and color. In some implementations, the cake referred to herein is a porous and spongy structure-like material resulting from the lyophilization process; or the cake is the solid content remaining after the freeze-drying process. In some implementations, the cake's appearance can be described as a spongiform cake, lovely cake, and elegant cake. “Elegant cake” as used in the field of lyophilized formulations refers to the visual appearance of a lyophilized cake that is uniform in appearance, free from residues, and discoloration. (See S. M. Patel et al., Lyophilized Drug Product Cake Appearance: What Is Acceptable?, J. Pharm Sci, Vol. 106(7), 2017, pages 1706-1721.) In some implementations, a cake can be visually inspected for lack of cracking, collapse (also can be described as shrinking or pulling away from the sides of the vial, depression or slight indentation of the top of cake, or a decrease in total volume of the cake), and/or a change in coloration or discoloration such as browning or yellowing of the cake. In some implementations the cake can be classified as an elegant cake, a white cake, an elegant white cake, a spongiform white cake, a white cake with increased volume, a yellow cake, a yellowing cake, a brown cake, a browning cake, or a shrinking/shrunk cake. In some implementations, discoloration or browning as used herein refers to a formulation which contains reducing sugars (for example sucrose) which upon lyophilization and storage of the cake can undergo a Maillard reaction or reduction of the sugars resulting in a discoloration of the original cake resulting in visually a yellow-to-brown to tint to the cake.

In some implementations, if no cake forms upon lyophilization, the resulting composition can be characterized as a clear film, a thin film, a thick white film, or solidified bubbles. In some implementations, desired cakes of the invention refer to cakes that after exposure, storage, or maintenance of the cake at temperatures of 4° C. or about 25° C. display the characteristics of a freshly lyophilized cake. (“Excipients used in lyophilization of small molecules” Ankit Bahetia, Lokesh Kumarb, Arvind K. Bansal, J. Excipients and Food Chem. 1 (1) 2010; 41-54.)

In some implementations, the emulsion particle size is evaluated following reconstitution of the lyophilized composition. For example, dynamic light scattering (DLS) can be used to evaluate emulsion particle size. In some implementations, this is compared to the emulsion particle size prior to lyophilization, for example in the liquid stable emulsion state prior to lyophilization. In some implementations the emulsion particle size is not compared to the particles size prior to lyophilization. In some implementations herein, the particle size is determined by measuring the hydrodynamic diameter or Z-average diameter (Z-Ave d) of the liquid lyophilized composition. In particular implementations, a thermostable composition is indicated when the reconstituted liquid emulsion of the lyophilized composition stored for at least 8 months at about 25° C. or for at least 21 months at about 4° C. has a particle size that increases less than about 20%, less than about 15%, less than about 10%, or less than about 5%. In particular implementations, the reconstituted vaccine has a particle size with a Z-average diameter range of about 100 nm to about 200 nm, a Z-average diameter range of about 150 nm, or a Z-average diameter range of about 125 nm.

In some implementations, creaming of the emulsion is evaluated following reconstitution of the lyophilized composition.

In some implementations, reverse phase high performance liquid chromatography (RP-HPLC) is used to evaluate the chemical degradation, if any, of the components. In one illustrative implementation, the chemical degradation of squalene, DOTAP, and trimyristin, is monitored by RP-HPLC. A thermostable composition as provided herein is one that exhibits no more than or about 50%, 40%, 30%, 20%, 15%, 10%, 9%, 8%, 7%, 6%, 5%, 4%, 3%, 2%, 1% component degradation, loss, or breakdown after reconstitution of the thermostable lyophilized composition following long-term storage at a temperature of about 4° C. or about 25° C. A highly thermostable composition is one that exhibits no more than about 20% component degradation, loss, or breakdown under the above conditions.

In some implementations, thermostability is assessed by evaluating reconstitution of the cakes following lyophilization. The cakes may be reconstituted in water such as nuclease free water. The cakes may be reconstituted in a liquid other than water. In implementations, the cakes reconstitute in less than 5 minutes, less than 4 minutes, less than 3 minutes, less than 2 minutes, or less than 1 minute. Desired cakes have an appearance as identified by visual inspection following lyophilization that is similar or the same as the appearance of the emulsion prior to lyophilization. In implementations, upon reconstitution the lyophilized cake forms a milky white solution. Desired cakes have a viscosity following reconstitution similar or the same as the viscosity prior to lyophilization. Desired cakes are free of residual precipitates following lyophilization. In some implementations, the cakes may reconstitute with gentle mixing. In some implementations, cakes may reconstitute with rigorous vortexing. In some implementations, the cakes may not reconstitute even with rigorous vortexing.

C. Thermostability Characteristics

In one aspect, the lyophilized NLC compositions provided herein are thermostable at about 4° C., or at about 25° C., or at about 40° C. In one aspect, the lyophilized NLC compositions provided herein are thermostable at temperatures at or below 4° C. for at least 21 month, 12 months, 8 months, 6 months, 3 months, 5 weeks, or 2 weeks. In one aspect, the lyophilized NLC compositions provided herein are thermostable at temperatures at or below 25° C. for at least 8 months, 6 months, 3 months, 5 weeks, or 2 weeks. In one aspect, the lyophilized NLC compositions provided herein are thermostable at temperatures at or below 40° C. for at least 5 weeks or 2 weeks.

### VI. Compositions Comprising the Lyophilized Nanostructured Lipid Carriers

Provided herein are formulations, compositions, and pharmaceutical compositions comprising the lyophilized NLC compositions described herein.

The compositions comprising the NLC and bioactive agent can optionally further comprise a pharmaceutically acceptable carrier, excipient, or diluent.

The compositions described herein can be administered to a subject for any vaccination, therapeutic or diagnostic purposes.

Provided here are pharmaceutical compositions comprising the presently disclosed compositions further in combination with a pharmaceutically acceptable carrier, excipient or diluent.

In some implementations provided herein, the NLC and pharmaceutical compositions provided herein capable of being filtered through a 0.45-micron filter. In some implementations, the pharmaceutical composition is capable of being filtered through a 0.22-micron filter. In some implementations, the pharmaceutical composition is capable of being filtered through a 0.20-micron filter.

In one implementation, the present invention is drawn to a pharmaceutical composition comprising a composition comprising an NLC and an associated bioactive agent. Such a composition may be administered to a subject in order to stimulate an immune response, e.g., a non-specific immune response or an antigen-specific immune response, for the purpose of diagnosis, treating or preventing a disease or other condition, such as an infection by an organism.

In some other implementations, the pharmaceutical composition is a vaccine composition that comprises the compositions described herein in combination with a pharmaceutically acceptable carrier, excipient, or diluent. Illustrative carriers are usually nontoxic to recipients at the dosages and concentrations employed.

In some aspects, the pharmaceutical compositions provided herein are administered to a subject to generate a response in the subject, for example, for generating an immune response in the subject. Typically, a therapeutically effective amount is administered to the subject.

The term “effective amount” or “therapeutically effective amount” refers to an amount that is sufficient to achieve or at least partially achieve the desired effect, e.g., sufficient to generate the desired immune response. An effective amount of a NLC or pharmaceutical composition is administered in an “effective regime.” The term “effective regime” refers to a combination of amount of the composition being administered and dosage frequency adequate to accomplish the desired effect.

Actual dosage levels may be varied so as to obtain an amount that is effective to achieve a desired response for a particular patient, composition, and mode of administration, without being toxic to the patient. The selected dosage level will depend upon a variety of pharmacokinetic factors in combination with the particular compositions employed, the age, sex, weight, condition, general health and prior medical history of the subject being treated, and like factors well-known in the medical arts.

In illustrative therapeutic implementations provided herein, a dosage of about 1 μg/kg to about 10 mg/kg of a therapeutic pharmaceutical composition is administered. It will be evident to those skilled in the art that the number and frequency of administrations will be dependent upon the response of the subject.

In illustrative vaccine-based implementations provided herein, about 1 μg-100 μg of the antigen or 0.1 μg-10 mg of the nucleic acid encoding the antigen will be administered per administration. Illustrative formulations of the present permit a human dose of from about 0.1 ug, about 1 ug, about 5 μg or about 10 ug to about 500 μg of replicon RNA. Illustrative formulations of the present permit a human dose of about 5 μg to about 20 μg replicon RNA.

It will be evident to those skilled in the art that the number and frequency of administrations will be dependent upon the response of the subject. Illustrative formulations allow for therapeutic efficacy after as little as one immunization.

“Pharmaceutically acceptable carriers” for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

The pharmaceutical compositions may be in any form which allows for the composition to be administered to a patient. For example, the composition may be in the form of a solid, liquid or gas (aerosol). Typical routes of administration include, without limitation, oral, topical, parenteral, sublingual, buccal, rectal, vaginal, intravenous, intradermal, transdermal, intranasal, intramucosal, pulmonary or subcutaneous. The term parenteral as used herein includes iontophoretic, sonophoretic, thermal, transdermal administration and also subcutaneous injections, intravenous, intramuscular, intrasternal, intracavernous, intrathecal, intrameatal, intraurethral injection or infusion techniques. In some implementations, a composition as described herein (including vaccine and pharmaceutical compositions) is administered intradermally by a technique selected from iontophoresis, microcavitation, sonophoresis, jet injection, or microneedles. In one implementation, a composition as described herein is administered intradermally using the microneedle device manufactured by NanoPass Technologies Ltd., Nes Ziona, Israel, e.g., MicronJet600 (see, e.g., U.S. Pat. Nos. 6,533,949 and 7,998,119 and Yotam, et al., Human vaccines & immunotherapeutics 11(4): 991-997 (2015).

In certain implementations, the compositions of the present disclosure may be delivered by intranasal sprays, inhalation, and/or other aerosol delivery vehicles. Methods for delivering genes, polynucleotides, and peptide compositions directly to the lungs via nasal aerosol sprays has been described e.g., in Southam et al., Distribution of intranasal instillations in mice: effects of volume, time, body position, and anesthesia, Am J Physiol Lung Cell Mol Physiol, Volume 282, 2002, pages L833-L839, U.S. Pat. Nos. 5,756,353 and 5,804,212. Likewise, the delivery of drugs using intranasal microparticle resins (Takenaga et al., Microparticle resins as a potential nasal drug delivery system for insulin, Journal of Controlled Release, Volume 52, Issues 1-2, 1998, Pages 81-87) and lysophosphatidyl-glycerol compounds (U.S. Pat. No. 5,725,871) are also well-known in the pharmaceutical arts. Likewise, transmucosal drug delivery in the form of a polytetrafluoroetheylene support matrix is described in U.S. Pat. No. 5,780,045.

The pharmaceutical composition can be formulated so as to allow the active ingredients contained therein to be bioavailable upon administration of the composition to a subject. Compositions that will be administered to a subject take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, compositions can contain one or more of a sweetening agent, preservatives, dye/colorant and flavor enhancer. In a composition intended to be administered by injection by needle and syringe or needle free jet injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose.

In another implementation, a composition of the present disclosure is formulated in a manner which can be aerosolized.

It may also be desirable to include other components in a pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL-12, GM-CSF, gamma interferon and IL-12.

While any suitable carrier known to those of ordinary skill in the art may be employed in the pharmaceutical compositions of the present disclosure, the type of carrier will vary depending on the mode of administration and whether a sustained release is desired. For parenteral administration, such as subcutaneous injection, the carrier can comprise water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 microns.

Pharmaceutical compositions may also contain diluents such as buffers, antioxidants such as ascorbic acid, polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are illustrative appropriate diluents. For example, a product may be formulated as a lyophilizate using appropriate excipient solutions (e.g., sucrose) as diluents.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the antigen (e.g., GLA-antigen vaccine composition) or GLA (e.g., immunological adjuvant composition; GLA is available from Avanti Polar Lipids, Inc., Alabaster, AL; e.g., product number 699800) of from about 0.1 to about 10% w/v (weight per unit volume).

The composition may be intended for rectal administration, in the form, e.g., of a suppository which can melt in the rectum and release the drug. The composition for rectal administration may contain an oleaginous base as a suitable nonirritating excipient. Such bases include, without limitation, lanolin, cocoa butter and polyethylene glycol. In the methods of the invention, the pharmaceutical compositions/adjuvants may be administered through use of insert(s), bead(s), timed-release formulation(s), patch(es) or fast-release formulation(s).

Optionally, to control tonicity, the NLC may comprise a physiological salt, such as a sodium salt. Sodium chloride (NaCl), for example, may be used at about 0.9% (w/v) (physiological saline). Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the presently disclosed compositions. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the presently disclosed compositions. Non-ionic tonicity modifying agents can be present at a concentration of from about 0.1% to about 10% or about 1% to about 10%, depending upon the agent that is used. If NLCs are formulated for parenteral administration, it is preferable to make the osmolarity of the NLC composition the same as normal physiological fluids, preventing post-administration consequences, such as post-administration swelling or rapid absorption of the composition.

Optionally, NLCs may be formulated with cryoprotectants comprising, Avicel PH102 (microcrystalline cellulose), Avicel RC591 (mixture of microcrystalline cellulose and sodium carboxymethyl cellulose), Mircrocelac® (mixture of lactose and Avicel), or a combination thereof. Optionally, NLCs may be formulated with a preservative agent such as, for example, Hydrolite 5.

### VII. Methods of Using the Compositions of the Present Disclosure

A. Therapeutics

In some implementations the agent is useful for therapeutic purposes. Thus, in some implementations, the compositions described comprise the NLCs provided herein, and further comprise a bioactive agent for the treatment of a disease, condition, or disorder.

In some implementations the bioactive agent is useful for the treatment or prevention of allergy, cancer, infectious disease, autoimmunity, or addiction. In some implementations the bioactive agent is useful for the stimulating, enhancing and/or modulating an immune response.

In some aspects of the disclosed implementations, the compositions comprise cancer antigens or nucleic acids encoding a cancer antigen. In some implementations, a vaccine composition comprises a cancer antigen will be useful against any cancer characterized by tumor associated antigen expression, such as HER-2/neu expression or other cancer-specific or cancer-associated antigens.

Compositions and methods according to certain implementations of the present disclosure may also be used for the prophylaxis or therapy of autoimmune diseases, which include diseases, conditions or disorders wherein a host's or subject's immune system detrimentally mediates an immune response that is directed against “self” tissues, cells, biomolecules (e.g., peptides, polypeptides, proteins, glycoproteins, lipoproteins, proteolipids, lipids, glycolipids, nucleic acids such as RNA and DNA, oligosaccharides, polysaccharides, proteoglycans, glycosaminoglycans, or the like, and other molecular components of the subjects cells and tissues) or epitopes (e.g., specific immunologically defined recognition structures such as those recognized by an antibody variable region complementarity determining region (CDR) or by a T cell receptor CDR.

Autoimmune diseases are thus characterized by an abnormal immune response involving either cells or antibodies that are in either case directed against normal autologous tissues. Autoimmune diseases in mammals can generally be classified in one of two different categories: cell-mediated disease (i.e., T-cell) or antibody-mediated disorders. Non-limiting examples of cell-mediated autoimmune diseases include multiple sclerosis, rheumatoid arthritis, Hashimoto thyroiditis, type I diabetes mellitus (Juvenile onset diabetes) and autoimmune uvoretinitis. Antibody-mediated autoimmune disorders include, but are not limited to, myasthenia gravis, systemic lupus erythematosus (or SLE), Graves' disease, autoimmune hemolytic anemia, autoimmune thrombocytopenia, autoimmune asthma, cryoglobulinemia, thrombic thrombocytopenic purpura, primary biliary sclerosis and pernicious anemia. The antigen(s) associated with: systemic lupus erythematosus is small nuclear ribonucleic acid proteins (snRNP); Graves' disease is the thyrotropin receptor, thyroglobulin and other components of thyroid epithelial cells; pemphigus is cadherin-like pemphigus antigens such as desmoglein 3 and other adhesion molecules; and thrombic thrombocytopenic purpura is antigens of platelets.

The compositions provided herein may be used for inducing protective immunity, for example against viruses include the use of polypeptides that contain at least one immunogenic portion of one or more viral proteins and DNA and/or RNA molecules encoding such polypeptides. In addition, such compounds may be formulated into vaccines and/or pharmaceutical compositions for immunization against viral infection.

In other implementations, the compositions of the present disclosure include antigens associated with respiratory diseases, such as those caused or exacerbated by bacterial infection (e.g., pneumococcal), for the prophylaxis and therapy of conditions such as chronic obstructive pulmonary disease (COPD).

In addition to direct in vivo procedures, ex vivo procedures may be used in which cells are removed from a host, modified, and placed into the same or another host animal. It will be evident that one can utilize any of the compositions noted above for introduction of antigen-encoding nucleic acid molecules into tissue cells in an ex vivo context. Protocols for viral, physical and chemical methods of uptake are well known in the art.

In some implementations, the compositions of the present disclosure are used to boost or enhance an immune response in a subject. In some such implementations, the bioactive agent is an adjuvant. Nonlimiting illustrative adjuvants include TLR agonists (including TLR2, TLR3, TLR4, TLR7, TLR8, and TLR9 agonists), Rig-I agonists, saponins, carbohydrates, carbohydrate polymers, conjugated carbohydrates, whole viral particles, virus-like particles, viral fragments, and cellular fragments. Examples of such adjuvants include, but are not limited to, double-stranded RNA, RIBOXXOL, poly(I:C), and Hiltonol®. In some implementations, the composition comprises a stable emulsion and/or a nanostructured lipid carrier. In some implementations, the composition comprises a stable emulsion and/or a nanostructured lipid carrier that comprises squalene.

In some aspects, the compositions of the present disclosure are useful for enhancing or eliciting, in a host, a patient or in cell culture, an immune response. As used herein, the term “subject” refers to any vertebrate. A patient may be afflicted with an infectious disease, cancer, such as breast cancer, or an autoimmune disease, or may be normal (i.e., free of detectable disease and/or infection). A “cell culture” is any preparation containing immunocompetent cells or isolated cells of the immune system (including, but not limited to, T cells, macrophages, monocytes, B cells and dendritic cells). Such cells may be isolated by any of a variety of techniques well known to those of ordinary skill in the art (e.g., Ficoll-hypaque density centrifugation). The cells may (but need not) have been isolated from a patient afflicted with cancer and may be reintroduced into a patient after treatment.

B. Vaccine

The present disclosure thus provides compositions for altering (i.e., increasing or decreasing in a statistically significant manner, for example, relative to an appropriate control as will be familiar to persons skilled in the art) immune responses in a host capable of mounting an immune response. As will be known to persons having ordinary skill in the art, an immune response may be any active alteration of the immune status of a host, which may include any alteration in the structure or function of one or more tissues, organs, cells or molecules that participate in maintenance and/or regulation of host immune status. Typically, immune responses may be detected by any of a variety of well-known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); or any other criterion by which the presence of an immune response may be detected.

Determination of the induction of an immune response by the compositions of the present disclosure may be established by any of a number of well-known immunological assays with which those having ordinary skill in the art will be readily familiar. Such assays include, but need not be limited to, in vivo or in vitro determination of: soluble antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are widely known and may be found, for example in Lefkovits (Immunology Methods Manual: The Comprehensive Sourcebook of Techniques, 1998; see also Current Protocols in Immunology; see also, e.g., Weir, Handbook of Experimental Immunology, 1986 Blackwell Scientific, Boston, MA; Mishell and Shigii (eds.) Selected Methods in Cellular Immunology, 1979 Freeman Publishing, San Francisco, CA; Green and Reed, 1998 Science 281:1309 and references cited therein).

Detection of the proliferation of antigen-reactive T cells may be accomplished by a variety of known techniques. For example, T cell proliferation can be detected by measuring the rate of DNA synthesis, and antigen specificity can be determined by controlling the stimuli (such as, for example, a specific desired antigen or a control antigen-pulsed antigen presenting cells) to which candidate antigen-reactive T cells are exposed. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a particular antigen may be quantified.

Detection of antigen-specific antibody production may be achieved, for example, by assaying a sample (e.g., an immunoglobulin containing sample such as serum, plasma, or blood) from a host treated with a vaccine according to the present disclosure using in vitro methodologies such as radioimmunoassay (RIA), enzyme linked immunosorbent assays (ELISA), equilibrium dialysis, solid phase immunoblotting including Western blotting, plaque-reduction neutralization test (PRNT), or pseudovirus neutralization assay. In implementations ELISA assays may further include antigen-capture immobilization of the target antigen with a solid phase monoclonal antibody specific for the antigen, for example, to enhance the sensitivity of the assay. Elaboration of soluble mediators (e.g., cytokines, chemokines, lymphokines, prostaglandins, etc.) may also be readily determined by enzyme-linked immunosorbent assay (ELISA), for example, using methods, apparatus and reagents that are readily available from commercial sources (e.g., Sigma, St. Louis, MO; see also R & D Systems 2006 Catalog, R & D Systems, Minneapolis, MN).

Any number of other immunological parameters may be monitored using routine assays that are well known in the art. These may include, for example, antibody dependent cell-mediated cytotoxicity (ADCC) assays, flow cytometry detection of antigen-specific T cell responses, secondary in vitro antibody responses, flow immunocytofluorimetric analysis of various peripheral blood or lymphoid mononuclear cell subpopulations using well established marker antigen systems, immunohistochemistry or other relevant assays. These and other assays may be found, for example, in Rose et al. (Eds.), Manual of Clinical Laboratory Immunology, 5th Ed., 1997 American Society of Microbiology, Washington, DC.

Accordingly, it is contemplated that the compositions provided herein will be capable of eliciting or enhancing in a host at least one immune response that is selected from a Th1-type T lymphocyte response, a TH2-type T lymphocyte response, a cytotoxic T lymphocyte (CTL) response, an antibody response, a cytokine response, a lymphokine response, a chemokine response, and an inflammatory response. In certain implementations the immune response may comprise at least one of production of one or a plurality of cytokines wherein the cytokine is selected from interferon-gamma (IFN-γ), tumor necrosis factor-alpha (TNF-α), production of one or a plurality of interleukins wherein the interleukin is selected from IL-1, IL-2, IL-3, IL-4, IL-6, IL-8, IL-10, IL-12, IL-13, IL-16, IL-18 and IL-23, production one or a plurality of chemokines wherein the chemokine is selected from MIP-1a, MIP-1β, RANTES, CCL2, CCL4, CCL5, CXCL1, and CXCL5, and a lymphocyte response that is selected from a memory T cell response, a memory B cell response, an effector T cell response, a cytotoxic T cell response and an effector B cell response.

### VIII. Methods of Generating an Immune Response

Provided herein are methods of generating an immune response in a subject, including the step of administering to a subject in need thereof a therapeutically effective amount of a composition described herein, where the bioactive agent is a protein antigen or a nucleic acid molecule encoding a protein antigen. In illustrative implementations, the bioactive agent is an RNA (e.g., mRNA or saRNA) or a DNA molecule encoding a protein antigen. In some implementations, methods of boosting or enhancing an immune response are provided, wherein the bioactive agent is an adjuvant.

Typical routes of administration of the therapeutically effective amount of the composition include, without limitation, oral, topical, parenteral, sublingual, buccal, rectal, vaginal, intravenous, intradermal, transdermal, intranasal, intramucosal, or subcutaneous (s.c.). In some illustrative implementations, administration of the composition is intramuscular (i.m.), ocular, parenteral, or pulmonary.

In illustrative implementations, the compositions disclosed herein are vaccine compositions and are used as vaccines. The compositions described herein can be used for generating an immune response in the subject (including a non-specific response and an antigen-specific response). In some implementations, the immune response comprises a systemic immune response. In some implementations, the immune response comprises a mucosal immune response. Generation of an immune response includes stimulating an immune response, boosting an immune response, or enhancing an immune response.

The compositions described herein may be used to enhance protective immunity against a virus. Such viruses and viral antigens include, for example, corona viruses (such as SARS, MERS, and SARS-CoV-2), HIV-1, (such as tat, nef, gp120 or gp160), human herpes viruses (such as gD or derivatives thereof or Immediate Early protein such as ICP27 from HSV1 or HSV2), cytomegalovirus ((esp. Human, such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, etc.), flaviviruses (e.g., dengue virus, Japanese encephalitis virus, yellow fever virus, Zika virus, Poswanan virus, tick-borne encephalitis virus) or Influenza virus (whole live or inactivated virus, split influenza virus, grown in eggs or MDCK cells, or whole flu virosomes (as described by Reinhard Glück, Immunopotentiating reconstituted influenza virosomes (IRIVs) and other adjuvants for improved presentation of small antigens, Vaccine, Volume 10, Issue 13, 1992, Pages 915-919) or purified or recombinant proteins thereof, such as HA, NP, NA, or M proteins, or combinations thereof).

The compositions described herein may be used to enhance protective immunity against one or more bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans: H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamentous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexneri; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or other bacterial pathogens.

The compositions described herein may be used to enhance protective immunity against one or more parasites (See, e.g., John, D. T. and Petri, W. A., Markell and Voge's Medical Parasitology-9th Ed., 2006, W B Saunders, Philadelphia; Bowman, D. D., Georgis' Parasitology for Veterinarians-8th Ed., 2002, W B Saunders, Philadelphia) such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; or from a helminth capable of infecting a mammal, such as: (i) nematode infections (including, but not limited to, Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, and Strongyloides stercoralis); (ii) trematode infections (including, but not limited to, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica); and (iii) cestode infections (including, but not limited to, Taenia saginata and Taenia solium). In certain implementations, the antigen is derived from Schistosoma spp., Schistosoma mansonii, Schistosoma haematobium, and/or Schistosoma japonicum, or derived from yeast such as Candida spp., including C. albicans; Cryptococcus spp., including C. neoformans. infectious pathogen such as a bacterium, a virus or a fungus, including an Actinobacterium such as M. tuberculosis or M. leprae or another mycobacterium; a bacterium such as a member of the genus Salmonella, Neisseria, Borrelia, Chlamydia or Bordetella; a virus such as a herpes simplex virus, a human immunodeficiency virus (HIV), a feline immunodeficiency virus (FIV), cytomegalovirus, Varicella Zoster Virus, hepatitis virus, Epstein Barr Virus (EBV), Zika virus (ZIKV) respiratory syncytial virus, human papilloma virus (HPV) and a cytomegalovirus; HIV such as HIV-1 or HIV-2; a fungus such as Aspergillus, Blastomyces, Coccidioides and Pneumocystis or a yeast, including Candida species such as C. albicans, C. glabrata, C. krusei, C. lusitaniae, C. tropicalis and C. parapsilosis; a parasite such as a protozoan, for example, a Plasmodium species including P. falciparum, P. vivax, P. malariae and P. ovale; or another parasite such as one or more of Acanthamoeba, Entamoeba histolytica, Angiostrongylus, Schistosoma mansoni, Schistosoma haematobium, Schistosoma japonicum, Cryptosporidium, Ancylostoma, Entamoeba histolytica, Entamoeba coli, Entamoeba dispar, Entamoeba hartmanni, Entamoeba polecki, Wuchereria bancrofti, Giardia, and Leishmania.

Methods for determining whether a composition of the present inventions is capable of effectively delivering the bioactive agent and/or having the desired effect in a subject are known in the art and not described herein in detail. In one aspect, immune responses against an antigen can be determined by monitoring the level antigen-specific antibody before and after administration (e.g., systemic IgM, IgG (IgG1, IgG2a, et al.) or IgA) in blood samples or from mucosal sites. Cellular immune responses also can be monitored after administration by assessing T and B cell function after antigen stimulation.

Another way of assessing the immunogenicity of the compositions or vaccines disclosed herein where the nucleic acid molecule (e.g., the RNA) encodes a protein antigen is to express the recombinant protein antigen for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

The efficacy of the compositions can also be determined in vivo by challenging appropriate animal models of the pathogen of interest infection.

In the implementations provided herein, the subject is a vertebrate (e.g., an animal including farm animals (cows, pigs, goats, chickens, horses, etc.), pets (cats, dogs, birds, etc.), and rodents (rats, mice, etc.), or a human). In one implementation, the subject is a human. In another implementation, the subject is a non-human mammal. In another implementation, the non-human mammal is a dog, cow, or horse.

### IX. Methods of Delivering a Bioactive Agent to a Cell

Provided herein are methods of delivering a bioactive agent to a cell, including the step of contacting the cell with a composition described herein. In some implementations, the bioactive agent is a nucleic acid. In some implementations, contacting the cell with the composition includes a step of administering the composition to a subject where the cell is in the subject. Such methods are useful in the delivery of antigen or antigen-encoding nucleic acids for generation of an immune response. Such methods are also useful for the delivery of antibody-encoding nucleic acids, protein or small molecule drugs, hormones, non-coding RNA molecules, and other bioactive agents for treatment of disease and health conditions.

The methods described herein for delivering a bioactive agent to a cell may find use in the treatment of diseases and health conditions including, without limitation, cancer, such as meningiomas, hepatic cell carcinoma, pancreatic tumors; allergy; infectious diseases including fungal, bacterial, or parasitic diseases; inflammatory diseases including psoriasis and arthritis and atrial-ventricular malformations; autoimmune diseases; and neurological diseases.

In implementations of methods of delivering a composition to a cell including the step of administering the composition to a subject where the cell is in the subject, typical routes of administration of the therapeutically effective amount of the composition include, without limitation, oral, topical, parenteral, sublingual, buccal, rectal, vaginal, intravenous, intradermal, transdermal, intranasal, intramucosal, or subcutaneous. In implementations, administration of the composition is intramuscular, parenteral, or intradermal. In such implementations, the subject is a vertebrate (e.g., an animal including farm animals (cows, pigs, goats, chickens, horses, etc.), pets (cats, dogs, birds, etc.), and rodents (rats, mice, etc.), or a human). In one implementation, the subject is a human. In another implementation, the subject is a non-human mammal. In another implementation, the non-human mammal is a dog, cow, or horse.

In an implementation the mode of delivery is intradermal. The intradermal delivery can be conducted by the use of microneedles, with height of less than 1 mm or 1000 micron; and more preferably with height of 500-750 micron. A microneedle injection device preferably has multiple needles, typically 3 microneedles.

One suitable microneedle injection device is The MicronJet600®. The MicronJet600® is a small plastic device equipped with 3 microneedles, each 600 micrometers (0.6 mm) in length. This device can be mounted on any standard syringe instead of a standard needle. The microneedles themselves are made of silicon crystal and are integrated (bonded) after cutting into rows to their polycarbonate base using biocompatible UV cured glue.

The microneedle injection device is facing “downward” (bevel down) i.e., the injection aperture is facing deeper into the skin, and not bevel up. This enables reliable injection without leakage. The injection orientation is preferably defined by visible or mechanical features of the base/adapter.

The microneedle injection is done into the shallow dermis, and the epidermis. This allows for effective expression and immunization. The injection depth with a microneedle is typically about 100-750 micron, and more preferably about 300-400 micron; This is in contrast with regular needles, or other mini or microneedles which typically deliver to a deeper layer of the skin or below the skin. The injection angle is preferably about 45 degrees (typically ±20°, and more preferably ±10°), allowing shallow injection point, relative to standard needles, and other perpendicular microneedles.

Provided herein is a system and method of delivering RNA including saRNA (self-amplifying RNA) into an animal or a human patient (e.g., a subject), comprising administering the RNA (e.g., saRNA) to the epidermis or the dermis of the skin at a depth of between about 100 and about 700 microns from the surface of the skin. An effective amount of RNA will be delivered to allow for expression of a protein encoded by the RNA. The protein can be an antigen as described herein and can be, for example, a vaccine component.

The RNA can be administered with an intradermal delivery device comprising one or more microneedles; wherein the intradermal delivery device is designed for shallow intradermal delivery. The RNA can be administered with an intradermal delivery device according to the teachings of U.S. Pat. No. 6,533,949 and/or U.S. Pat. No. 7,998,119.

Any of the RNA containing formulations and/or compositions described herein can be administered intradermally via a microneedle device as described herein. Other intradermal devices for delivery RNA can be used as well, including, for example, intradermal electroporation delivery devices. In some implementations, delivery of the RNA will generate an immune response in a subject.

In some implementations, multiple modes of delivery may be used to obtain greater immune response. For example, the composition can be administered 1, 2, 3, 4, 5, 6, or more times. In some implementation, the one or more administrations may occur as part of a so-called “prime-boost” protocol. In some implementations the “prime-boost” approach comprises administration in in several stages that present the same antigen through different vectors or multiple doses. In some implementations, administration may occur more than twice, e.g., three times, four times, etc., so that the first priming administration is followed by more than one boosting administration. When multiple vectors or doses are administered, they can be separated from one another by, for example, one week, two weeks, three weeks, one month, six weeks, two months, three months, six months, one year, or longer. In some implementations, a prime-boost approach comprises an RNA stage and a protein stage. The RNA stage may include, for example, administration of RNA carrying a gene coding for the antigenic protein, translation of the RNA into the antigen, and production of the corresponding antibodies in the subject. The protein stage may include, for example, administration of the antigen directly in the form of a protein. In some implementations, the subject is administered (e.g., primed with) an oncolytic virus (which may be formulated with an NLC or without an NLC) that encodes a neoantigen, and then subsequently administered (e.g., boosted with) an NLC comprising an RNA construct that encodes the neoantigen.

### XI. Kits and Articles of Manufacture

Also contemplated in certain implementations are kits comprising the herein described lyophilized nanostructured lipid carriers (NLCs) and compositions, which may be provided in one or more containers. In one implementation, all components of the compositions are present together in a single container. In other implementations, components of the compositions may be in two or more containers.

In some implementations, one vial of the kit comprises a lyophilized NLC provided herein, and a second vial of the kit contains a bioactive agent such as an RNA molecule. In some implementations, the kit comprises a third vial containing an additional or optional component.

The kits of the invention may further comprise instructions for use as herein described or instructions for mixing the materials contained in the vials. In some implementations, the material in the vial is dry or lyophilized. In some implementations, the material in one or more of the vials is liquid.

A container according to such kit implementations may be any suitable container, vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Typically, such a container may be made of a material that is compatible with the intended use and from which recovery of the contained contents can be readily achieved. Non-limiting examples of such containers include glass and/or plastic sealed or re-sealable tubes and ampules, including those having a rubber septum or other sealing means that is compatible with withdrawal of the contents using a needle and syringe. Such containers may, for instance, by made of glass or a chemically compatible plastic or resin, which may be made of, or may be coated with, a material that permits efficient recovery of material from the container and/or protects the material from, e.g., degradative conditions such as ultraviolet light or temperature extremes, or from the introduction of unwanted contaminants including microbial contaminants. The containers are preferably sterile or sterilizable, and made of materials that will be compatible with any carrier, excipient, solvent, vehicle or the like, such as may be used to suspend or dissolve the herein described vaccine compositions and/or immunological adjuvant compositions and/or antigens and/or recombinant expression constructs, etc.

### XII. Illustrative Implementations

Implementation 1. A thermostable, lyophilized composition for delivery of a bioactive agent to a cell, the composition comprising: a) nanostructured lipid carrier (NLC) particles comprising: an oil core comprising a mixture of a liquid phase lipid and a solid phase lipid; a cationic lipid; a hydrophobic surfactant; and a hydrophilic surfactant; and b) a cake-forming excipient, wherein the composition is in the form of a cake and forms an oil-in-water emulsion upon reconstitution.

Implementation 2. The composition of implementation 1, further comprising: c) the bioactive agent, wherein the bioactive agent comprises RNA.

Implementation 3. The composition of implementation 2, wherein the RNA comprises a replicon.

Implementation 4. The composition of implementation 2, wherein the RNA is self-amplifying RNA (saRNA).

Implementation 5. The composition of implementation 2, wherein the RNA is messenger RNA (mRNA).

Implementation 6. The composition of any of implementations 2-5, wherein the RNA encodes an antigen.

Implementation 7. The composition of implementation 6, wherein the antigen comprises the Zika pre-membrane (PrM) and envelope (E) proteins.

Implementation 8. The composition of implementation 6, wherein the antigen comprises the SARS-CoV-2 spike protein.

Implementation 9. The composition of any of implementations 2-8, wherein the bioactive agent is electrostatically complexed to the outer surface of the NLC particles.

Implementation 10. The composition of any of implementations 1-9, wherein the liquid phase lipid is metabolizable.

Implementation 11. The composition of any of implementations 1-10, wherein the liquid phase lipid is a vegetable oil, animal oil, or synthetically prepared oil.

Implementation 12. The composition of any of implementations 1-10, wherein the liquid phase lipid is capric/caprylic triglyceride, vitamin E, lauroyl polyoxylglyceride, monoacylglycerol, soy lecithin, squalene, synthetic squalene, squalene, or a combination thereof.

Implementation 13. The composition of any of implementations 1-10, wherein the liquid phase lipid is a naturally occurring or synthetic terpenoid.

Implementation 14. The composition of any of implementations 1-10, wherein the liquid phase lipid is squalene or synthetic squalene.

Implementation 15. The composition of any of implementations 1-14, wherein the solid phase lipid is a glycerolipid.

Implementation 16. The composition of any of implementations 1-14, wherein the solid phase lipid is a microcrystalline triglyceride.

Implementation 17. The composition of implementation 16, wherein the microcrystalline triglyceride is trimyristin.

Implementation 18. The composition of any of implementations 1-17, wherein the cationic lipid is 1,2-dioleoyloxy-3-(trimethylammonio)propane (DOTAP), 3β-[N-(N′,N′-Dimethylaminoethane)-carbamoyl]Cholesterol (DC Cholesterol), dimethyldioctadecylammonium (DDA), 1,2-Dimyristoyl-3-TrimethylAmmoniumPropane (DMTAP), dipalmitoyl(C16:0)trimethyl ammonium propane (DPTAP), distearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), or a combination thereof.

Implementation 19. The composition of implementation 18, wherein the cationic lipid is 1,2-dioleoyloxy-3-(trimethylammonio)propane (DOTAP).

Implementation 20. The composition of any of implementations 1-19, wherein the hydrophobic surfactant is a sorbitan ester.

Implementation 21. The composition of implementation 20, wherein the sorbitan ester is a sorbitan monoester.

Implementation 22. The composition of implementation 21, wherein the sorbitan monoester is sorbitan monostearate.

Implementation 23. The composition of implementation 21, wherein the sorbitan monoester is sorbitan monooleate.

Implementation 24. The composition of implementation 20, wherein the sorbitan ester is a sorbitan triester.

Implementation 25. The composition of implementation 24, wherein the sorbitan triester is sorbitan trioleate or sorbitan tristearate.

Implementation 26. The composition of any of implementations 1-25, wherein the hydrophilic surfactant is a polysorbate.

Implementation 27. The composition of implementation 26, wherein the polysorbate is polysorbate 80.

Implementation 28. The composition of any of implementations 1-27, wherein the cake-forming excipient is a saccharide.

Implementation 29. The composition of implementation 28, wherein the saccharide is sucrose.

Implementation 30. The composition of implementation 28, wherein the saccharide is trehalose.

Implementation 31. The composition of any of implementations 28-30, wherein the saccharide is present at about 10-20% w/v.

Implementation 32. The composition of implementation 31, wherein the saccharide is present at about 20% w/v.

Implementation 33. The composition of any of implementations 1-32, wherein the liquid phase lipid is squalene or synthetic squalene, the solid phase lipid is trimyristin, the cationic lipid is DOTAP, the hydrophobic surfactant is sorbitan monostearate, the hydrophilic surfactant is polysorbate 80, and the cake-forming excipient is sucrose.

Implementation 34. The composition of any one of implementations 1 or 10-33, wherein the z-average diameter of the NLC particles is from about 40 nm to about 60 nm.

Implementation 35. The composition of any one of implementations 2-33, wherein the z-average diameter of the NLC particles and bioactive agent is from about 90 nm to about 150 nm.

Implementation 36. The composition of any one of implementations 2-35, having a loading capacity for RNA of at least about 100 ng/μL RNA.

Implementation 37. The composition of implementation 36, having a loading capacity for RNA of at least about 200 ng/μL RNA.

Implementation 38. The composition of any one of implementations 2-37, having a nitrogen:phosphate (N:P) ratio of about 15.

Implementation 39. The composition of any one of implementations 1-38, comprising from about 0.2% to about 40% w/v liquid phase lipid, from about 0.1% to about 10% w/v solid phase lipid, from about 0.2% to about 10% w/v cationic lipid, from about 0.25% to about 15% w/v hydrophobic surfactant, from about 0.2% to about 15% w/v hydrophilic surfactant, and from about 15% to 25% w/v cake-forming excipient.

Implementation 40. The composition of implementation 39, about 3.75% w/v liquid phase lipid, about 0.24% w/v solid phase lipid, about 3% w/v cationic lipid, about 3.7% w/v sorbitan ester, about 3.7% w/v hydrophilic surfactant, and about 20% w/v cake-forming excipient.

Implementation 41. The composition of any one of implementations 39-40, wherein the cake-forming excipient is sucrose.

Implementation 42. The composition of any one of implementations 39-40, wherein the cake-forming excipient is trehalose.

Implementation 43. The composition of any one of implementations 1-42, wherein a hydrophilic surfactant to cationic lipid molar ratio is about 0.2 to about 1.5.

Implementation 44. The composition of implementation 43, wherein the hydrophilic surfactant to cationic lipid molar ratio is about 0.5 to about 1.

Implementation 45. The composition of any one of implementations 1-44, wherein an oil to surfactant molar ratio is about 0.05 to about 12.

Implementation 46. The composition of implementation 45, wherein the oil to surfactant molar ratio is about 0.5 to about 1.

Implementation 47. The composition of any one of implementations 1-46, wherein the composition is thermostable at about 25° C. for at least 6 months.

Implementation 48. The composition of implementation 47, wherein the composition is thermostable at about 25° C. for at least 8 months.

Implementation 49. The composition of any one of implementations 1-46, wherein the composition is thermostable at about 4° C. for at least 12 months.

Implementation 50. The composition of implementation 49, wherein the composition is thermostable at about 4° C. for at least 21 months.

Implementation 51. The composition of any one of implementations 47-50, wherein thermostability is determined by the cake maintaining size, structure, and color.

Implementation 52. The composition of any one of implementations 47-50, wherein thermostability is determined by assay of components of the oil-in-water emulsion following reconstitution.

Implementation 53. The composition of any one of implementations 47-50, wherein thermostability is determined by change in z-average diameter of less than 20%.

Implementation 54. The composition of any one of implementations 47-50, wherein thermostability is determined by RNA integrity.

Implementation 55. A method of generating a thermostable, lyophilized composition for delivery of a bioactive agent to a cell, the method comprising: generating NLC particles by mixing the solid phase lipid, the liquid phase lipid, the cationic lipid, and the hydrophobic surfactant to form an oil phase mixture; mixing the hydrophilic surfactant and an aqueous buffer to form an aqueous phase mixture; and mixing the oil phase mixture with the aqueous phase mixture; mixing the NLC particles with a buffer containing the cake-forming excipient; and lyophilizing the NLC particles with the buffer containing the cake-forming excipient wherein the composition is in the form of a cake and forms an oil-in-water emulsion upon reconstitution.

Implementation 56. The method of implementation 55, further comprising combining the NLC particles and buffer containing the cake-forming excipient with the bioactive agent such that the bioactive agent electrostatically complexes with the outer surface of the NLC particles.

Implementation 57. The method of implementation 56, wherein the bioactive agent is RNA and the NLC particles are combined with the bioactive agent at a nitrogen:phosphate (N/P) ratio of about 15.

Implementation 58. The method of any of implementations 55-57, wherein the cake-forming excipient is sucrose.

Implementation 59. The method of any of implementations 55-57, wherein the cake-forming excipient is trehalose.

Implementation 60. The method of any of implementations 58-59, wherein the composition prior to lyophilization comprises about 10-20% w/v of the cake-forming excipient.

Implementation 61. The method of implementation 60, wherein the composition prior to lyophilization comprises about 20% w/v sucrose.

Implementation 62. A method of stimulating an immune response in a subject comprising: reconstituting the cake of any one of implementations 1-54 into an oil-in-water emulsion; combining the oil-in-water emulsion with a bioactive agent; and administering to the subject in an amount effective to stimulate the immune response in the subject.

Implementation 63. A method of stimulating an immune response in a subject comprising: reconstituting the cake of any one of implementations 2-54 into an oil-in-water emulsion; and administering the emulsion to the subject in an amount effective to stimulate the immune response in the subject.

Implementation 64. The method of implementation 62 or 63, wherein the immune response is an antigen-specific immune response.

Implementation 65. The method of implementation 64, wherein the bioactive agent is RNA encoding the Zika pre-membrane (PrM) and envelope (E) proteins.

Implementation 66. The method of implementation 64, wherein the bioactive agent is RNA encoding the SARS-CoV-2 spike protein.

Implementation 67. The method of any of implementations 62-66, wherein the subject is a mammal.

Implementation 68. The method of any of implementations 62-66, wherein the oil-in-water emulsion is administered intramuscularly.

Implementation 69. The method of any of implementations 62-66, wherein the oil-in-water emulsion is administered intranasally.

### EXAMPLES

The following Examples are offered by way of illustration and not by way of limitation.

### Example 1: Stability of Liquid NLC Formulations

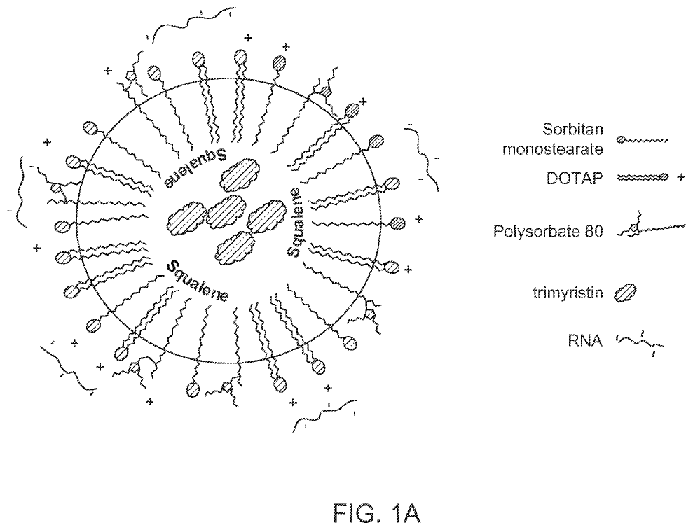

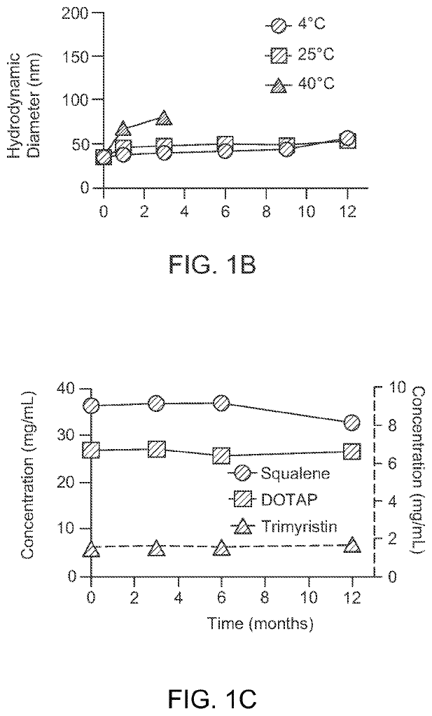

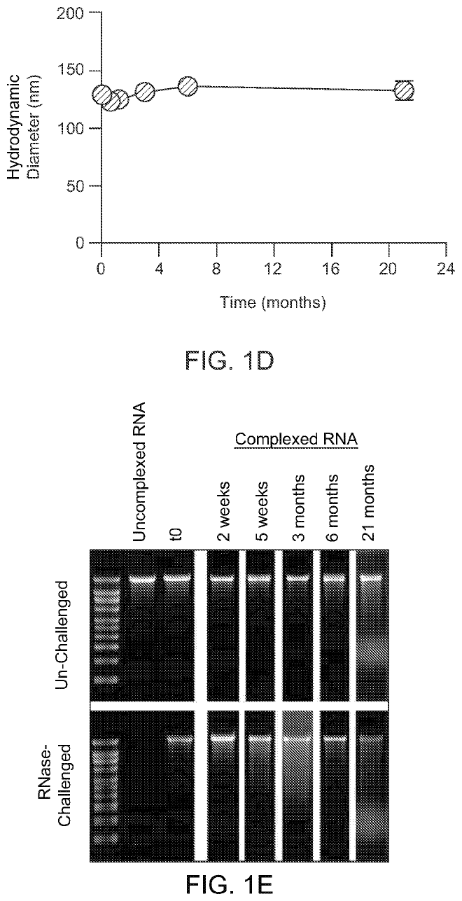

The NLC system itself displays long-term stability at 4° C., maintaining substantially the same particle size and component concentrations (FIGS. 1B and 1C), as well as retaining its ability to complex with and protect RNA from RNase challenge (FIG. 1E). Due to this long-term stability, uncomplexed NLC formulations are suitable for stockpiling as vaccine base formulations in advance. A bioactive agent targeting a specific pathogen can be produced as needed and complexed with pre-manufactured and stockpiled NLC formulations.

NLC Formulation

NLCs are composed of a hydrophobic core containing a liquid oil and a solid lipid, and surfactants (also known as emulsifiers or emulsifying agents) that make up the interface separating the hydrophobic phase—liquid oil and solid lipid, collectively referred to here as oil—from the aqueous phase. NLC compositions used in the examples consists of an oil core comprising a solid lipid (e.g., trimyristin or Dynasan®114) and a liquid lipid (e.g., squalene or synthetic squalene) surrounded by a hydrophilic surfactant (e.g., sorbitan monostearate or Span® 60), a hydrophilic surfactant (e.g., polysorbate 80 or TWEEN® 80) and a cationic lipid (e.g., DOTAP (N-[1-(2,3-Dioleoyloxy)propyl]-N,N,N-trimethylammonium chloride)). RNA, or other bioactive agent, which is negatively charged complexes electrostatically to the outside surface of the NLC particles as shown schematically in FIG. 1A.

The NLC formulation was prepared as described previously (J. H. Erasmus supra). Briefly, in order to synthesize NLC formulations, the oil phase was first prepared by mixing a liquid phase lipid squalene (Sigma), a solid phase lipid trimyristin Oleochemical), a positively charged lipid DOTAP (Corden), and a hydrophobic surfactant sorbitan monostearate (Sigma) in a blend vessel, which was placed in a sonicating water bath (60±5° C.) to facilitate solubilization. Separate preparation of the aqueous phase involved dilution of the hydrophilic surfactant polysorbate 80 (Fisher Scientific), in an aqueous buffer of 10 mM sodium citrate, followed by stirring for complete dissolution. The aqueous composition was also heated (60±5° C.) in a bath sonicator before blending with the oil phase.

After all components were dissolved, a high-speed laboratory emulsifier (Silverson Machines) was used to combine the oil and aqueous phases by blending at 7,000 RPM for a period of ten minutes to one hour to produce a crude mixture containing micron-sized oil droplets. The positioning of the Silverson mixing probe was adjusted as necessary for uniform dispersal of oil and complete emulsification. Further particle size reduction was achieved by high-shear homogenization in a M-110P microfluidizer (Microfluidics, Corp.). Each emulsion was processed for approximately 10 passes on the microfluidizer at 30,000 psi. The final pH was between 6.5-6.8. The resulting NLC particle suspension was terminally filtered with a 0.22 μm polyethersulfone filter (e.g., syringe filter) in order to collect the final NLC formulation. The final NLC formulation was stored at 2-8° C. until use.

NLC/RNA complexes were prepared at a nitrogen:phosphate (N/P) ratio of 15 for all examples. The Nitrogen to Phosphate (N/P) ratio is a theoretical representation of the molar stoichiometry of cationic nitrogens (positive charge) and anionic phosphate groups (negative charge) available to form the RNA-NLC complex. The cationic lipid DOTAP used in the NLCs contains a quaternary trimethylammonium head group and carries a positive charge that is independent of pH. Because each DOTAP molecule contains one trimethylammonium head group, nitrogen concentration (or the amount of positive charge) is essentially equal to DOTAP molar concentration. On the other hand, each ribonucleotide monophosphate in a RNA copy has a negative charge from the phosphate group so the phosphate concentration (or amount of negative charge) is approximately proportional to the RNA molar concentration normalized to the average molecular weight of ribonucleotide monophosphates (approximately 320-340 g/mol). Thus,

$$N / P = \frac{[ DOTAP ]}{[ RNA ] / \sim 330} ,$$

where [DOTAP] and [RNA] are molar concentrations of DOTAP and RNA, respectively.

Fresh complexes were prepared by mixing RNA 1 with NLC plus the desired amounts of sodium citrate and sucrose to achieve a final complex containing 200 ng/μL RNA in 2-5 mM sodium citrate and either 10% or 20% w/v sucrose aqueous buffer. RNA was added to the NLC formulation and gently pipetted up and down to ensure complete mixing. Complexes were incubated on ice for 30 minutes after mixing to ensure complete complexing.

Particle Size Stability

FIG. 1B shows long-term stability of the NLC formulation alone without RNA after storing at 4° C., 25° C., or 40° C. The NLC formulation maintained substantially the same particle size for 12 months when stored at 4° or 25° C.

To assess change in particle size, the average hydrodynamic diameter (Z-average) was measured using Dynamic Light Scattering (DLS) (Zetasizer Nano ZS, Malvern Instruments) at multiple timepoints over 12 months. The NLC formulations were diluted 1:100 with nuclease-free water in triplicate preparations and measured in a disposable polystyrene cuvette (SOP parameters: material RI=1.59, dispersant RI (water)=1.33, T=25° C., viscosity (water)=0.887 centipoise (cP), measurement angle=173° backscatter, measurement position=4.65 mm, automatic attenuation).

FIG. 1D shows the particle size of NLC/RNA complexes formed using NLC that had been stored at 4° C. for the indicated length of time. The NLC formulation was stored at 4° C. and complexed with SEAP saRNA at each timepoint indicated. Particle size measured using DLS at each timepoint over 21 months. The NLC/RNA complexes were substantially the same particle size at each timepoint as those measured at t0. Thus, the NLC retained ability to complex with RNA after storage at 4° C.

NLC Formulation Component Assay

The concentrations of DOTAP, squalene, and trimyristin in the NLC were determined by High Performance Liquid Chromatography (HPLC) at various timepoints over one year in storage at 4° C. as shown in FIG. 1C. The concentration of squalene decreased slightly at 12 months. Other concentrations remained stable.

Samples were prepared in triplicate, diluted 1:20 in HPLC mobile phase B (50 μL sample into 950 μL mobile phase B), injected at 10 μL injection volume, then analyzed using an Agilent 1100 quaternary pump HPLC system in combination with a Corona Veo charged aerosol detector (CAD). The method utilized a Phenomenex Synergi Hydro RP C18 80 A column (4 μm 4.6×250 mm) with a two solvent system gradient consisting of a mixture of 75:15:10 methanol:chloroform:water (mobile phase A) and a 1:1 mixture of methanol:chloroform (mobile phase B), with both mobile phases containing 20 mM ammonium acetate and 1% acetic acid. The system was held at 35° C. and run at a flow rate of 1 mL/min. DOTAP, trimyristin, and squalene were dissolved in mobile phase B, and the injection volume was varied to create a 5-point standard curve.

Protection from RNase Challenge

FIG. 1E shows protection of SEAP saRNA from RNase challenge by complexing with the NLC formulations stored at 4° C. for the indicated time. The SEAP saRNA was generated as described in Example 7 below and complexed with the NLC formulation at each timepoint indicated. The intensity of the intact saRNA bands remained constant for the full 21 months for un-challenged samples. For the samples challenged with RNase, there was a modest decrease in band intensity at 21 months.

Integrity of RNA after complexing and protection against RNase challenge was evaluated by agarose gel electrophoresis. All samples (fresh, frozen/thawed, or lyophilized/reconstituted) were diluted to a final RNA concentration of 40 ng/μL in nuclease-free water. For RNase-challenged samples, the diluted RNA was incubated with RNase A (Thermo Scientific) for 30 minutes at room temperature at amounts sufficient to completely degrade uncomplexed RNA (ratio of 1:40 RNase:SEAP-RNA).

This was followed by treatment with recombinant Proteinase K (Thermo Scientific) at a ratio of 1:100 RNase A:Proteinase K for 10 minutes at 55° C. For both challenged and un-challenged samples, RNA was then extracted from the complexes by adding 25:24:1 phenol:chloroform:isoamyl alcohol (Invitrogen) to the complex 1:1 by volume, vortexing, and centrifuging at 17,000 g for 15 minutes. The supernatant for each sample was mixed 1:1 by volume with Glyoxal load dye (Invitrogen) and incubated at 50° C. for 20 minutes. For each complex, 200 ng of RNA was loaded and run on a denatured 1% agarose gel at 120 V for 45 minutes in Northern Max Gly running buffer (Invitrogen). Uncomplexed RNA was included in each gel as a control for the activity of RNase. Gels were imaged using ethidium bromide protocol on a ChemiDoc MP imaging system (BioRad).

### Example 2: Evaluation of Cake-Forming Excipient on Cake Formation and Reconstitution

The selection and amount of saccharide used as a cake-forming excipient affected the reconstitution of the cake following lyophilization. Compositions were prepared with RNA complexed to NLC, NLC alone, and RNA alone. SEAP-saRNA as described in Example 7 was used for both the NLC/RNA samples and the RNA only samples. The NLC formulation was prepared as described above in Example 1. RNA was complexed to the NLC at a 15:1 N/P ratio with a RNA concentration of 200 ng/μL. The RNA only samples contained 400 ng/μL of RNA. The NLC samples were diluted 2.5 fold.

The NLC/RNA complex was lyophilized using a Virtis AdVantage 2.0 EL-85 bench-top freeze dryer controlled by the microprocessor-based Wizard 2.0 software. The lyophilization cycle consisted of a freezing step at −50° C., a primary drying step at −30° C. and 50 mTorr, and a secondary drying step at 25° C. and 50 mTorr. At the completion of the cycle, samples were brought to atmospheric pressure, blanketed with high purity nitrogen, and stoppered prior to being removed from the freeze-dryer chamber. Lyophilized material was reconstituted using nuclease-free water and gently swirled.

The lyoprotectants sucrose and trehalose were both evaluated at concentrations of 10% and 20% w/v in the formulations prior to lyophilization. Samples containing water without a lyoprotectant were also tested. The NLC/RNA samples have the following compositions: RN0 water, RN1 10% sucrose, RN2 20% sucrose, RN3 10% trehalose, and RN4 20% trehalose. The NLC only samples have the following compositions: NO water, N1 10% sucrose, N2 20% sucrose, N3 10% trehalose, and N4 20% trehalose. The RNA only samples have the following compositions: R0 water, R1 10% sucrose, R2 20% sucrose, and R3 10% trehalose.

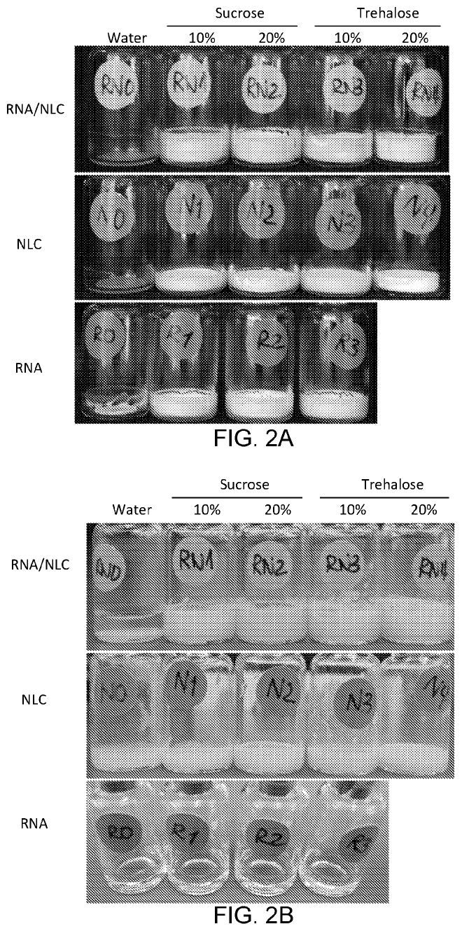

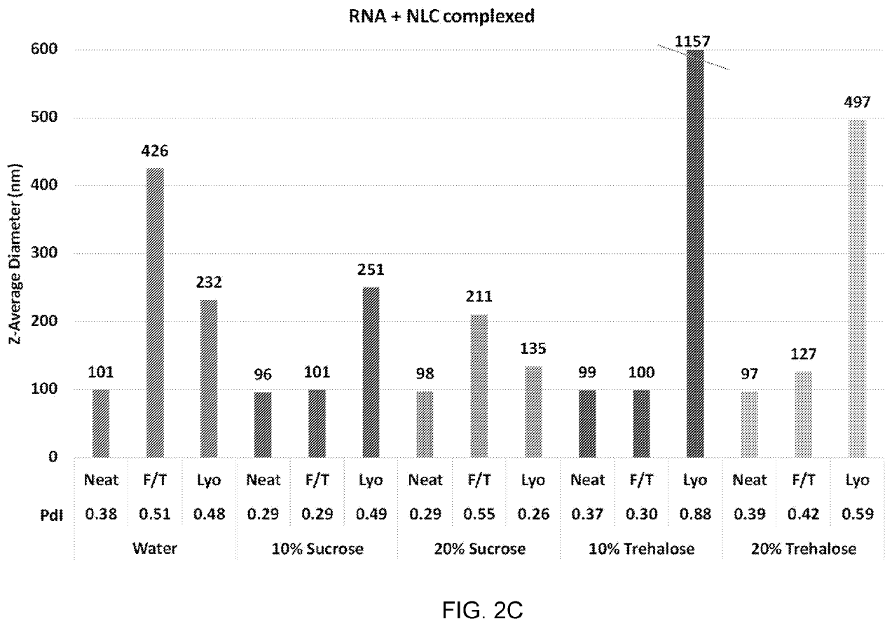

FIG. 2A shows vials containing lyophilized samples prior to reconstitution. FIG. 2B shows the reconstituted samples. The NLC/RNA samples with 10% saccharide took about 45-50 seconds to reconstitute while the samples with 20% saccharide took about 2.5 minutes to reconstitute. Sample RN0 prepared without a saccharide required rigorous vortexing and did not fully reconstitute. The samples with 10% sucrose and 10% or 20% trehalose were more opaque following reconstitution than before lyophilization and appear to contain very fine residual precipitates. Sample RN2 in 20% sucrose was only sample that returned to the original pre-lyophilization appearance.

The samples containing NLC only lyophilized with trehalose crashed out and eventually return to solution over approximately 30 minutes. After returning to solution the samples with trehalose were more opaque and viscous than the sucrose containing samples. The sample in 10% sucrose, N1, required 30 minutes to reconstitute. The sample in 20% sucrose, N2, reconstitute a milky white solution in 60 seconds.

All of the RNA samples, with and without lyoprotectants, reconstituted easily yielding clear colorless solutions with some bubbles which dissipated with time.

The identity of the lyoprotectant also affected particle size stability as shown in FIG. 2C. Particle size was measured by DLS as described above. SEAP-saRNA was complexed with NLC at a 15:1 N/P ratio and particle size was measured either of the freshly mixed sample (“neat”), after freezing at −80° C. followed by thawing to room temperature (“F/T”), or following lyophilization and reconstitution (“Lyo”). Particle size for all freshly prepared samples was around 100 nm. All lyophilized samples exhibited an increase in particle size. The increase was least for samples lyophilized in the presence of 20% sucrose followed by 10% sucrose, 20% trehalose, and 10% trehalose.

### Example 3: Evaluation of ZIKA saRNA Integrity and Protection after Lyophilization/Reconstitution

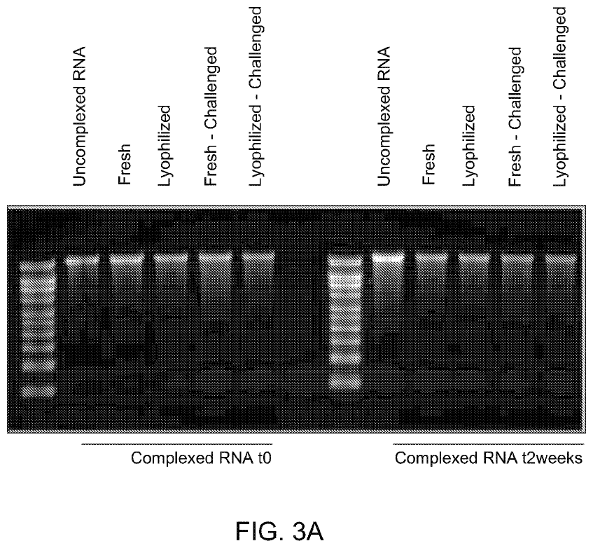

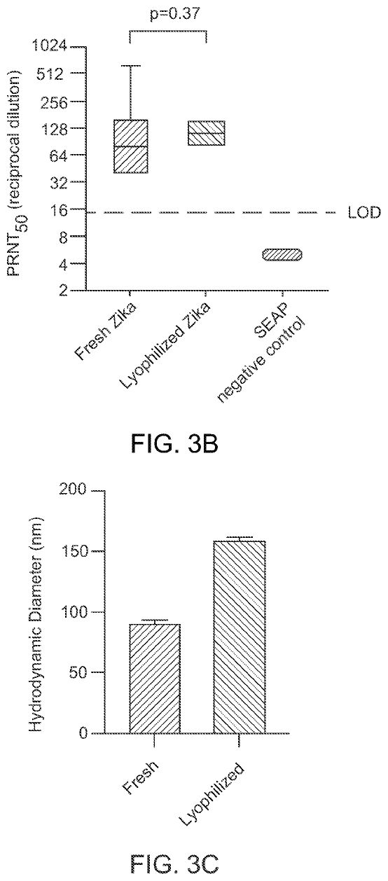

The effect of lyophilization and short-term 4° C. storage (2 weeks) on Zika saRNA complexed with NLC formulations was evaluated by agarose gel electrophoresis following RNase challenge (FIG. 3A), in vivo immunogenicity (FIG. 3B), and particle size (FIG. 3C). NLC formulations were prepared as described in Example 1 in 10 mM sodium citrate and then diluted 2.5 fold in 20% w/v sucrose. Zika saRNA prepared as described in Example 7 was mixed 1:1 by volume with the diluted NLC resulting in a final complex containing 200 ng/μL RNA in an isotonic 2 mM sodium citrate and 10% w/v sucrose aqueous buffer. Complexes were incubated on ice for 30 minutes after mixing to ensure complete complexing.

Samples were lyophilized as described above in Example 2. Reconstituted material following lyophilization was diluted to 5 mM sodium citrate and 10% w/v sucrose (for isotonicity) prior to in vivo experiments.

Protection from RNase Challenge

FIG. 3A shows the integrity of Zika saRNA in both freshly mixed and lyophilized/reconstituted vaccine after extraction from the NLC without challenge (“Unchallenged)” and after it has been challenged with RNase and then extracted from the NLC (“Challenged”). The NLC formulations retained their ability to protect from RNase challenge following lyophilization. RNA integrity was evaluated by forming the NLC/RNA complexes and then extracting the RNA immediately after lyophilization (t0) and after two weeks (t2 weeks) of storage at 4° C. The RNase challenge and running of the agarose gel were performed as described above in Example 1. RNase A was added at a ratio of 1:200 RNase:Zika-RNA, a ratio sufficient to completely degrade uncomplexed Zika-RNA.

Zika NLC/saRNA In Vivo Immunogenicity

Upon reconstitution and intramuscular injection into C57BL/6 mice, the lyophilized Zika saRNA vaccine is able to induce neutralizing antibody titers without significant difference from freshly-complexed, un-lyophilized vaccine at the same 1 μg dose. FIG. 3B shows in vivo immunogenicity equivalence of fresh and lyophilized/reconstituted Zika vaccine by PRNT. SEAP NLC/saRNA was used as an in vivo negative control that does not induce neutralizing antibodies to Zika. A sample size of 10 mice was used in each of the three groups. Comparability of PRNT titers between lyophilized and freshly complexed vaccine presentations for the saRNA Zika vaccine were conducted by a 2-tailed homoscedastic t-test on natural log-transformed PRNT titers. Log-transformed data were visually assessed for normality prior to analysis.

C57BL/6J mice between 4 and 8 weeks of age at study onset obtained from The Jackson Laboratory were used for all animal studies in these examples. All animal work was done under the oversight of IDRI's Institutional Animal Care and Use Committee and/or the Bloodworks Northwest Research Institute's Institutional Animal Care and Use Committee and is in compliance with all applicable sections of the Final Rules of the Animal Welfare Act regulations (9 CFR Parts 1, 2, and 3). Mice were non-specifically and blindly distributed into their respective groups. No exclusion criteria were established prior to beginning the studies.

To compare immunogenicity of lyophilized/reconstituted versus freshly complexed Zika NLC/saRNA vaccines, mice (n=10/group) were immunized with 1 μg of freshly complexed Zika NLC/saRNA vaccine, 1 μg lyophilized/reconstituted Zika NLC/saRNA vaccine, or 10 μg of SEAP NLC/saRNA complex as a negative control. The complexes were injected intramuscularly in 50 μl volumes in both rear quadriceps muscles of each mouse for a total of 100 μl vaccine per mouse. Injections sites were monitored for signs of reactogenicity for the 3 days post-injection, with no such signs noted. Blood samples were taken from all immunized mice 14 days post-immunization by the retro-orbital route for serum antibody assays by PRNT. Serum was harvested following low-speed centrifugation and stored at −20° C. until assayed.

Fifty percent plaque-reduction neutralization tests (PRNT₅₀ assays) were performed on mouse serum samples to quantify neutralizing antibody titers (Sornjai, Wannapa et al. “Analysis of Zika virus neutralizing antibodies in normal healthy Thais.” Scientific reports vol. 8, 1 17193. 21 Nov. 2018). Vero (ATCC CCL-81) cells were cultured at standard conditions (37° C., 5% CO₂) in antibiotic-free high-glucose DMEM supplemented with GlutaMax (Gibco) and 10% v/v heat-inactivated FBS (HyClone). Cells were plated at a density of 5×10⁵ cells/well in 6 well plates (Corning) and incubated overnight to form 90% confluent monolayers. Mouse serum samples were serially diluted 1:2 in DMEM containing 1% heat-inactivated FBS. All serum dilutions were then diluted 1:2 with 100 PFU of ZIKV strain FSS13025 and incubated at 37° C. for 1 hr. Cell supernates were removed and replaced with 200 μl of the virus/serum dilutions and allowed to incubate at culture conditions for 1 hour with gentle rocking every 20 minutes. Two ml of overlay medium comprised of DMEM containing 1% agarose (SeaKem), GlutaMax, and 1% v/v FBS was added to each well, allowed to solidify, and plates were incubated for 3 days at standard culture conditions. Cells were then fixed in 10% formalin (Fisher Scientific) for 20 minutes and stained with crystal violet for plaque visualization and counting.

Particle Size Stability

FIG. 3C shows hydrodynamic diameter of fresh and lyophilized/reconstituted vaccine measured by DLS as described above. The size of the complex has a moderate increase post-lyophilization and reconstitution from about 90 nm to about 150 nm which does not appear to affect in vivo efficacy as shown by the PRNT assay illustrated in FIG. 3B.

### Example 4: Evaluation of OVA mRNA Integrity and Particle Size after Lyophilization/Reconstitution

Commercially-available mRNA encoding ovalbumin (OVA) (TriLink CleanCap OVA mRNA, L-7610) was complexed with the NLC compositions of Example 1 with 20% w/v sucrose added during complexing. The NLC-based system of this disclosure protects mRNA equally well as saRNA indicating that protection does not depend on the size and type of RNA. Lyophilized or frozen OVA NLC/mRNA was compared with freshly complexed material to evaluate protection from RNase challenge and change in particle size.

Protection from RNase Challenge

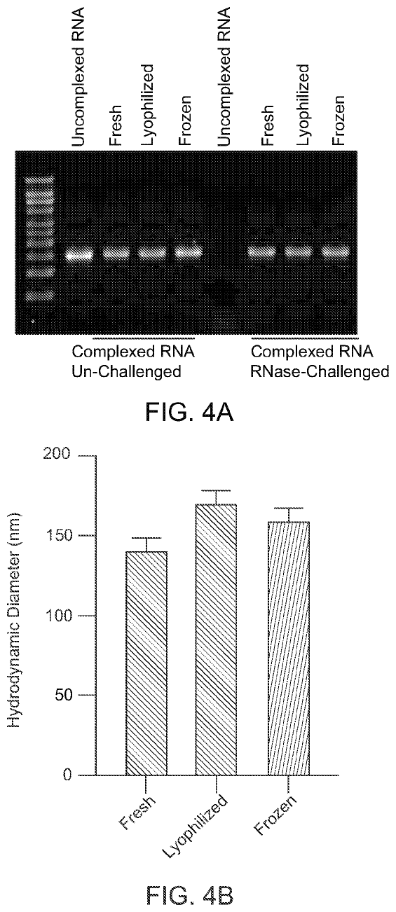

FIG. 4A shows integrity of OVA mRNA under fresh, frozen (−80° C. three days), or lyophilized conditions after it has been extracted from the NLC complex (“Un-Challenged”) and protection of OVA mRNA after it has been challenged with RNase and then extracted from the NLC complex (“Challenged”). The RNase challenge and running of the agarose gel were performed as described above in Example 1. RNase A was added at a ratio of 1:40 RNase:OVA mRNA, a ratio sufficient to completely degrade uncomplexed OVA mRNA. Complexing with the NLC formulation protected the mRNA from RNase challenge across all tested storage conditions.

Particle Size Stability

FIG. 4B shows hydrodynamic diameter of fresh, frozen, and lyophilized/reconstituted complexes measured by DLS as described above. The average particle size (n=3) of the lyophilized/reconstituted complexes is about 170 nm which is slightly higher than the 150 nm hydrodynamic diameter measurement for the lyophilized/reconstituted Zika NLC/saRNA vaccine (FIG. 3C). This difference in size is believed to be due to increasing the concentration of the lyoprotectant sucrose to 20% w/v, the particle size exhibits only a slight increase post-lyophilization and reconstitution and is consistent with the size after freezing and thawing.

### Example 5: Evaluation of Long-Term Stability of Lyophilized SEAP saRNA and NLC Complexes

The long-term thermostability of the NLC-based RNA vaccine platform using a self-amplifying RNA antigen expression reporter system expressing secreted alkaline phosphatase (SEAP-saRNA) is demonstrated through serum detection of the reporter. The SEAP-saRNA was created as described below in Example 7. The NLC formulation was created as described in Example 1 with 20% sucrose added during complexing. The NLC and RNA were mixed to achieve a final complex containing 200 ng/μL RNA in an isotonic 2 mM sodium citrate and 20% w/v sucrose aqueous buffer. Complexes were incubated on ice for 30 minutes after mixing to ensure complete complexing. The NLC/RNA complex was lyophilized as described in Example 2. Lyophilized SEAP-saRNA complexes with 20% w/v sucrose as a lyoprotectant stored at 4° C., 25° C., and 40° C. were compared with frozen complexes stored at −80° C. and −20 C°, liquid complexes stored at 4° C. and 25° C., and freshly made complexes prepared each analysis day.

Cake Structure and Reconstitution

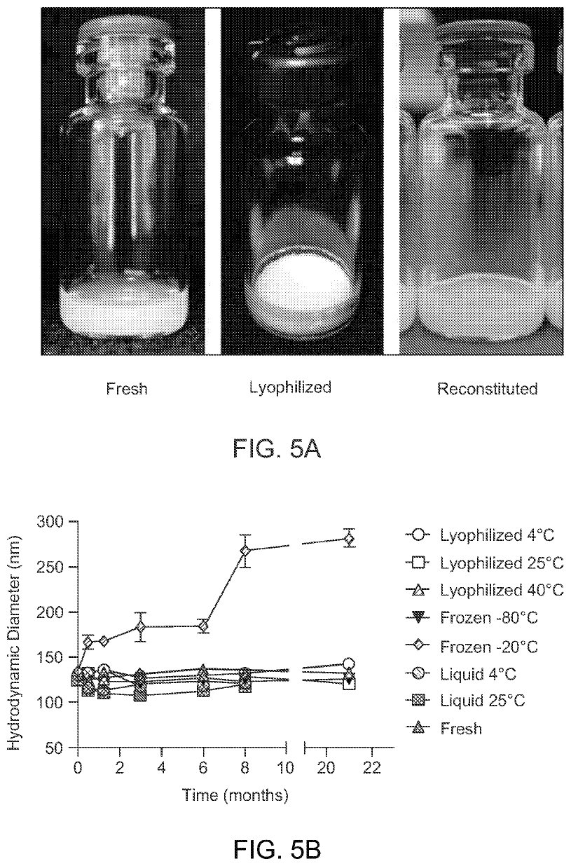

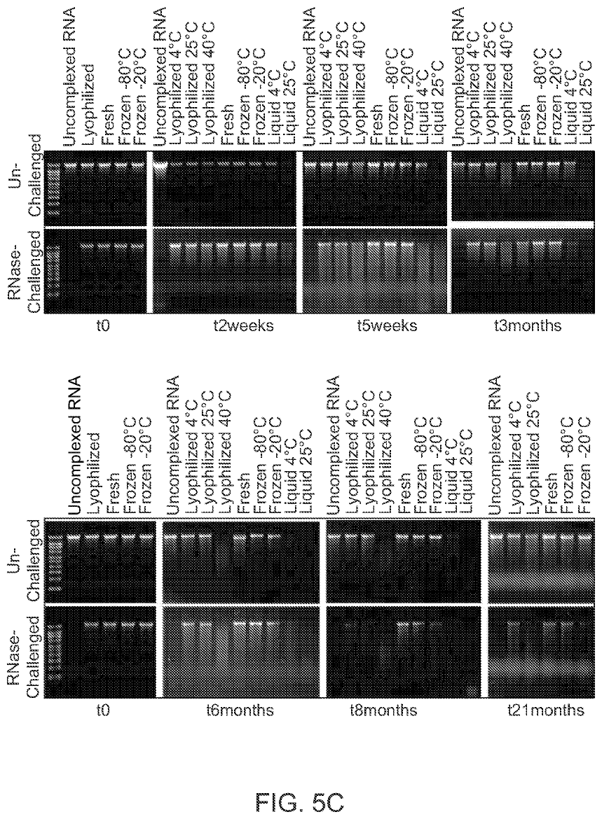

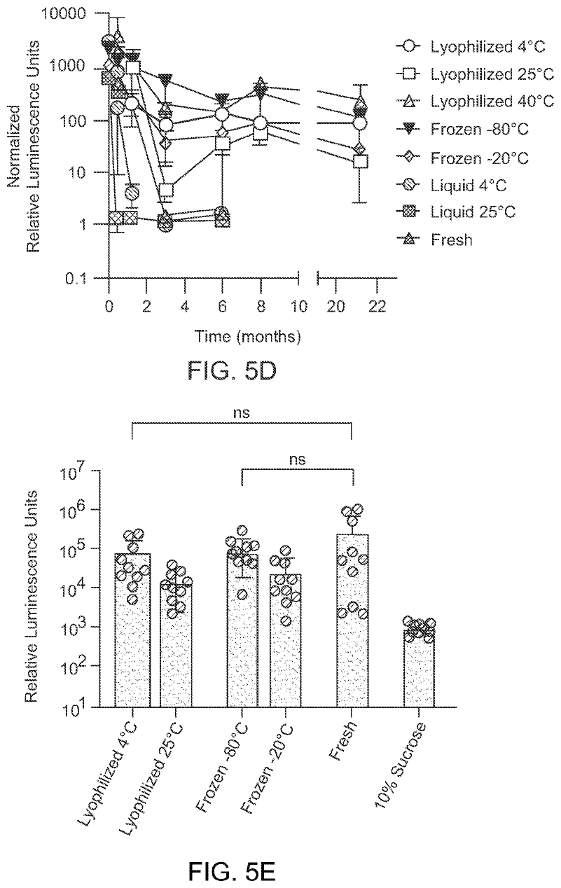

FIG. 5A shows vial images of freshly complexed, lyophilized, and reconstituted material at t0. All lyophilized samples maintained an elegant, white cake throughout the study with no discoloration or cracking and minimal cake shrinkage. All lyophilized samples readily reconstituted with nuclease-free water, and the reconstituted complexes were visually similar to freshly-complexed comparators. Lyophilized and reconstituted complexes of NLC/Zika saRNA (Example 2) and NLC/OVA mRNA (Example 3) exhibited similar cake structure and reconstituted appearance (vial images not shown). Thus, indicating the cake structure and successful reconstitution is not dependent on the RNA.

Particle Size Stability

FIG. 5B shows hydrodynamic diameter of the complexes over time as compared to a freshly complexed control. Initially, all NLC/saRNA complexes measured 125±10 nm in diameter, including liquid, frozen, and lyophilized versions. Differences of less than 15% in particle size were observed between the initial and final timepoints for all conditions except frozen material stored at −20° C. The lyophilized samples stored at 4° C. and 25° C. maintained substantially the same particle size for at least 21 months. This demonstrates the excellent colloidal stability of NLC/RNA complexes, allowing them to withstand the stresses of the lyophilization process and long-term storage (i.e., at least 8 months), even at elevated temperatures (40° C. for lyophilized storage and 25° C. for liquid storage). It is interesting to note that, while size stability was not maintained for complexes stored at −20° C., this did not impact the ability of the NLC/saRNA complex to express protein in vivo as shown in FIGS. 5D and 5E.

Protection from RNase Challenge

FIG. 5C shows RNA integrity of the stored samples and protection from RNase challenge at multiple timepoints from t0 to 21 months. The lyophilized samples maintained RNA integrity and protection against RNase challenge for at least 21 months when stored at refrigerated (4° C.) temperatures. Under accelerated conditions, degradation in the form of reduced protection from RNase challenge was observed at 2 weeks for the liquid 25° C. condition, at 5 weeks for the liquid 4° C. condition, and at 3 months for the lyophilized 40° C. condition.

The RNase challenge and running of the agarose gel were performed as described above in Example 1. RNase A was added at a ratio of 1:40 RNase:SEAP-RNA, a ratio sufficient to completely degrade uncomplexed SEAP-RNA

In Vivo Functionality of Stored SEAP NLC/saRNA

FIG. 5D shows normalized in vivo SEAP expression for lyophilized, frozen, or liquid stored samples at various temperatures in comparison with freshly complexed material after long-term storage. RNA integrity in the NLC/saRNA complexes was maintained after lyophilization and after freeze/thaw. Lyophilized complex stored at 4° C. (open circle) maintained in vivo expression ability for at least 21 months. After 8 months of storage, lyophilized complex stored at 4° C. (open circle) and 25° C. (open square) and complex stored frozen at −80° C. (solid-filled triangle) and −20° C. (diamond) showed comparable levels of mouse serum SEAP expression to the freshly complexed material (shaded triangle).

FIG. 5E shows, surprisingly, no significant difference (p>0.05) in in vivo SEAP expression at 21 months for lyophilized vaccine stored at 4° C., frozen vaccine stored at −80° C., and freshly-prepared vaccine; 10% sucrose group shown as control. Comparability of SEAP expression levels at 21 months for each stored sample to a freshly complexed control was conducted using Dunnett's multiple comparisons test on the data prior to normalization. This demonstrates that RNA complexed with NLC and lyophilized may be stored long-term at refrigerated temperatures without a deep cold chain.

C57BL/6 mice (n=5 for t0 to t8 months and n=10 for t21 months) received a total dose of 100 ng RNA in a single 50 μL i.m. injection in one hind leg. A control group of mice received a 50 μL i.m. injection of 10% sucrose in a hind leg. Blood samples were taken from all immunized mice on day 5 post-injection by the retro-orbital route and serum was harvested following low-speed centrifugation and stored at −20° C. until assayed.

Serum samples were assayed for SEAP expression using the NovaBright Phospha-Light EXP Assay Kit for SEAP (ThermoFisher) according to the manufacturer's directions. Relative luminescence was measured using a Biotek Synergy2 plate reader. At each timepoint, SEAP expression for sample at each storage condition was normalized in FIG. 5D to the SEAP expression of the 10% sucrose control with 1 luminescence unit corresponding to the expression of the control.

### Example 6: Stability of Lyophilized SARS-CoV-2 RNA/NLC Vaccine

The thermostability of the NLC-based RNA vaccine platform using a self-amplifying RNA antigen expressing SARS-CoV-2 Spike protein is evaluated to determine if immunization elicited an antibody-specific response after storage of the lyophilized and frozen vaccine. Self-amplifying SARS-CoV-2 RNA was created from DNA templates as described below in Example 7. The NLC formulation was created as described in Example 1. The NLC and RNA were mixed to achieve a final complex containing 200 ng/μL RNA in an isotonic 2 mM sodium citrate and 20% w/v sucrose aqueous buffer. Complexes were incubated on ice for 30 minutes after mixing to ensure complete complexing. The NLC/RNA complex was lyophilized as described in Example 2.

To compare the effect of various storage conditions on the immunogenicity of SARS-CoV-2 NLC/saRNA vaccines, mice (n=5/group) were immunized i.m. with 10 μg of complexed SARS-CoV-2 NLC/saRNA vaccine either freshly prepared, stored for one month either frozen at −80° C., lyophilized and stored at 4° C., lyophilized and stored at 25° C., or lyophilized and stored at 40° C. Serum was collected 14 days following inoculation and SARS-CoV-2 specific IgG in the serum was determined by ELISA using recombinant SARS-CoV-2 spike protein-coated microtiter plates for SARS-CoV-2 spike protein-binding antibody capture, dilutions of a monoclonal SARS-CoV-2 IgG antibody as an assay standard, and a alkaline phosphatase-conjugated secondary anti-mouse total IgG antibody for detection.

Protection from RNase Challenge

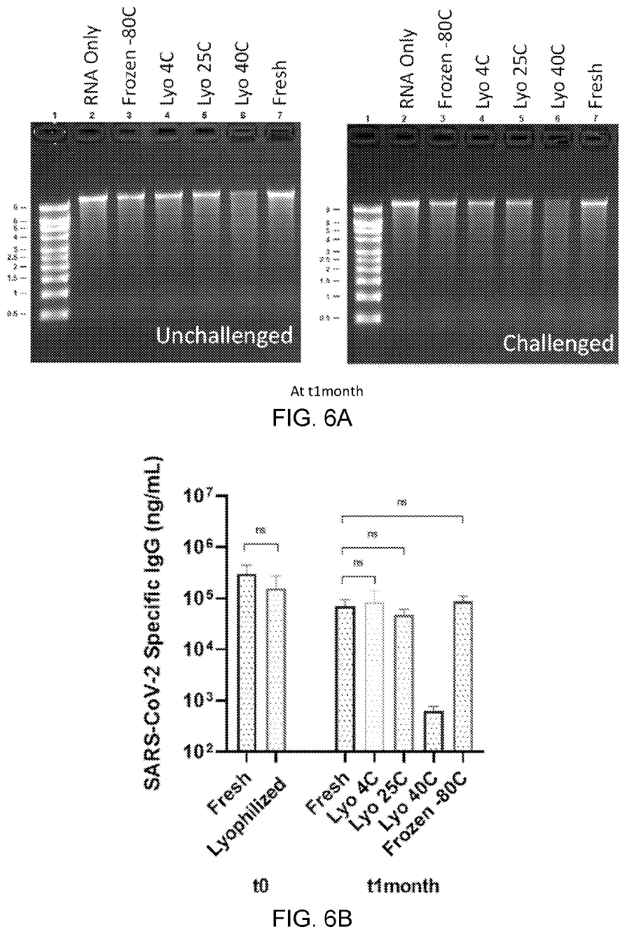

FIG. 6A shows RNA integrity in freshly mixed, frozen, and lyophilized/reconstituted vaccine after extraction from the NLC without challenge (“Unchallenged”) and after it has been challenged with RNase and then extracted from the NLC (“Challenged”). The sample containing RNA only was not challenged in either gel. The NLC formulations retained their ability to protect from RNase challenge following lyophilization. RNA integrity was evaluated by forming the NLC/RNA complexes and then extracting the RNA immediately after lyophilization (t0) and after one month (t1month) of storage at the indicated temperatures. The sample stored at 40° C. degraded. The RNase challenge and running of the agarose gel were performed as described above in Example 1. RNase A was added at a ratio of 1:500 RNase:SARS-CoV-2-RNA, a ratio sufficient to completely degrade uncomplexed SARS-CoV-2-RNA.

SARS-CoV-2 NLC/saRNA In Vivo Immunogenicity

Upon reconstitution and intramuscular injection into C57BL/6 mice, the lyophilized SARS-CoV-2 saRNA vaccine is able to induce specific antibody responses indicating this is a thermostable platform for a SARS-CoV-2 vaccine. Serum from immunized mice was titrated to find endpoint titer (last optical density (OD) value greater than a threshold determined by sera from unimmunized mice). The complexes were injected intramuscularly in 50 μl volumes in both rear quadriceps muscles of each mouse for a total of 100 μl vaccine per mouse, equivalent to a 10 μg total dose of saRNA. Injections sites were monitored for signs of reactogenicity for the 3 days post-injection, with no such signs noted. Blood samples were taken from all immunized mice 14 days post-immunization by the retro-orbital route for serum antibody assays by ELISA. Serum was harvested following low-speed centrifugation and stored at −20° C. until assayed.

At time zero, there was no significant difference in immunogenicity between freshly prepared samples and lyophilized samples showing that lyophilization does not affect the immunogenicity of the saRNA/NLC. After one month of storage, there was no significant difference in the IgG titer between the freshly prepared sample and frozen sample stored at −80° C. or the lyophilized samples stored at 4° C. or 25° C. There was a decrease in the antibody response for the lyophilized sample stored at 40° C. The difference in immunogenicity between the samples analyzed at time zero and at one month is likely due to day-to-day variations in the assay as the fresh control at one month is lower than the fresh control at time zero. Data are shown with height of the bars as the mean and error bars indicating standard deviation (n=10). Significance was identified by a 2-tailed homoscedastic t-test on log-transformed data.

### Example 7: Production of saRNA

saRNA DNA Templates

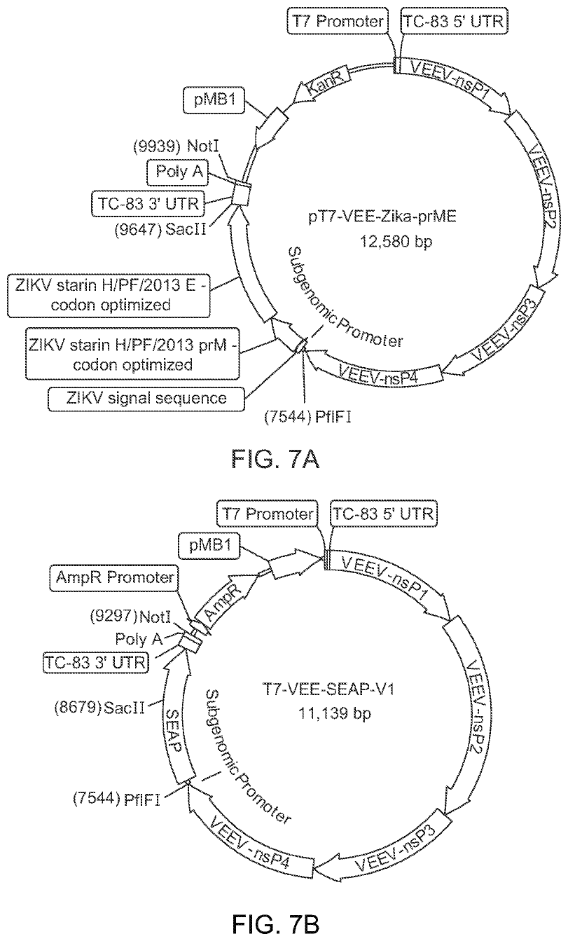

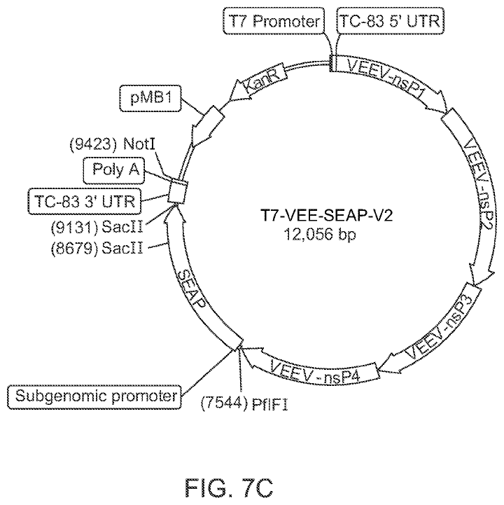

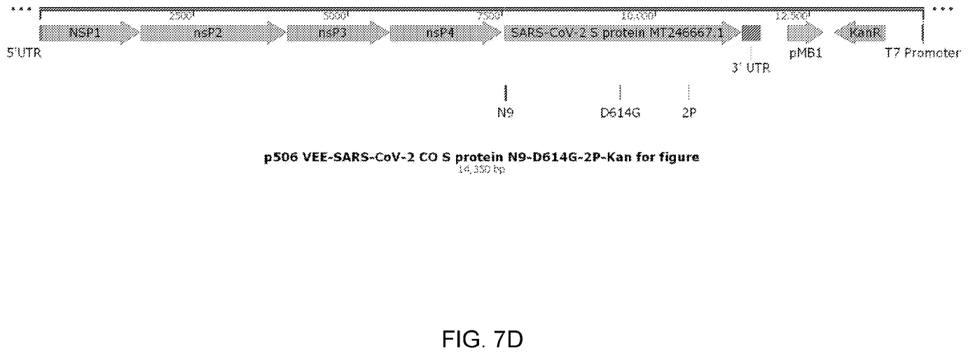

DNA templates for self-amplifying RNA (saRNA) encoding the Zika pre-membrane (PrM) and envelope (E) proteins were produced as previously described (J. H. Erasmus supra). Briefly, sequences for the Zika virus signal peptide at the N-terminal end of the capsid protein through the prM and E genes were taken from ZIKV strain H/PF/2013 (GenBank Accession #KJ776791), codon-optimized for mammalian expression, and subcloned into a T7-TC83 plasmid. The codon-optimized ZIKV prM and E genes are SEQ ID NO: 1. The resulting plasmid pT7-VEE-Zika-prME (SEQ ID NO: 2) contains the 5′ UTR, 3′ UTR, and non-structural proteins derived from the attenuated TC-83 strain of VEEV, with the aforementioned Zika virus genes replacing the VEEV structural proteins downstream of a T7 subgenomic promoter as shown in FIG. 7A. The antibiotic resistance gene to Ampicillin used in J. H. Erasmus supra was changed to Kanamycin to allow for GMP manufacture. The subgenomic promoter was optimized for antigen expression enhancement by changing the sequence from gccgccgcc to tagtccgccaag (SEQ ID NO: 3). Otherwise, the plasmid pT7-VEE-Zika-prME is identical to the plasmid described in J. H. Erasmus supra.

Similarly, DNA templates for saRNA encoding the secreted alkaline phosphatase protein (SEAP) were constructed in two different versions. The first, pT7-VEE-SEAP-V1 (SEQ ID NO: 4) shown in FIG. 7B, is identical to that described in J. H. Erasmus supra. This plasmid was the template for all SEAP-saRNA used in the long-term stability studies shown in FIG. 5. An updated version (pT7-VEE-SEAP-V2 (SEQ ID NO: 5) in FIG. 7C) reflects the same antibiotic resistance gene and subgenomic promoter changes described above to allow for direct comparison to pT7-VEE-Zika-prME plasmid in the vaccine immunogenicity studies in FIG. 3. All plasmid sequences were confirmed using Sanger sequencing. DNA templates were amplified in E. coli and isolated using maxi or gigaprep kits (Qiagen) and linearized by NotI restriction digest (New England Biolabs). Linearized DNA was purified by phenol chloroform extraction.

DNA plasmid encoding the SARS-CoV2 spike was produced in the same manner as the plasmid encoding the Zika proteins. This plasmid (SEQ ID NO: 6) is shown in a linear representation in FIG. 7D. The SARS-CoV2 spike open reading frame sequence (GenBank MT246667.1|(SEQ ID NO: 7) was used as a template, additionally incorporating the D614G mutation and substitution of PP for KV at amino acid positions 987-988 and the addition of nine N-terminal codons encoding amino acid sequence MFLLTTKRT (SEQ ID NO: 8)(which are also encoded in the reference genome). This sequence was then codon-optimized for mammalian expression, synthesized by BioXp and inserted into the TC-83 strain of VEEV backbone expression vector by Gibson cloning.

RNA Production and Purification

Generation of saRNA stocks was achieved by T7 promoter-mediated in vitro transcription using NotI-linearized DNA template. saRNA was manufactured with a standard in vitro transcription protocol using T7 polymerase, RNase inhibitor, and pyrophosphatase enzymes (Aldevron). DNA plasmid was digested away (DNase I, Aldevron) and cap0 structures were added to the transcripts by vaccinia capping enzyme, GTP, and S-adenosyl-methionine (Aldevron). RNA was then purified from the transcription and capping reaction components by chromatography using a CaptoCore 700 resin (GE Healthcare) followed by diafiltration and concentration using tangential flow filtration. The saRNA material was terminally filtered with a 0.22 μm polyethersulfone filter and stored at −80° C. until use. All saRNA was characterized by agarose gel electrophoresis and quantified both by UV absorbance (NanoDrop 1000) and Ribogreen assay (Thermo Fisher).

### SEQUENCES

Sequences discussed in this disclosure are included below.

codon optimized ZIKV strain H/PF/2013 prM and E genes
SEQ ID NO: 1
atgcggagaggagcagacacatccgtgggaatcgtgggcctgctgctgaccacagcaatggcagccgaggtga
ccaggagaggcagcgcctactatatgtacctggacagaaatgatgccggcgaggccatctcctttcccaccacactgggcatga
acaagtgctacatccagatcatggacctgggccacatgtgcgatgccaccatgagctatgagtgtccaatgctggacgagggcgt
ggagcccgacgatgtggattgctggtgtaataccacatctacatgggtggtgtacggcacctgtcaccacaagaagggagaggc
acggcgcagcaggagagcagtgacactgccttcccactctaccaggaagctgcagacaagaagccagacctggctggagtcc
agggagtatacaaagcacctgatcagggtggagaactggatctttagaaatccaggattcgcactggctgccgccgccatcgcat
ggctgctgggcagctccaccagccagaaagtgatctacctggtcatgatcctgctgatcgcccctgcctattctatccggtgcatcg
gcgtgagcaatagggacttcgtggagggaatgtccggaggcacctgggtggatgtggtgctggagcacggcggctgcgtgac
agtgatggcccaggacaagccaaccgtggatatcgagctggtgaccacaaccgtgtccaacatggccgaggtgaggtcttactg
ctatgaggccagcatctccgacatggcctctgatagcagatgtcccacccagggcgaggcctacctggacaagcagtccgatac
acagtacgtgtgcaagcggaccctggtggacaggggatggggaaatggatgtggcctgtttggcaagggctctctggtgacatg
cgccaagttcgcctgtagcaagaagatgaccggcaagtccatccagccagagaacctggagtaccggatcatgctgtctgtgca
cggctcccagcactctggcatgatcgtgaacgacacaggccacgagacagatgagaatcgggccaaggtggagatcacaccta
actccccacgcgccgaggccaccctgggaggatttggctctctgggcctggactgcgagcctaggacaggcctggacttctccg
atctgtactatctgaccatgaacaataagcactggctggtgcacaaggagtggtttcacgacatcccactgccatggcacgcagga
gcagatacaggcaccccacactggaacaataaggaggccctggtggagttcaaggacgcccacgccaagcggcagacagtg
gtggtgctgggcagccaggagggagcagtgcacaccgccctggcaggcgccctggaggccgagatggacggagcaaaggg
ccgcctgtctagcggccacctgaagtgcaggctgaagatggataagctgagactgaagggcgtgtcctactctctgtgcacagcc
gccttcaccttcaccaagatccctgccgagacactgcacggcacagtgaccgtggaggtgcagtatgccggcacagacggccc
ctgtaaggtgcctgcccagatggccgtggatatgcagacactgacccctgtgggcaggctgatcaccgccaatccagtgatcac
agagtctaccgagaacagcaagatgatgctggagctggacccccctttcggcgatagctatatcgtgatcggcgtgggcgagaa
gaagatcacacaccactggcacagaagcggctccacaatcggcaaggcctttgaggcaaccgtgcggggagcaaagaggatg
gccgtgctgggcgacaccgcatgggatttcggatctgtgggaggcgccctgaacagcctgggcaagggcatccaccagatctt
cggcgccgcctttaagtccctgttcggcggcatgagctggttttcccagatcctgatcggcacactgctgatgtggctgggcctga
acaccaagaatggctctatcagcctgatgtgcctggccctgggaggcgtgctgatcttcctgtccaccgccgtgtctgcc
plasmid pT7-VEE-Zika-prME
SEQ ID NO: 2
ataggcggcgcatgagagaagcccagaccaattacctacccaaaatggagaaagttcacgttgacatcgaggaag
acagcccattcctcagagctttgcagcggagcttcccgcagtttgaggtagaagccaagcaggtcactgataatgaccatgctaat
gccagagcgttttcgcatctggcttcaaaactgatcgaaacggaggtggacccatccgacacgatccttgacattggaagtgcgc
ccgcccgcagaatgtattctaagcacaagtatcattgtatctgtccgatgagatgtgcggaagatccggacagattgtataagtatg
caactaagctgaagaaaaactgtaaggaaataactgataaggaattggacaagaaaatgaaggagctggccgccgtcatgagc
gaccctgacctggaaactgagactatgtgcctccacgacgacgagtcgtgtcgctacgaagggcaagtcgctgtttaccaggatg
tatacgcggttgacggaccgacaagtctctatcaccaagccaataagggagttagagtcgcctactggataggctttgacaccacc
ccttttatgtttaagaacttggctggagcatatccatcatactctaccaactgggccgacgaaaccgtgttaacggctcgtaacatag
gcctatgcagctctgacgttatggagcggtcacgtagagggatgtccattcttagaaagaagtatttgaaaccatccaacaatgttct
attctctgttggctcgaccatctaccacgagaagagggacttactgaggagctggcacctgccgtctgtatttcacttacgtggcaa
gcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggt
ctcttttcccgtgtgcacgtatgtgccagctacattgtgtgaccaaatgactggcatactggcaacagatgtcagtgcggacgacgc
gcaaaaactgctggttgggctcaaccagcgtatagtcgtcaacggtcgcacccagagaaacaccaataccatgaaaaattaccttt
tgcccgtagtggcccaggcatttgctaggtgggcaaaggaatataaggaagatcaagaagatgaaaggccactaggactacga
gatagacagttagtcatggggtgttgttgggcttttagaaggcacaagataacatctatttataagcgcccggatacccaaaccatc
atcaaagtgaacagcgatttccactcattcgtgctgcccaggataggcagtaacacattggagatcgggctgagaacaagaatca
ggaaaatgttagaggagcacaaggagccgtcacctctcattaccgccgaggacgtacaagaagctaagtgcgcagccgatgag
gctaaggaggtgcgtgaagccgaggagttgcgcgcagctctaccacctttggcagctgatgttgaggagcccactctggaggca
gacgtcgacttgatgttacaagaggctggggccggctcagtggagacacctcgtggcttgataaaggttaccagctacgatggcg
aggacaagatcggctcttacgctgtgctttctccgcaggctgtactcaagagtgaaaaattatcttgcatccaccctctcgctgaaca
agtcatagtgataacacactctggccgaaaagggcgttatgccgtggaaccataccatggtaaagtagtggtgccagagggacat
gcaatacccgtccaggactttcaagctctgagtgaaagtgccaccattgtgtacaacgaacgtgagttcgtaaacaggtacctgca
ccatattgccacacatggaggagcgctgaacactgatgaagaatattacaaaactgtcaagcccagcgagcacgacggcgaata
cctgtacgacatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtatggcgtgcca
ggatcaggcaagtctggcatcattaaaagcgcagtcaccaaaaaagatctagtggtgagcgccaagaaagaaaactgtgcagaa
attataagggacgtcaagaaaatgaaagggctggacgtcaatgccagaactgtggactcagtgctcttgaatggatgcaaacacc
ccgtagagaccctgtatattgacgaagcttttgcttgtcatgcaggtactctcagagcgctcatagccattataagacctaaaaaggc
agtgctctgcggggatcccaaacagtgcggtttttttaacatgatgtgcctgaaagtgcattttaaccacgagatttgcacacaagtct
tccacaaaagcatctctcgccgttgcactaaatctgtgacttcggtcgtctcaaccttgttttacgacaaaaaaatgagaacgacgaa
tccgaaagagactaagattgtgattgacactaccggcagtaccaaacctaagcaggacgatctcattctcacttgtttcagagggtg
ggtgaagcagttgcaaatagattacaaaggcaacgaaataatgacggcagctgcctctcaagggctgacccgtaaaggtgtgtat
gccgttcggtacaaggtgaatgaaaatcctctgtacgcacccacctcagaacatgtgaacgtcctactgacccgcacggaggacc
gcatcgtgtggaaaacactagccggcgacccatggataaaaacactgactgccaagtaccctgggaatttcactgccacgataga
ggagtggcaagcagagcatgatgccatcatgaggcacatcttggagagaccggaccctaccgacgtcttccagaataaggcaa
acgtgtgttgggccaaggctttagtgccggtgctgaagaccgctggcatagacatgaccactgaacaatggaacactgtggattat
tttgaaacggacaaagctcactcagcagagatagtattgaaccaactatgcgtgaggttctttggactcgatctggactccggtctat
tttctgcacccactgttccgttatccattaggaataatcactgggataactccccgtcgcctaacatgtacgggctgaataaagaagt
ggtccgtcagctctctcgcaggtacccacaactgcctcgggcagttgccactggaagagtctatgacatgaacactggtacactg
cgcaattatgatccgcgcataaacctagtacctgtaaacagaagactgcctcatgctttagtcctccaccataatgaacacccacag
agtgacttttcttcattcgtcagcaaattgaagggcagaactgtcctggtggtcggggaaaagttgtccgtcccaggcaaaatggtt
120=gactggttgtcagaccggcctgaggctaccttcagagctcggctggatttaggcatcccaggtgatgtgcccaaatatgacataat
atttgttaatgtgaggaccccatataaataccatcactatcagcagtgtgaagaccatgccattaagcttagcatgttgaccaagaaa
gcttgtctgcatctgaatcccggcggaacctgtgtcagcataggttatggttacgctgacagggccagcgaaagcatcattggtgc
tatagcgcggcagttcaagttttcccgggtatgcaaaccgaaatcctcacttgaagagacggaagttctgtttgtattcattgggtac
gatcgcaaggcccgtacgcacaatccttacaagctttcatcaaccttgaccaacatttatacaggttccagactccacgaagccgg
atgtgcaccctcatatcatgtggtgcgaggggatattgccacggccaccgaaggagtgattataaatgctgctaacagcaaagga
caacctggcggaggggtgtgcggagcgctgtataagaaattcccggaaagcttcgatttacagccgatcgaagtaggaaaagcg
cgactggtcaaaggtgcagctaaacatatcattcatgccgtaggaccaaacttcaacaaagtttcggaggttgaaggtgacaaaca
gttggcagaggcttatgagtccatcgctaagattgtcaacgataacaattacaagtcagtagcgattccactgttgtccaccggcatc
ttttccgggaacaaagatcgactaacccaatcattgaaccatttgctgacagctttagacaccactgatgcagatgtagccatatact
gcagggacaagaaatgggaaatgactctcaaggaagcagtggctaggagagaagcagtggaggagatatgcatatccgacga
ctcttcagtgacagaacctgatgcagagctggtgagggtgcatccgaagagttctttggctggaaggaagggctacagcacaag
cgatggcaaaactttctcatatttggaagggaccaagtttcaccaggcggccaaggatatagcagaaattaatgccatgtggcccg
ttgcaacggaggccaatgagcaggtatgcatgtatatcctcggagaaagcatgagcagtattaggtcgaaatgccccgtcgaaga
gtcggaagcctccacaccacctagcacgctgccttgcttgtgcatccatgccatgactccagaaagagtacagcgcctaaaagcc
tcacgtccagaacaaattactgtgtgctcatcctttccattgccgaagtatagaatcactggtgtgcagaagatccaatgctcccagc
ctatattgttctcaccgaaagtgcctgcgtatattcatccaaggaagtatctcgtggaaacaccaccggtagacgagactccggag
ccatcggcagagaaccaatccacagaggggacacctgaacaaccaccacttataaccgaggatgagaccaggactagaacgc
ctgagccgatcatcatcgaagaggaagaagaggatagcataagtttgctgtcagatggcccgacccaccaggtgctgcaagtcg
aggcagacattcacgggccgccctctgtatctagctcatcctggtccattcctcatgcatccgactttgatgtggacagtttatccata
cttgacaccctggagggagctagcgtgaccagcggggcaacgtcagccgagactaactcttacttcgcaaagagtatggagtttc
tggcgcgaccggtgcctgcgcctcgaacagtattcaggaaccctccacatcccgctccgcgcacaagaacaccgtcacttgcac
ccagcagggcctgctcgagaaccagcctagtttccaccccgccaggcgtgaatagggtgatcactagagaggagctcgaggcg
cttaccccgtcacgcactcctagcaggtcggtctcgagaaccagcctggtctccaacccgccaggcgtaaatagggtgattacaa
gagaggagtttgaggcgttcgtagcacaacaacaatgacggtttgatgcgggtgcatacatcttttcctccgacaccggtcaaggg
catttacaacaaaaatcagtaaggcaaacggtgctatccgaagtggtgttggagaggaccgaattggagatttcgtatgccccgcg
cctcgaccaagaaaaagaagaattactacgcaagaaattacagttaaatcccacacctgctaacagaagcagataccagtccagg
aaggtggagaacatgaaagccataacagctagacgtattctgcaaggcctagggcattatttgaaggcagaaggaaaagtggag
tgctaccgaaccctgcatcctgttcctttgtattcatctagtgtgaaccgtgccttttcaagccccaaggtcgcagtggaagcctgtaa
cgccatgttgaaagagaactttccgactgtggcttcttactgtattattccagagtacgatgcctatttggacatggttgacggagcttc
atgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgaga
gaattgcccgtattggattcggcggcctttaatgtggaatgcttcaagaaatatgcgtgtaataatgaatattgggaaacgtttaaaga
aaaccccatcaggcttactgaagaaaacgtggtaaattacattaccaaattaaaaggaccaaaagctgctgctctttttgcgaagac
acataatttgaatatgttgcaggacataccaatggacaggtttgtaatggacttaaagagagacgtgaaagtgactccaggaacaa
aacatactgaagaacggcccaaggtacaggtgatccaggctgccgatccgctagcaacagcgtatctgtgcggaatccaccga
gagctggttaggagattaaatgcggtcctgcttccgaacattcatacactgtttgatatgtcggctgaagactttgacgctattatagc
cgagcacttccagcctggggattgtgttctggaaactgacatcgcgtcgtttgataaaagtgaggacgacgccatggctctgaccg
cgttaatgattctggaagacttaggtgtggacgcagagctgttgacgctgattgaggcggctttcggcgaaatttcatcaatacattt
gcccactaaaactaaatttaaattcggagccatgatgaaatctggaatgttcctcacactgtttgtgaacacagtcattaacattgtaat
cgcaagcagagtgttgagagaacggctaaccggatcaccatgtgcagcattcattggagatgacaatatcgtgaaaggagtcaa
atcggacaaattaatggcagacaggtgcgccacctggttgaatatggaagtcaagattatagatgctgtggtgggcgagaaagcg
ccttatttctgtggagggtttattttgtgtgactccgtgaccggcacagcgtgccgtgtggcagaccccctaaaaaggctgtttaagc
ttggcaaacctctggcagcagacgatgaacatgatgatgacaggagaagggcattgcatgaagagtcaacacgctggaaccga
gtgggtattctttcagagctgtgcaaggcagtagaatcaaggtatgaaaccgtaggaacttccatcatagttatggccatgactactc
tagctagcagtgttaaatcattcagctacctgagaggggcccctataactctctacggctaacctgaatggactacgacatagtcta
gtccgccaagatgcggagaggagcagacacatccgtgggaatcgtgggcctgctgctgaccacagcaatggcagccgaggtg
accaggagaggcagcgcctactatatgtacctggacagaaatgatgccggcgaggccatctcctttcccaccacactgggcatg
aacaagtgctacatccagatcatggacctgggccacatgtgcgatgccaccatgagctatgagtgtccaatgctggacgagggc
gtggagcccgacgatgtggattgctggtgtaataccacatctacatgggtggtgtacggcacctgtcaccacaagaagggagag
gcacggcgcagcaggagagcagtgacactgccttcccactctaccaggaagctgcagacaagaagccagacctggctggagt
ccagggagtatacaaagcacctgatcagggtggagaactggatctttagaaatccaggattcgcactggctgccgccgccatcg
catggctgctgggcagctccaccagccagaaagtgatctacctggtcatgatcctgctgatcgcccctgcctattctatccggtgca
tcggcgtgagcaatagggacttcgtggagggaatgtccggaggcacctgggtggatgtggtgctggagcacggcggctgcgtg
acagtgatggcccaggacaagccaaccgtggatatcgagctggtgaccacaaccgtgtccaacatggccgaggtgaggtcttac
tgctatgaggccagcatctccgacatggcctctgatagcagatgtcccacccagggcgaggcctacctggacaagcagtccgat
acacagtacgtgtgcaagcggaccctggtggacaggggatggggaaatggatgtggcctgtttggcaagggctctctggtgaca
tgcgccaagttcgcctgtagcaagaagatgaccggcaagtccatccagccagagaacctggagtaccggatcatgctgtctgtg
cacggctcccagcactctggcatgatcgtgaacgacacaggccacgagacagatgagaatcgggccaaggtggagatcacac
ctaactccccacgcgccgaggccaccctgggaggatttggctctctgggcctggactgcgagcctaggacaggcctggacttct
ccgatctgtactatctgaccatgaacaataagcactggctggtgcacaaggagtggtttcacgacatcccactgccatggcacgca
ggagcagatacaggcaccccacactggaacaataaggaggccctggtggagttcaaggacgcccacgccaagcggcagaca
gtggtggtgctgggcagccaggagggagcagtgcacaccgccctggcaggcgccctggaggccgagatggacggagcaaa
gggccgcctgtctagcggccacctgaagtgcaggctgaagatggataagctgagactgaagggcgtgtcctactctctgtgcac
agccgccttcaccttcaccaagatccctgccgagacactgcacggcacagtgaccgtggaggtgcagtatgccggcacagacg
gcccctgtaaggtgcctgcccagatggccgtggatatgcagacactgacccctgtgggcaggctgatcaccgccaatccagtga
tcacagagtctaccgagaacagcaagatgatgctggagctggacccccctttcggcgatagctatatcgtgatcggcgtgggcga
gaagaagatcacacaccactggcacagaagcggctccacaatcggcaaggcctttgaggcaaccgtgcggggagcaaagag
gatggccgtgctgggcgacaccgcatgggatttcggatctgtgggaggcgccctgaacagcctgggcaagggcatccaccag
atcttcggcgccgcctttaagtccctgttcggcggcatgagctggttttcccagatcctgatcggcacactgctgatgtggctgggc
ctgaacaccaagaatggctctatcagcctgatgtgcctggccctgggaggcgtgctgatcttcctgtccaccgccgtgtctgcctg
accgcggtgtcaaaaaccgcgtggacgtggttaacatccctgctgggaggatcagccgtaattattataattggcttggtgctggct
actattgtggccatgtacgtgctgaccaaccagaaacataattgaatacagcagcaattggcaagctgcttacatagaactcgcgg
cgattggcatgccgccttaaaatttttattttattttttcttttcttttccgaatcggattttgtttttaatatttcaaaaaaaaaaaaaaaaaaa
aaaaaaaaaaaaaaaaaaaaaaaaaagcggccgcgagcttggctcgagcctcgagcatggtcatagctgtttcctgtgtgaaatt
gttatccgctcacaattccacacaacatacgagccggaagcataaagtgtaaagcctggggtgcctaatgagtgagctaactcac
attaattgcgttgcgctcactgcccgctttccagtcgggaaacctgtcgtgccagctgcattaatgaatcggccaacgcgcgggga
gaggcggtttgcgtattgggcgctcttccgcttcctcgctcactgactcgctgcgctcggtcgttcggctgcggcgagcggtatca
gctcactcaaaggcggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcacaaaatcacaaaaatc
gacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcatagctcacgctgtaggtat
ctcagttcggtgtaggtcgttcgctccaagctgggctgtgtgcacgaaccccccgttcagcccgaccgctgcgccttatccggtaa
ctatcgtcttgagtccaacccggtaagacacgacttatcgccactggcagcagccactggtaacaggattagcagagcgaggtat
gtaggcggtgctacagagttcttgaagtggtggcctaactacggctacactagaagaacagtatttggtatctgcgctctgctgaag
ccagttaccttcggaaaaagagttggtagctcttgatccggcaaacaaaccaccgctggtagcggtggtttttttgtttgcaagcagc
agattacgcgcagaaaaaaaggatctcaagaagatcctttgatcttttctacggggtctgacgctcagtggaacgaaaactcacgtt
aagggattttggtcatgagattatcaaaaaggatcttcacctagatccttttaaattaaaaatgaagttttaaatcaatctaaagtatatat
gagtaaacttggtctgacagttagaaaaactcatcgagcatcaaatgaaactgcaatttattcatatcaggattatcaataccatattttt
gaaaaagccgtttctgtaatgaaggagaaaactcaccgaggcagttccataggatggcaagatcctggtatcggtctgcgattccg
actcgtccaacatcaatacaacctattaatttcccctcgtcaaaaataaggttatcaagtgagaaatcaccatgagtgacgactgaat
ccggtgagaatggcaaaagtttatgcatttctttccagacttgttcaacaggccagccattacgctcgtcatcaaaatcactcgcatc
aaccaaaccgttattcattcgtgattgcgcctgagcgagacgaaatacgcgatcgctgttaaaaggacaattacaaacaggaatcg
aatgcaaccggcgcaggaacactgccagcgcatcaacaatattttcacctgaatcaggatattcttctaatacctggaatgctgtttt
cccagggatcgcagtggtgagtaaccatgcatcatcaggagtacggataaaatgcttgatggtcggaagaggcataaattccgtc
agccagtttagtctgaccatctcatctgtaacatcattggcaacgctacctttgccatgtttcagaaacaactctggcgcatcgggctt
cccatacaatcgatagattgtcgcacctgattgcccgacattatcgcgagcccatttatacccatataaatcagcatccatgttggaat
ttaatcgcggcctagagcaagacgtttcccgttgaatatggctcatactcttcctttttcaatattattgaagcatttatcagggttattgt
ctcatgagcggatacatatttgaatgtatttagaaaaataaacaaataggggttccgcgcacatttccccgaaaagtgccacctgac
gtctaagaaaccattattatcatgacattaacctataaaaataggcgtatcacgaggccctttcgtctcgcgcgtttcggtgatgacg
gtgaaaacctctgacacatgcagctcccggagacggtcacagcttgtctgtaagcggatgccgggagcagacaagcccgtcag
ggcgcgtcagcgggtgttggcgggtgtcggggctggcttaactatgcggcatcagagcagattgtactgagagtgcaccatatg
cggtgtgaaataccgcacagatgcgtaaggagaaaataccgcatcaggcgccattcgccattcaggctgcgcaactgttgggaa
gggcgatcggtgcgggcctcttcgctattacgccagctggcgaaagggggatgtgctgcaaggcgattaagttgggtaacgcca
gggttttcccagtcacgacgttgtaaaacgacggccagtgaattgacgcgttaatacgactcactatag
T7 subgenomic promoter
SEQ ID NO: 3
tagtccgccaag
plasmid pT7-VEE-SEAP-V1
SEQ ID NO: 4
ataggcggcgcatgagagaagcccagaccaattacctacccaaaatggagaaagttcacgttgacatcgaggaag
acagcccattcctcagagctttgcagcggagcttcccgcagtttgaggtagaagccaagcaggtcactgataatgaccatgctaat
gccagagcgttttcgcatctggcttcaaaactgatcgaaacggaggtggacccatccgacacgatccttgacattggaagtgcgc
ccgcccgcagaatgtattctaagcacaagtatcattgtatctgtccgatgagatgtgcggaagatccggacagattgtataagtatg
caactaagctgaagaaaaactgtaaggaaataactgataaggaattggacaagaaaatgaaggagctggccgccgtcatgagc
gaccctgacctggaaactgagactatgtgcctccacgacgacgagtcgtgtcgctacgaagggcaagtcgctgtttaccaggatg
tatacgcggttgacggaccgacaagtctctatcaccaagccaataagggagttagagtcgcctactggataggctttgacaccacc
ccttttatgtttaagaacttggctggagcatatccatcatactctaccaactgggccgacgaaaccgtgttaacggctcgtaacatag
gcctatgcagctctgacgttatggagcggtcacgtagagggatgtccattcttagaaagaagtatttgaaaccatccaacaatgttct
attctctgttggctcgaccatctaccacgagaagagggacttactgaggagctggcacctgccgtctgtatttcacttacgtggcaa
gcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggt
ctcttttcccgtgtgcacgtatgtgccagctacattgtgtgaccaaatgactggcatactggcaacagatgtcagtgcggacgacgc
gcaaaaactgctggttgggctcaaccagcgtatagtcgtcaacggtcgcacccagagaaacaccaataccatgaaaaattaccttt
tgcccgtagtggcccaggcatttgctaggtgggcaaaggaatataaggaagatcaagaagatgaaaggccactaggactacga
gatagacagttagtcatggggtgttgttgggcttttagaaggcacaagataacatctatttataagcgcccggatacccaaaccatc
atcaaagtgaacagcgatttccactcattcgtgctgcccaggataggcagtaacacattggagatcgggctgagaacaagaatca
ggaaaatgttagaggagcacaaggagccgtcacctctcattaccgccgaggacgtacaagaagctaagtgcgcagccgatgag
gctaaggaggtgcgtgaagccgaggagttgcgcgcagctctaccacctttggcagctgatgttgaggagcccactctggaggca
gacgtcgacttgatgttacaagaggctggggccggctcagtggagacacctcgtggcttgataaaggttaccagctacgatggcg
aggacaagatcggctcttacgctgtgctttctccgcaggctgtactcaagagtgaaaaattatcttgcatccaccctctcgctgaaca
agtcatagtgataacacactctggccgaaaagggcgttatgccgtggaaccataccatggtaaagtagtggtgccagagggacat
gcaatacccgtccaggactttcaagctctgagtgaaagtgccaccattgtgtacaacgaacgtgagttcgtaaacaggtacctgca
ccatattgccacacatggaggagcgctgaacactgatgaagaatattacaaaactgtcaagcccagcgagcacgacggcgaata
cctgtacgacatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtatggcgtgcca
ggatcaggcaagtctggcatcattaaaagcgcagtcaccaaaaaagatctagtggtgagcgccaagaaagaaaactgtgcagaa
attataagggacgtcaagaaaatgaaagggctggacgtcaatgccagaactgtggactcagtgctcttgaatggatgcaaacacc
ccgtagagaccctgtatattgacgaagcttttgcttgtcatgcaggtactctcagagcgctcatagccattataagacctaaaaaggc
agtgctctgcggggatcccaaacagtgcggtttttttaacatgatgtgcctgaaagtgcattttaaccacgagatttgcacacaagtct
tccacaaaagcatctctcgccgttgcactaaatctgtgacttcggtcgtctcaaccttgttttacgacaaaaaaatgagaacgacgaa
tccgaaagagactaagattgtgattgacactaccggcagtaccaaacctaagcaggacgatctcattctcacttgtttcagagggtg
ggtgaagcagttgcaaatagattacaaaggcaacgaaataatgacggcagctgcctctcaagggctgacccgtaaaggtgtgtat
gccgttcggtacaaggtgaatgaaaatcctctgtacgcacccacctcagaacatgtgaacgtcctactgacccgcacggaggacc
gcatcgtgtggaaaacactagccggcgacccatggataaaaacactgactgccaagtaccctgggaatttcactgccacgataga
ggagtggcaagcagagcatgatgccatcatgaggcacatcttggagagaccggaccctaccgacgtcttccagaataaggcaa
acgtgtgttgggccaaggctttagtgccggtgctgaagaccgctggcatagacatgaccactgaacaatggaacactgtggattat
tttgaaacggacaaagctcactcagcagagatagtattgaaccaactatgcgtgaggttctttggactcgatctggactccggtctat
tttctgcacccactgttccgttatccattaggaataatcactgggataactccccgtcgcctaacatgtacgggctgaataaagaagt
ggtccgtcagctctctcgcaggtacccacaactgcctcgggcagttgccactggaagagtctatgacatgaacactggtacactg
cgcaattatgatccgcgcataaacctagtacctgtaaacagaagactgcctcatgctttagtcctccaccataatgaacacccacag
agtgacttttcttcattcgtcagcaaattgaagggcagaactgtcctggtggtcggggaaaagttgtccgtcccaggcaaaatggtt
gactggttgtcagaccggcctgaggctaccttcagagctcggctggatttaggcatcccaggtgatgtgcccaaatatgacataat
atttgttaatgtgaggaccccatataaataccatcactatcagcagtgtgaagaccatgccattaagcttagcatgttgaccaagaaa
gcttgtctgcatctgaatcccggcggaacctgtgtcagcataggttatggttacgctgacagggccagcgaaagcatcattggt
tatagcgcggcagttcaagttttcccgggtatgcaaaccgaaatcctcacttgaagagacggaagttctgtttgtattcattgggtac
gatcgcaaggcccgtacgcacaatccttacaagctttcatcaaccttgaccaacatttatacaggttccagactccacgaagccgg
atgtgcaccctcatatcatgtggtgcgaggggatattgccacggccaccgaaggagtgattataaatgctgctaacagcaaagga
caacctggcggaggggtgtgcggagcgctgtataagaaattcccggaaagcttcgatttacagccgatcgaagtaggaaaagcg
cgactggtcaaaggtgcagctaaacatatcattcatgccgtaggaccaaacttcaacaaagtttcggaggttgaaggtgacaaaca
gttggcagaggcttatgagtccatcgctaagattgtcaacgataacaattacaagtcagtagcgattccactgttgtccaccggcatc
ttttccgggaacaaagatcgactaacccaatcattgaaccatttgctgacagctttagacaccactgatgcagatgtagccatatact
gcagggacaagaaatgggaaatgactctcaaggaagcagtggctaggagagaagcagtggaggagatatgcatatccgacga
ctcttcagtgacagaacctgatgcagagctggtgagggtgcatccgaagagttctttggctggaaggaagggctacagcacaag
cgatggcaaaactttctcatatttggaagggaccaagtttcaccaggcggccaaggatatagcagaaattaatgccatgtggcccg
ttgcaacggaggccaatgagcaggtatgcatgtatatcctcggagaaagcatgagcagtattaggtcgaaatgccccgtcgaaga
gtcggaagcctccacaccacctagcacgctgccttgcttgtgcatccatgccatgactccagaaagagtacagcgcctaaaagcc
tcacgtccagaacaaattactgtgtgctcatcctttccattgccgaagtatagaatcactggtgtgcagaagatccaatgctcccagc
ctatattgttctcaccgaaagtgcctgcgtatattcatccaaggaagtatctcgtggaaacaccaccggtagacgagactccggag
ccatcggcagagaaccaatccacagaggggacacctgaacaaccaccacttataaccgaggatgagaccaggactagaacgc
ctgagccgatcatcatcgaagaggaagaagaggatagcataagtttgctgtcagatggcccgacccaccaggtgctgcaagtcg
aggcagacattcacgggccgccctctgtatctagctcatcctggtccattcctcatgcatccgactttgatgtggacagtttatccata
cttgacaccctggagggagctagcgtgaccagcggggcaacgtcagccgagactaactcttacttcgcaaagagtatggagtttc
tggcgcgaccggtgcctgcgcctcgaacagtattcaggaaccctccacatcccgctccgcgcacaagaacaccgtcacttgcac
ccagcagggcctgctcgagaaccagcctagtttccaccccgccaggcgtgaatagggtgatcactagagaggagctcgaggcg
cttaccccgtcacgcactcctagcaggtcggtctcgagaaccagcctggtctccaacccgccaggcgtaaatagggtgattacaa
gagaggagtttgaggcgttcgtagcacaacaacaatgacggtttgatgcgggtgcatacatcttttcctccgacaccggtcaaggg
catttacaacaaaaatcagtaaggcaaacggtgctatccgaagtggtgttggagaggaccgaattggagatttcgtatgccccgcg
cctcgaccaagaaaaagaagaattactacgcaagaaattacagttaaatcccacacctgctaacagaagcagataccagtccagg
aaggtggagaacatgaaagccataacagctagacgtattctgcaaggcctagggcattatttgaaggcagaaggaaaagtggag
tgctaccgaaccctgcatcctgttcctttgtattcatctagtgtgaaccgtgccttttcaagccccaaggtcgcagtggaagcctgtaa
cgccatgttgaaagagaactttccgactgtggcttcttactgtattattccagagtacgatgcctatttggacatggttgacggagcttc
atgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgaga
gaattgcccgtattggattcggcggcctttaatgtggaatgcttcaagaaatatgcgtgtaataatgaatattgggaaacgtttaaaga
aaaccccatcaggcttactgaagaaaacgtggtaaattacattaccaaattaaaaggaccaaaagctgctgctctttttgcgaagac
acataatttgaatatgttgcaggacataccaatggacaggtttgtaatggacttaaagagagacgtgaaagtgactccaggaacaa
aacatactgaagaacggcccaaggtacaggtgatccaggctgccgatccgctagcaacagcgtatctgtgcggaatccaccga
gagctggttaggagattaaatgcggtcctgcttccgaacattcatacactgtttgatatgtcggctgaagactttgacgctattatagc
cgagcacttccagcctggggattgtgttctggaaactgacatcgcgtcgtttgataaaagtgaggacgacgccatggctctgaccg
cgttaatgattctggaagacttaggtgtggacgcagagctgttgacgctgattgaggcggctttcggcgaaatttcatcaatacattt
gcccactaaaactaaatttaaattcggagccatgatgaaatctggaatgttcctcacactgtttgtgaacacagtcattaacattgtaat
cgcaagcagagtgttgagagaacggctaaccggatcaccatgtgcagcattcattggagatgacaatatcgtgaaaggagtcaa
atcggacaaattaatggcagacaggtgcgccacctggttgaatatggaagtcaagattatagatgctgtggtgggcgagaaagcg
ccttatttctgtggagggtttattttgtgtgactccgtgaccggcacagcgtgccgtgtggcagaccccctaaaaaggctgtttaagc
ttggcaaacctctggcagcagacgatgaacatgatgatgacaggagaagggcattgcatgaagagtcaacacgctggaaccga
gtgggtattctttcagagctgtgcaaggcagtagaatcaaggtatgaaaccgtaggaacttccatcatagttatggccatgactactc
tagctagcagtgttaaatcattcagctacctgagaggggcccctataactctctacggctaacctgaatggactacgacatagtcta
gtccgccaagatgctcctcttgctccttctcttgggactccgattgcagctctctttgggtataattcctgtcgaagaagagaatcccg
acttctggaatcgcgaagcggcagaagcattgggtgcggcaaaaaagctccaaccggcacagactgcggccaagaatctcata
attttcttgggtgatggcatgggcgtttccactgtgactgcggcacgaatcctgaagggtcaaaagaaggataaactgggtccaga
gatcccgctggctatggataggtttccatacgttgcgctctctaagacctataacgtcgataagcatgtaccagactccggagcgac
ggccactgcttacctttgtggagttaaaggtaatttccaaacaataggactgagcgcagctgcaagattcaaccaatgcaacacga
caagagggaatgaggtgatttcagtcatgaatcgagcaaagaaagctgggaaatccgttggcgtggtcaccactacaagagtgc
agcatgcatctccagcaggaacttacgcccacactgtaaacagaaactggtatagtgacgccgatgttcccgcctctgcgaggca
agagggttgtcaagacatcgctacccagctcataagcaacatggacattgatgtaatactgggtgggggtcggaaatacatgttcc
ggatgggcactcccgatccggagtacccggatgactattcccaaggaggtaccagattggacgggaaaaatcttgtccaagaat
ggctcgccaagcggcagggggcaaggtacgtgtggaacaggacagaactgatgcaggcaagcttggatccaagcgtaacgc
atcttatgggtctttttgaacccggtgatatgaaatacgaaatacatcgcgactcaacactggacccgtctctcatggagatgactga
agctgccttgaggttgttgagtcggaaccctaggggctttttcttgttcgtagaggggggcgaattgaccacggtcatcacgaatc
tcgagcgtaccgggcgctcacagaaaccatcatgtttgacgatgctatcgaacgagcgggtcagcttacctctgaagaagatacg
ctctctcttgtcaccgcggaccatagccatgttttttccttcggtggttatccgttgcgaggttccagcatattcggcctcgcgccagg
gaaagcccgcgaccgcaaagcttatacggtgctgctttacggaaacggccctggttacgtccttaaagacggtgcgagacctga
cgtgacggaatctgaatccggttctcccgaatatagacaacagagtgctgttccgctggatgaagagactcatgcgggagaagat
gtagcggtttttgctagggggccgcaagcacaccttgttcatggcgttcaggagcaaactttcatagcccatgtaatggcatttgctg
cgtgtctcgagccgtataccgcttgcgatctcgctccgccggcgggtacaaccgatgctgcccacccggggtactcaagagtag
gggcagcagggcgatttgaacaaacttgataaggcgcgccatacagcagcaattggcaagctgcttacatagaactcgcggcga
ttggcatgccgccttaaaatttttattttattttttcttttcttttccgaatcggattttgtttttaatatttcaaaaaaaaaaaaaaaaaaaaaa
aaaaaaaaaaaaaaaaaaaaaaagcggccgctcgagcgtcgaggggaattaattcttgaagacgaaagggccaggtggcactt
ttcggggaaatgtgcgcggaacccctatttgtttatttttctaaatacattcaaatatgtatccgctcatgagacaataaccctgataaat
gcttcaataatattgaaaaaggaagagtatgagtattcaacatttccgtgtcgcccttattcccttttttgcggcattttgccttcctgttttt
gctcacccagaaacgctggtgaaagtaaaagatgctgaagatcagttgggtgcacgagtgggttacatcgaactggatctcaaca
gcggtaagatccttgagagttttcgccccgaagaacgttttccaatgatgagcacttttaaagttctgctatgtggcgcggtattatcc
cgtgttgacgccgggcaagagcaactcggtcgccgcatacactattctcagaatgacttggttgagtactcaccagtcacagaaaa
gcatcttacggatggcatgacagtaagagaattatgcagtgctgccataaccatgagtgataacactgcggccaacttacttctgac
aacgatcggaggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaactattaactggc
gaactacttactctagcttcccggcaacaattaatagactggatggaggcggataaagttgcaggaccacttctgcgctcggccctt
ccggctggctggtttattgctgataaatctggagccggtgagcgtgggtctcgcggtatcattgcagcactggggccagatggtaa
gccctcccgtatcgtagttatctacacgacggggagtcaggcaactatggatgaacgaaatagacagatcgctgagataggtgcc
tcactgattaagcattggtaactgtcagaccaagtttactcatatatactttagattgatttaaaacttcatttttaatttaaaaggatctag
gtgaagatcctttttgataatctcatgaccaaaatcccttaacgtgagttttcgttccactgagcgtcagaccccgtagaaaagatcaa
aggatcttcttgagatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtccttctagtgtagccgtagtt
aggccaccacttcaagaactctgtagcaccgcctacatacctcgctctgctaatcctgttaccagtggctgctgccagtggcgataa
gtcgtgtcttaccgggttggactcaagacgatagttaccggataaggcgcagcggtcgggctgaacggggggttcgtgcacaca
gcccagcttggagcgaacgacctacaccgaactgagatacctacagcgtgagcattgagaaagcgccacgcttcccgaaggga
gaaaggcggacaggtatccggtaagcggcagggtcggaacaggagagcgcacgagggagcttccagggggaaacgcctgg
tatctttatagtcctgtcgggtttcgccacctctgacttgagcgtcgatttttgtgatgctcgtcaggggggcggagcctatggaaaaa
cgccagcaacgcgagctcgacgcgttaatacgactcactatag
plasmid pT7-VEE-SEAP-V2
SEQ ID NO: 5
ataggcggcgcatgagagaagcccagaccaattacctacccaaaatggagaaagttcacgttgacatcgaggaag
acagcccattcctcagagctttgcagcggagcttcccgcagtttgaggtagaagccaagcaggtcactgataatgaccatgctaat
gccagagcgttttcgcatctggcttcaaaactgatcgaaacggaggtggacccatccgacacgatccttgacattggaagtgcgc
ccgcccgcagaatgtattctaagcacaagtatcattgtatctgtccgatgagatgtgcggaagatccggacagattgtataagtatg
caactaagctgaagaaaaactgtaaggaaataactgataaggaattggacaagaaaatgaaggagctggccgccgtcatgagc
gaccctgacctggaaactgagactatgtgcctccacgacgacgagtcgtgtcgctacgaagggcaagtcgctgtttaccaggatg
tatacgcggttgacggaccgacaagtctctatcaccaagccaataagggagttagagtcgcctactggataggctttgacaccacc
ccttttatgtttaagaacttggctggagcatatccatcatactctaccaactgggccgacgaaaccgtgttaacggctcgtaacatag
gcctatgcagctctgacgttatggagcggtcacgtagagggatgtccattcttagaaagaagtatttgaaaccatccaacaatgttct
attctctgttggctcgaccatctaccacgagaagagggacttactgaggagctggcacctgccgtctgtatttcacttacgtggcaa
gcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggt
ctcttttcccgtgtgcacgtatgtgccagctacattgtgtgaccaaatgactggcatactggcaacagatgtcagtgcggacgacgc
gcaaaaactgctggttgggctcaaccagcgtatagtcgtcaacggtcgcacccagagaaacaccaataccatgaaaaattaccttt
tgcccgtagtggcccaggcatttgctaggtgggcaaaggaatataaggaagatcaagaagatgaaaggccactaggactacga
gatagacagttagtcatggggtgttgttgggcttttagaaggcacaagataacatctatttataagcgcccggatacccaaaccatc
atcaaagtgaacagcgatttccactcattcgtgctgcccaggataggcagtaacacattggagatcgggctgagaacaagaatca
ggaaaatgttagaggagcacaaggagccgtcacctctcattaccgccgaggacgtacaagaagctaagtgcgcagccgatgag
gctaaggaggtgcgtgaagccgaggagttgcgcgcagctctaccacctttggcagctgatgttgaggagcccactctggaggca
gacgtcgacttgatgttacaagaggctggggccggctcagtggagacacctcgtggcttgataaaggttaccagctacgatggcg
aggacaagatcggctcttacgctgtgctttctccgcaggctgtactcaagagtgaaaaattatcttgcatccaccctctcgctgaaca
agtcatagtgataacacactctggccgaaaagggcgttatgccgtggaaccataccatggtaaagtagtggtgccagagggacat
gcaatacccgtccaggactttcaagctctgagtgaaagtgccaccattgtgtacaacgaacgtgagttcgtaaacaggtacctgca
ccatattgccacacatggaggagcgctgaacactgatgaagaatattacaaaactgtcaagcccagcgagcacgacggcgaata
cctgtacgacatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtatggcgtgcca
ggatcaggcaagtctggcatcattaaaagcgcagtcaccaaaaaagatctagtggtgagcgccaagaaagaaaactgtgcagaa
attataagggacgtcaagaaaatgaaagggctggacgtcaatgccagaactgtggactcagtgctcttgaatggatgcaaacacc
ccgtagagaccctgtatattgacgaagcttttgcttgtcatgcaggtactctcagagcgctcatagccattataagacctaaaaaggc
agtgctctgcggggatcccaaacagtgcggtttttttaacatgatgtgcctgaaagtgcattttaaccacgagatttgcacacaagtct
tccacaaaagcatctctcgccgttgcactaaatctgtgacttcggtcgtctcaaccttgttttacgacaaaaaaatgagaacgacgaa
tccgaaagagactaagattgtgattgacactaccggcagtaccaaacctaagcaggacgatctcattctcacttgtttcagagggtg
ggtgaagcagttgcaaatagattacaaaggcaacgaaataatgacggcagctgcctctcaagggctgacccgtaaaggtgtgtat
gccgttcggtacaaggtgaatgaaaatcctctgtacgcacccacctcagaacatgtgaacgtcctactgacccgcacggaggacc
gcatcgtgtggaaaacactagccggcgacccatggataaaaacactgactgccaagtaccctgggaatttcactgccacgataga
ggagtggcaagcagagcatgatgccatcatgaggcacatcttggagagaccggaccctaccgacgtcttccagaataaggcaa
acgtgtgttgggccaaggctttagtgccggtgctgaagaccgctggcatagacatgaccactgaacaatggaacactgtggattat
tttgaaacggacaaagctcactcagcagagatagtattgaaccaactatgcgtgaggttctttggactcgatctggactccggtctat
tttctgcacccactgttccgttatccattaggaataatcactgggataactccccgtcgcctaacatgtacgggctgaataaagaagt
ggtccgtcagctctctcgcaggtacccacaactgcctcgggcagttgccactggaagagtctatgacatgaacactggtacactg
cgcaattatgatccgcgcataaacctagtacctgtaaacagaagactgcctcatgctttagtcctccaccataatgaacacccacag
agtgacttttcttcattcgtcagcaaattgaagggcagaactgtcctggtggtcggggaaaagttgtccgtcccaggcaaaatggtt
gactggttgtcagaccggcctgaggctaccttcagagctcggctggatttaggcatcccaggtgatgtgcccaaatatgacataat
atttgttaatgtgaggaccccatataaataccatcactatcagcagtgtgaagaccatgccattaagcttagcatgttgaccaagaaa
gcttgtctgcatctgaatcccggcggaacctgtgtcagcataggttatggttacgctgacagggccagcgaaagcatcattggtgc
tatagcgcggcagttcaagttttcccgggtatgcaaaccgaaatcctcacttgaagagacggaagttctgtttgtattcattgggtac
gatcgcaaggcccgtacgcacaatccttacaagctttcatcaaccttgaccaacatttatacaggttccagactccacgaagccgg
atgtgcaccctcatatcatgtggtgcgaggggatattgccacggccaccgaaggagtgattataaatgctgctaacagcaaagga
caacctggcggaggggtgtgcggagcgctgtataagaaattcccggaaagcttcgatttacagccgatcgaagtaggaaaagcg
cgactggtcaaaggtgcagctaaacatatcattcatgccgtaggaccaaacttcaacaaagtttcggaggttgaaggtgacaaaca
gttggcagaggcttatgagtccatcgctaagattgtcaacgataacaattacaagtcagtagcgattccactgttgtccaccggcatc
ttttccgggaacaaagatcgactaacccaatcattgaaccatttgctgacagctttagacaccactgatgcagatgtagccatatact
gcagggacaagaaatgggaaatgactctcaaggaagcagtggctaggagagaagcagtggaggagatatgcatatccgacga
ctcttcagtgacagaacctgatgcagagctggtgagggtgcatccgaagagttctttggctggaaggaagggctacagcacaag
cgatggcaaaactttctcatatttggaagggaccaagtttcaccaggcggccaaggatatagcagaaattaatgccatgtggcccg
ttgcaacggaggccaatgagcaggtatgcatgtatatcctcggagaaagcatgagcagtattaggtcgaaatgccccgtcgaaga
tcacgtccagaacaaattactgtgtgctcatcctttccattgccgaagtatagaatcactggtgtgcagaagatccaatgctcccagc
tcacgtccagaacaaattactgtgtgctcatcctttccattgccgaagtatagaatcactggtgtgcagaagatccaatgctcccagc
ctatattgttctcaccgaaagtgcctgcgtatattcatccaaggaagtatctcgtggaaacaccaccggtagacgagactccggag
ccatcggcagagaaccaatccacagaggggacacctgaacaaccaccacttataaccgaggatgagaccaggactagaacgc
ctgagccgatcatcatcgaagaggaagaagaggatagcataagtttgctgtcagatggcccgacccaccaggtgctgcaagtcg
aggcagacattcacgggccgccctctgtatctagctcatcctggtccattcctcatgcatccgactttgatgtggacagtttatccata
cttgacaccctggagggagctagcgtgaccagcggggcaacgtcagccgagactaactcttacttcgcaaagagtatggagtttc
tggcgcgaccggtgcctgcgcctcgaacagtattcaggaaccctccacatcccgctccgcgcacaagaacaccgtcacttgcac
ccagcagggcctgctcgagaaccagcctagtttccaccccgccaggcgtgaatagggtgatcactagagaggagctcgaggcg
cttaccccgtcacgcactcctagcaggtcggtctcgagaaccagcctggtctccaacccgccaggcgtaaatagggtgattacaa
gagaggagtttgaggcgttcgtagcacaacaacaatgacggtttgatgcgggtgcatacatcttttcctccgacaccggtcaaggg
catttacaacaaaaatcagtaaggcaaacggtgctatccgaagtggtgttggagaggaccgaattggagatttcgtatgccccgcg
cctcgaccaagaaaaagaagaattactacgcaagaaattacagttaaatcccacacctgctaacagaagcagataccagtccagg
aaggtggagaacatgaaagccataacagctagacgtattctgcaaggcctagggcattatttgaaggcagaaggaaaagtggag
tgctaccgaaccctgcatcctgttcctttgtattcatctagtgtgaaccgtgccttttcaagccccaaggtcgcagtggaagcctgtaa
cgccatgttgaaagagaactttccgactgtggcttcttactgtattattccagagtacgatgcctatttggacatggttgacggagcttc
atgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgaga
gaattgcccgtattggattcggcggcctttaatgtggaatgcttcaagaaatatgcgtgtaataatgaatattgggaaacgtttaaaga
aaaccccatcaggcttactgaagaaaacgtggtaaattacattaccaaattaaaaggaccaaaagctgctgctctttttgcgaagac
acataatttgaatatgttgcaggacataccaatggacaggtttgtaatggacttaaagagagacgtgaaagtgactccaggaacaa
aacatactgaagaacggcccaaggtacaggtgatccaggctgccgatccgctagcaacagcgtatctgtgcggaatccaccga
gagctggttaggagattaaatgcggtcctgcttccgaacattcatacactgtttgatatgtcggctgaagactttgacgctattatagc
cgagcacttccagcctggggattgtgttctggaaactgacatcgcgtcgtttgataaaagtgaggacgacgccatggctctgaccg
cgttaatgattctggaagacttaggtgtggacgcagagctgttgacgctgattgaggcggctttcggcgaaatttcatcaatacattt
gcccactaaaactaaatttaaattcggagccatgatgaaatctggaatgttcctcacactgtttgtgaacacagtcattaacattgtaat
cgcaagcagagtgttgagagaacggctaaccggatcaccatgtgcagcattcattggagatgacaatatcgtgaaaggagtcaa
atcggacaaattaatggcagacaggtgcgccacctggttgaatatggaagtcaagattatagatgctgtggtgggcgagaaagcg
ccttatttctgtggagggtttattttgtgtgactccgtgaccggcacagcgtgccgtgtggcagaccccctaaaaaggctgtttaagc
ttggcaaacctctggcagcagacgatgaacatgatgatgacaggagaagggcattgcatgaagagtcaacacgctggaaccga
gtgggtattctttcagagctgtgcaaggcagtagaatcaaggtatgaaaccgtaggaacttccatcatagttatggccatgactactc
tagctagcagtgttaaatcattcagctacctgagaggggcccctataactctctacggctaacctgaatggactacgacatagtcta
gtccgccaagatgctcctcttgctccttctcttgggactccgattgcagctctctttgggtataattcctgtcgaagaagagaatcccg
acttctggaatcgcgaagcggcagaagcattgggtgcggcaaaaaagctccaaccggcacagactgcggccaagaatctcata
attttcttgggtgatggcatgggcgtttccactgtgactgcggcacgaatcctgaagggtcaaaagaaggataaactgggtccaga
gatcccgctggctatggataggtttccatacgttgcgctctctaagacctataacgtcgataagcatgtaccagactccggagcgac
ggccactgcttacctttgtggagttaaaggtaatttccaaacaataggactgagcgcagctgcaagattcaaccaatgcaacacga
caagagggaatgaggtgatttcagtcatgaatcgagcaaagaaagctgggaaatccgttggcgtggtcaccactacaagagtgc
agcatgcatctccagcaggaacttacgcccacactgtaaacagaaactggtatagtgacgccgatgttcccgcctctgcgaggca
agagggttgtcaagacatcgctacccagctcataagcaacatggacattgatgtaatactgggtgggggtcggaaatacatgttcc
ggatgggcactcccgatccggagtacccggatgactattcccaaggaggtaccagattggacgggaaaaatcttgtccaagaat
ggctcgccaagcggcagggggcaaggtacgtgtggaacaggacagaactgatgcaggcaagcttggatccaagcgtaacgc
atcttatgggtctttttgaacccggtgatatgaaatacgaaatacatcgcgactcaacactggacccgtctctcatggagatgactga
agctgccttgaggttgttgagtcggaaccctaggggctttttcttgttcgtagagggcgggcgaattgaccacggtcatcacgaatc
tcgagcgtaccgggcgctcacagaaaccatcatgtttgacgatgctatcgaacgagcgggtcagcttacctctgaagaagatacg
ctctctcttgtcaccgcggaccatagccatgttttttccttcggtggttatccgttgcgaggttccagcatattcggcctcgcgccagg
gaaagcccgcgaccgcaaagcttatacggtgctgctttacggaaacggccctggttacgtccttaaagacggtgcgagacctga
cgtgacggaatctgaatccggttctcccgaatatagacaacagagtgctgttccgctggatgaagagactcatgcgggagaagat
gtagcggtttttgctagggggccgcaagcacaccttgttcatggcgttcaggagcaaactttcatagcccatgtaatggcatttgctg
cgtgtctcgagccgtataccgcttgcgatctcgctccgccggcgggtacaaccgatgctgcccacccggggtactcaagagtag
gggcagcagggcgatttgaacaaacttgataataaccgcggtgtcaaaaaccgcgtggacgtggttaacatccctgctgggagg
atcagccgtaattattataattggcttggtgctggctactattgtggccatgtacgtgctgaccaaccagaaacataattgaatacagc
agcaattggcaagctgcttacatagaactcgcggcgattggcatgccgccttaaaatttttattttattttttcttttcttttccgaatcgga
ttttgtttttaatatttcaaaaaaaaaaaaaaaaaaaaaaaaaaaaaaaaaaaaaaaaaaaaagcggccgcgagcttggctcgagc
ctcgagcatggtcatagctgtttcctgtgtgaaattgttatccgctcacaattccacacaacatacgagccggaagcataaagtgtaa
agcctggggtgcctaatgagtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcctcgctcactgactcgctgc
gctcggtcgttcggctgcggcgagcggtatcagctcactcaaaggcggtaatacggttatccacagaatcaggggataacgcag
gaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccc
cctggaagctccctcgtgcgctctcctgttccgaccctgccgcttaccggatacctgtccgcctttctcccttcgggaagcgtggcg
ctttctcatagctcacgctgtaggtatctcagttcggtgtaggtcgttcgctccaagctgggctgtgtgcacgaaccccccgttcagc
ccgaccgctgcgccttatccggtaactatcgtcttgagtccaacccggtaagacacgacttatcgccactggcagcagccactggt
aacaggattagcagagcgaggtatgtaggcggtgctacagagttcttgaagtggtggcctaactacggctacactagaagaaca
gtatttggtatctgcgctctgctgaagccagttaccttcggaaaaagagttggtagctcttgatccggcaaacaaaccaccgctggt
agcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaat
gaagttttaaatcaatctaaagtatatatgagtaaacttggtctgacagttagaaaaactcatcgagcatcaaatgaaactgcaatttat
tcatatcaggattatcaataccatatttttgaaaaagccgtttctgtaatgaaggagaaaactcaccgaggcagttccataggatggc
aagatcctggtatcggtctgcgattccgactcgtccaacatcaatacaacctattaatttcccctcgtcaaaaataaggttatcaagtg
agaaatcaccatgagtgacgactgaatccggtgagaatggcaaaagtttatgcatttctttccagacttgttcaacaggccagccatt
acgctcgtcatcaaaatcactcgcatcaaccaaaccgttattcattcgtgattgcgcctgagcgagacgaaatacgcgatcgctgtt
aaaaggacaattacaaacaggaatcgaatgcaaccggcgcaggaacactgccagcgcatcaacaatattttcacctgaatcagg
atattcttctaatacctggaatgctgttttcccagggatcgcagtggtgagtaaccatgcatcatcaggagtacggataaaatgcttga
tggtcggaagaggcataaattccgtcagccagtttagtctgaccatctcatctgtaacatcattggcaacgctacctttgccatgtttc
agaaacaactctggcgcatcgggcttcccatacaatcgatagattgtcgcacctgattgcccgacattatcgcgagcccatttatac
ccatataaatcagcatccatgttggaatttaatcgcggcctagagcaagacgtttcccgttgaatatggctcatactcttcctttttcaat
attattgaagcatttatcagggttattgtctcatgagcggatacatatttgaatgtatttagaaaaataaacaaataggggttccgcgca
catttccccgaaaagtgccacctgacgtctaagaaaccattattatcatgacattaacctataaaaataggcgtatcacgaggccctt
tcgtctcgcgcgtttcggtgatgacggtgaaaacctctgacacatgcagctcccggagacggtcacagcttgtctgtaagcggatg
ccgggagcagacaagcccgtcagggcgcgtcagcgggtgttggcgggtgtcggggctggcttaactatgcggcatcagagca
gattgtactgagagtgcaccatatgcggtgtgaaataccgcacagatgcgtaaggagaaaataccgcatcaggcgccattcgcca
ttcaggctgcgcaactgttgggaagggcgatcggtgcgggcctcttcgctattacgccagctggcgaaagggggatgtgctgca
aggcgattaagttgggtaacgccagggttttcccagtcacgacgttgtaaaacgacggccagtgaattgacgcgttaatacgactc
actatag
plasmid p506 VEE-SARS-CoV-2 CO S protein N9-D614G-2P-Kan
SEQ ID NO: 6
ataggcggcgcatgagagaagcccagaccaattacctacccaaaatggagaaagttcacgttgacatcgaggaag
acagcccattcctcagagctttgcagcggagcttcccgcagtttgaggtagaagccaagcaggtcactgataatgaccatgctaat
gccagagcgttttcgcatctggcttcaaaactgatcgaaacggaggtggacccatccgacacgatccttgacattggaagtgcgc
ccgcccgcagaatgtattctaagcacaagtatcattgtatctgtccgatgagatgtgcggaagatccggacagattgtataagtatg
caactaagctgaagaaaaactgtaaggaaataactgataaggaattggacaagaaaatgaaggagctggccgccgtcatgagc
gaccctgacctggaaactgagactatgtgcctccacgacgacgagtcgtgtcgctacgaagggcaagtcgctgtttaccaggatg
tatacgcggttgacggaccgacaagtctctatcaccaagccaataagggagttagagtcgcctactggataggctttgacaccacc
ccttttatgtttaagaacttggctggagcatatccatcatactctaccaactgggccgacgaaaccgtgttaacggctcgtaacatag
gcctatgcagctctgacgttatggagcggtcacgtagagggatgtccattcttagaaagaagtatttgaaaccatccaacaatgttct
attctctgttggctcgaccatctaccacgagaagagggacttactgaggagctggcacctgccgtctgtatttcacttacgtggcaa
gcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggt
ctcttttcccgtgtgcacgtatgtgccagctacattgtgtgaccaaatgactggcatactggcaacagatgtcagtgcggacgacgc
gcaaaaactgctggttgggctcaaccagcgtatagtcgtcaacggtcgcacccagagaaacaccaataccatgaaaaattaccttt
tgcccgtagtggcccaggcatttgctaggtgggcaaaggaatataaggaagatcaagaagatgaaaggccactaggactacga
gatagacagttagtcatggggtgttgttgggcttttagaaggcacaagataacatctatttataagcgcccggatacccaaaccatc
atcaaagtgaacagcgatttccactcattcgtgctgcccaggataggcagtaacacattggagatcgggctgagaacaagaatca
ggaaaatgttagaggagcacaaggagccgtcacctctcattaccgccgaggacgtacaagaagctaagtgcgcagccgatgag
gctaaggaggtgcgtgaagccgaggagttgcgcgcagctctaccacctttggcagctgatgttgaggagcccactctggaggca
gacgtcgacttgatgttacaagaggctggggccggctcagtggagacacctcgtggcttgataaaggttaccagctacgatggcg
aggacaagatcggctcttacgctgtgctttctccgcaggctgtactcaagagtgaaaaattatcttgcatccaccctctcgctgaaca
agtcatagtgataacacactctggccgaaaagggcgttatgccgtggaaccataccatggtaaagtagtggtgccagagggacat
gcaatacccgtccaggactttcaagctctgagtgaaagtgccaccattgtgtacaacgaacgtgagttcgtaaacaggtacctgca
ccatattgccacacatggaggagcgctgaacactgatgaagaatattacaaaactgtcaagcccagcgagcacgacggcgaata
cctgtacgacatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtatggcgtgcca
ggatcaggcaagtctggcatcattaaaagcgcagtcaccaaaaaagatctagtggtgagcgccaagaaagaaaactgtgcagaa
attataagggacgtcaagaaaatgaaagggctggacgtcaatgccagaactgtggactcagtgctcttgaatggatgcaaacacc
ccgtagagaccctgtatattgacgaagcttttgcttgtcatgcaggtactctcagagcgctcatagccattataagacctaaaaaggc
agtgctctgcggggatcccaaacagtgcggtttttttaacatgatgtgcctgaaagtgcattttaaccacgagatttgcacacaagtct
tccacaaaagcatctctcgccgttgcactaaatctgtgacttcggtcgtctcaaccttgttttacgacaaaaaaatgagaacgacgaa
tccgaaagagactaagattgtgattgacactaccggcagtaccaaacctaagcaggacgatctcattctcacttgtttcagagggtg
ggtgaagcagttgcaaatagattacaaaggcaacgaaataatgacggcagctgcctctcaagggctgacccgtaaaggtgtgtat
gccgttcggtacaaggtgaatgaaaatcctctgtacgcacccacctcagaacatgtgaacgtcctactgacccgcacggaggacc
gcatcgtgtggaaaacactagccggcgacccatggataaaaacactgactgccaagtaccctgggaatttcactgccacgataga
ggagtggcaagcagagcatgatgccatcatgaggcacatcttggagagaccggaccctaccgacgtcttccagaataaggcaa
acgtgtgttgggccaaggctttagtgccggtgctgaagaccgctggcatagacatgaccactgaacaatggaacactgtggattat
tttgaaacggacaaagctcactcagcagagatagtattgaaccaactatgcgtgaggttctttggactcgatctggactccggtctat
tttctgcacccactgttccgttatccattaggaataatcactgggataactccccgtcgcctaacatgtacgggctgaataaagaagt
ggtccgtcagctctctcgcaggtacccacaactgcctcgggcagttgccactggaagagtctatgacatgaacactggtacactg
cgcaattatgatccgcgcataaacctagtacctgtaaacagaagactgcctcatgctttagtcctccaccataatgaacacccacag
agtgacttttcttcattcgtcagcaaattgaagggcagaactgtcctggtggtcggggaaaagttgtccgtcccaggcaaaatggtt
gactggttgtcagaccggcctgaggctaccttcagagctcggctggatttaggcatcccaggtgatgtgcccaaatatgacataat
atttgttaatgtgaggaccccatataaataccatcactatcagcagtgtgaagaccatgccattaagcttagcatgttgaccaagaaa
gcttgtctgcatctgaatcccggcggaacctgtgtcagcataggttatggttacgctgacagggccagcgaaagcatcattggtgc
tatagcgcggcagttcaagttttcccgggtatgcaaaccgaaatcctcacttgaagagacggaagttctgtttgtattcattgggtac
gatcgcaaggcccgtacgcacaatccttacaagctttcatcaaccttgaccaacatttatacaggttccagactccacgaagccgg
atgtgcaccctcatatcatgtggtgcgaggggatattgccacggccaccgaaggagtgattataaatgctgctaacagcaaagga
caacctggcggaggggtgtgcggagcgctgtataagaaattcccggaaagcttcgatttacagccgatcgaagtaggaaaagcg
cgactggtcaaaggtgcagctaaacatatcattcatgccgtaggaccaaacttcaacaaagtttcggaggttgaaggtgacaaaca
gttggcagaggcttatgagtccatcgctaagattgtcaacgataacaattacaagtcagtagcgattccactgttgtccaccggcatc
ttttccgggaacaaagatcgactaacccaatcattgaaccatttgctgacagctttagacaccactgatgcagatgtagccatatact
gcagggacaagaaatgggaaatgactctcaaggaagcagtggctaggagagaagcagtggaggagatatgcatatccgacga
ctcttcagtgacagaacctgatgcagagctggtgagggtgcatccgaagagttctttggctggaaggaagggctacagcacaag
cgatggcaaaactttctcatatttggaagggaccaagtttcaccaggcggccaaggatatagcagaaattaatgccatgtggcccg
ttgcaacggaggccaatgagcaggtatgcatgtatatcctcggagaaagcatgagcagtattaggtcgaaatgccccgtcgaaga
gtcggaagcctccacaccacctagcacgctgccttgcttgtgcatccatgccatgactccagaaagagtacagcgcctaaaagcc
tcacgtccagaacaaattactgtgtgctcatcctttccattgccgaagtatagaatcactggtgtgcagaagatccaatgctcccagc
ctatattgttctcaccgaaagtgcctgcgtatattcatccaaggaagtatctcgtggaaacaccaccggtagacgagactccggag
ccatcggcagagaaccaatccacagaggggacacctgaacaaccaccacttataaccgaggatgagaccaggactagaacgc
ctgagccgatcatcatcgaagaggaagaagaggatagcataagtttgctgtcagatggcccgacccaccaggtgctgcaagtcg
aggcagacattcacgggccgccctctgtatctagctcatcctggtccattcctcatgcatccgactttgatgtggacagtttatccata
cttgacaccctggagggagctagcgtgaccagcggggcaacgtcagccgagactaactcttacttcgcaaagagtatggagtttc
tggcgcgaccggtgcctgcgcctcgaacagtattcaggaaccctccacatcccgctccgcgcacaagaacaccgtcacttgcac
ccagcagggcctgctcgagaaccagcctagtttccaccccgccaggcgtgaatagggtgatcactagagaggagctcgaggcg
cttaccccgtcacgcactcctagcaggtcggtctcgagaaccagcctggtctccaacccgccaggcgtaaatagggtgattacaa
gagaggagtttgaggcgttcgtagcacaacaacaatgacggtttgatgcgggtgcatacatcttttcctccgacaccggtcaaggg
catttacaacaaaaatcagtaaggcaaacggtgctatccgaagtggtgttggagaggaccgaattggagatttcgtatgccccgcg
cctcgaccaagaaaaagaagaattactacgcaagaaattacagttaaatcccacacctgctaacagaagcagataccagtccagg
aaggtggagaacatgaaagccataacagctagacgtattctgcaaggcctagggcattatttgaaggcagaaggaaaagtggag
tgctaccgaaccctgcatcctgttcctttgtattcatctagtgtgaaccgtgccttttcaagccccaaggtcgcagtggaagcctgtaa
cgccatgttgaaagagaactttccgactgtggcttcttactgtattattccagagtacgatgcctatttggacatggttgacggagcttc
atgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgaga
gaattgcccgtattggattcggcggcctttaatgtggaatgcttcaagaaatatgcgtgtaataatgaatattgggaaacgtttaaaga
aaaccccatcaggcttactgaagaaaacgtggtaaattacattaccaaattaaaaggaccaaaagctgctgctctttttgcgaagac
acataatttgaatatgttgcaggacataccaatggacaggtttgtaatggacttaaagagagacgtgaaagtgactccaggaacaa
aacatactgaagaacggcccaaggtacaggtgatccaggctgccgatccgctagcaacagcgtatctgtgcggaatccaccga
gagctggttaggagattaaatgcggtcctgcttccgaacattcatacactgtttgatatgtcggctgaagactttgacgctattatagc
cgagcacttccagcctggggattgtgttctggaaactgacatcgcgtcgtttgataaaagtgaggacgacgccatggctctgaccg
cgttaatgattctggaagacttaggtgtggacgcagagctgttgacgctgattgaggcggctttcggcgaaatttcatcaatacattt
gcccactaaaactaaatttaaattcggagccatgatgaaatctggaatgttcctcacactgtttgtgaacacagtcattaacattgtaat
cgcaagcagagtgttgagagaacggctaaccggatcaccatgtgcagcattcattggagatgacaatatcgtgaaaggagtcaa
atcggacaaattaatggcagacaggtgcgccacctggttgaatatggaagtcaagattatagatgctgtggtgggcgagaaagcg
ccttatttctgtggagggtttattttgtgtgactccgtgaccggcacagcgtgccgtgtggcagaccccctaaaaaggctgtttaagc
ttggcaaacctctggcagcagacgatgaacatgatgatgacaggagaagggcattgcatgaagagtcaacacgctggaaccga
gtgggtattctttcagagctgtgcaaggcagtagaatcaaggtatgaaaccgtaggaacttccatcatagttatggccatgactactc
tagctagcagtgttaaatcattcagctacctgagaggggcccctataactctctacggctaacctgaatggactacgacatagtcta
gtccgccaagATGTTCCTGCTGACCACCAAACGCACCATGTTCGTGTTCCTGGTTCT
TCTGCCTCTGGTGTCTAGCCAGTGTGTGAATCTGACCACAAGGACCCAACTTC
CTCCTGCCTACACAAACAGCTTCACCAGAGGCGTGTACTACCCTGATAAGGTG
TTCCGGTCCTCAGTGTTGCATAGCACGCAGGACCTCTTTCTGCCCTTCTTCAGC
AACGTGACCTGGTTCCACGCCATCCATGTGTCTGGCACCAATGGCACCAAGAG
ATTCGACAATCCCGTTCTGCCCTTCAATGATGGCGTGTACTTTGCCAGCACCGA
GAAGAGCAACATCATCCGGGGATGGATTTTTGGTACTACTTTAGATAGCAAGA
CACAGTCTCTGCTGATCGTGAACAATGCCACCAACGTGGTGATTAAGGTGTGC
GAGTTCCAGTTCTGCAACGACCCCTTTCTGGGCGTGTATTACCACAAGAACAA
CAAGTCCTGGATGGAGAGCGAGTTCCGGGTGTATAGTTCAGCAAACAATTGCA
CATTCGAATATGTTTCTCAGCCTTTCCTGATGGACCTGGAGGGCAAACAGGGC
AATTTTAAAAACTTACGGGAGTTTGTGTTCAAGAACATCGACGGCTATTTTAA
GATCTACTCAAAACACACTCCTATAAACCTGGTGAGGGACCTGCCTCAGGGCT
TCTCAGCCCTAGAGCCTCTCGTCGATCTCCCTATCGGCATCAACATCACCCGGT
TCCAGACCCTGTTAGCTCTGCACAGAAGCTATCTGACACCTGGCGATTCTTCTT
CTGGATGGACAGCTGGAGCTGCCGCCTATTATGTGGGCTATTTACAGCCTAGA
ACCTTCCTGTTGAAGTACAACGAGAATGGCACCATCACCGACGCTGTGGATTG
TGCTCTTGATCCTCTGTCTGAGACCAAGTGTACCCTGAAGAGCTTCACAGTGG
AGAAGGGCATCTACCAGACCAGCAACTTCAGAGTGCAGCCTACAGAGAGCAT
CGTGAGATTCCCCAACATCACCAACCTGTGCCCATTTGGCGAGGTGTTTAATG
CCACCAGATTCGCATCAGTGTACGCATGGAACAGAAAGAGGATCAGCAATTG
CGTGGCCGATTATAGCGTGTTGTACAATTCAGCTTCGTTTAGCACGTTCAAGTG
TTATGGCGTATCCCCTACCAAGCTGAATGACCTGTGCTTCACAAACGTCTACG
CTGACAGCTTCGTGATTAGAGGCGATGAGGTGAGACAGATTGCTCCTGGACAA
ACAGGCAAGATTGCCGACTACAACTACAAGCTGCCCGACGACTTTACCGGCTG
TGTGATTGCCTGGAATTCTAATAACCTTGATAGTAAAGTGGGAGGGAATTACA
ATTATCTCTACCGGCTTTTCCGGAAGAGCAACCTGAAGCCATTCGAGAGAGAT
ATCAGCACCGAGATCTATCAGGCTGGCAGCACACCCTGTAATGGAGTGGAGG
GCTTCAACTGCTACTTTCCTCTGCAAAGCTATGGCTTTCAACCCACAAACGGA
GTGGGATATCAGCCCTACAGAGTGGTTGTTCTGAGCTTCGAACTGCTGCATGC
TCCTGCTACAGTGTGTGGCCCTAAAAAGAGTACTAATCTGGTCAAAAATAAGT
GCGTGAACTTCAATTTCAATGGCCTGACCGGCACAGGAGTTCTGACAGAGAGC
AACAAAAAGTTCCTCCCTTTCCAGCAGTTTGGAAGGGATATCGCCGACACCAC
AGATGCCGTGAGAGATCCTCAAACACTGGAGATCCTGGACATTACCCCTTGCT
CTTTTGGAGGCGTGAGCGTGATCACACCTGGCACAAATACCAGCAATCAGGTG
GCTGTGCTGTATCAGGGAGTGAATTGCACCGAGGTTCCAGTGGCCATTCATGC
TGATCAACTGACCCCTACCTGGAGAGTGTACAGCACAGGCTCTAACGTGTTTC
AGACCAGAGCTGGATGTCTGATTGGAGCCGAACACGTGAACAACAGCTACGA
GTGCGATATCCCTATTGGAGCCGGCATTTGTGCCTCTTACCAGACACAGACCA
ATAGCCCCAGAAGAGCCAGATCTGTGGCTTCTCAGAGCATTATCGCCTACACC
ATGTCTCTGGGAGCCGAGAATTCTGTGGCCTACAGCAACAACTCTATCGCCAT
CCCTACCAACTTCACCATCAGCGTGACCACCGAGATTCTGCCTGTGAGCATGA
CAAAGACAAGCGTGGATTGCACCATGTACATCTGCGGCGATAGCACCGAGTG
CAGCAATCTGCTGTTACAGTACGGAAGTTTTTGTACCCAGCTGAATAGAGCCC
TGACAGGCATTGCCGTGGAACAGGACAAGAACACACAGGAGGTGTTTGCTCA
GGTGAAACAGATCTACAAGACTCCCCCTATAAAGGACTTTGGCGGCTTCAACT
TCAGCCAGATTCTGCCTGATCCTTCTAAGCCTAGCAAGCGGAGCTTCATCGAA
GACCTGCTGTTCAACAAGGTGACACTGGCCGATGCCGGCTTTATTAAGCAGTA
CGGCGATTGTCTGGGCGATATCGCTGCCAGAGATCTGATTTGCGCCCAGAAAT
TCAATGGTCTAACAGTGCTTCCTCCTCTGCTGACAGATGAGATGATTGCCCAG
TACACAAGCGCTCTGTTAGCCGGCACAATTACATCTGGATGGACATTTGGAGC
TGGAGCTGCTCTGCAAATTCCTTTTGCCATGCAGATGGCCTACAGATTCAATG
GGATCGGAGTGACCCAGAACGTGCTGTACGAGAACCAGAAGCTCATAGCCAA
CCAGTTCAATTCTGCCATCGGCAAGATCCAGGACAGCCTGAGCTCTACAGCTT
CTGCTCTGGGCAAACTGCAGGATGTTGTGAATCAGAATGCGCAGGCTTTAAAC
ACTCTGGTGAAACAGCTGAGCAGCAATTTTGGCGCCATCAGCTCTGTGCTTAA
TGACATCCTGAGCAGGCTGGACCCTCCTGAAGCTGAAGTGCAAATCGACCGGC
TCATCACCGGGCGCCTGCAGTCTCTGCAGACATACGTCACTCAGCAACTGATC
AGAGCTGCCGAGATTCGCGCGAGTGCCAATCTGGCTGCCACCAAGATGTCTGA
GTGTGTTCTGGGGCAATCAAAGCGCGTGGATTTCTGCGGCAAGGGATATCACC
TGATGAGCTTCCCTCAGTCTGCTCCTCATGGAGTGGTGTTCCTGCATGTGACCT
ATGTGCCTGCTCAGGAGAAGAATTTCACAACAGCCCCTGCCATCTGCCACGAT
GGAAAAGCCCACTTTCCAAGAGAAGGCGTGTTCGTGTCTAATGGAACACACTG
GTTCGTGACCCAGCGGAACTTCTACGAACCCCAGATCATCACCACCGACAACA
CATTTGTGAGCGGCAATTGCGATGTGGTGATCGGCATCGTGAACAACACCGTG
TACGACCCTCTGCAACCTGAACTGGACAGCTTTAAGGAGGAGCTGGACAAGT
ACTTTAAGAACCATACGAGCCCTGACGTGGATCTGGGCGACATCAGTGGTATC
AATGCTAGCGTGGTGAATATCCAGAAGGAGATCGACCGGCTGAATGAAGTGG
CCAAGAACCTGAACGAAAGCCTGATCGACCTGCAAGAACTGGGCAAGTATGA
GCAGTACATCAAGTGGCCCTGGTACATCTGGCTGGGCTTTATTGCCGGACTGA
TCGCCATCGTTATGGTGACCATTATGCTGTGCTGCATGACCAGCTGCTGCTCTT
GTCTGAAGGGCTGTTGCTCTTGTGGCTCTTGCTGTAAGTTCGATGAGGACGATT
CCGAGCCTGTCCTCAAGGGGGTCAAACTCCACTACACCTGATGAccgcggtgtcaaaa
accgcgtggacgtggttaacatccctgctgggaggatcagccgtaattattataattggcttggtgctggctactattgtggccatgt
acgtgctgaccaaccagaaacataattgaatacagcagcaattggcaagctgcttacatagaactcgcggcgattggcatgccgc
cttaaaatttttattttattttttcttttcttttccgaatcggattttgtttttaatatttcaaaaaaaaaaaaaaaaaaaaaaaaaaaaaaaaaa
aaaaaaaaaaagcggccgcgagcttggctcgagcctcgagcatggtcatagctgtttcctgtgtgaaattgttatccgctcacaatt
ccacacaacatacgagccggaagcataaagtgtaaagcctggggtgcctaatgagtgagctaactcacattaattgcgttgcgctc
actgcccgctttccagtcgggaaacctgtcgtgccagctgcattaatgaatcggccaacgcgcggggagaggcggtttgcgtatt
gggcgctcttccgcttcctcgctcactgactcgctgcgctcggtcgttcggctgcggcgagcggtatcagctcactcaaaggcggt
aatacggttatccacagaatcaggggataacgcaggaaagaacatgtgagcaaaaggccagcaaaaggccaggaaccgtaaa
aaggccgcgttgctggcgtttttccataggctccgcccccctgacgagcatcacaaaatcacaaaaatcgacgctcaagtcagag
gtggcgaaacccgacaggactataaagataccaggcgtttccccctggaagctccctcgtgcgctctcctgttccgaccctgccg
cttaccggatacctgtccgcctttctcccttcgggaagcgtggcgctttctcatagctcacgctgtaggtatctcagttcggtgtaggt
cgttcgctccaagctgggctgtgtgcacgaaccccccgttcagcccgaccgctgcgccttatccggtaactatcgtcttgagtcca
acccggtaagacacgacttatcgccactggcagcagccactggtaacaggattagcagagcgaggtatgtaggcggtgctaca
gagttcttgaagtggtggcctaactacggctacactagaagaacagtatttggtatctgcgctctgctgaagccagttaccttcggaa
aaagagttggtagctcttgatccggcaaacaaaccaccgctggtagcggtggtttttttgtttgcaagcagcagattacgcgcagaa
aaaaaggatctcaagaagatcctttgatcttttctacggggtctgacgctcagtggaacgaaaactcacgttaagggattttggtcat
gagattatcaaaaaggatcttcacctagatccttttaaattaaaaatgaagttttaaatcaatctaaagtatatatgagtaaacttggtct
gacagttagaaaaactcatcgagcatcaaatgaaactgcaatttattcatatcaggattatcaataccatatttttgaaaaagccgtttc
tgtaatgaaggagaaaactcaccgaggcagttccataggatggcaagatcctggtatcggtctgcgattccgactcgtccaacatc
aatacaacctattaatttcccctcgtcaaaaataaggttatcaagtgagaaatcaccatgagtgacgactgaatccggtgagaatgg
caaaagtttatgcatttctttccagacttgttcaacaggccagccattacgctcgtcatcaaaatcactcgcatcaaccaaaccgttatt
cattcgtgattgcgcctgagcgagacgaaatacgcgatcgctgttaaaaggacaattacaaacaggaatcgaatgcaaccggcg
caggaacactgccagcgcatcaacaatattttcacctgaatcaggatattcttctaatacctggaatgctgttttcccagggatcgca
gtggtgagtaaccatgcatcatcaggagtacggataaaatgcttgatggtcggaagaggcataaattccgtcagccagtttagtctg
accatctcatctgtaacatcattggcaacgctacctttgccatgtttcagaaacaactctggcgcatcgggcttcccatacaatcgata
gattgtcgcacctgattgcccgacattatcgcgagcccatttatacccatataaatcagcatccatgttggaatttaatcgcggcctag
agcaagacgtttcccgttgaatatggctcatactcttcctttttcaatattattgaagcatttatcagggttattgtctcatgagcggatac
atatttgaatgtatttagaaaaataaacaaataggggttccgcgcacatttccccgaaaagtgccacctgacgtctaagaaaccatta
ttatcatgacattaacctataaaaataggcgtatcacgaggccctttcgtctcgcgcgtttcggtgatgacggtgaaaacctctgaca
catgcagctcccggagacggtcacagcttgtctgtaagcggatgccgggagcagacaagcccgtcagggcgcgtcagcgggt
gttggcgggtgtcggggctggcttaactatgcggcatcagagcagattgtactgagagtgcaccatatgcggtgtgaaataccgc
acagatgcgtaaggagaaaataccgcatcaggcgccattcgccattcaggctgcgcaactgttgggaagggcgatcggtgcgg
gcctcttcgctattacgccagctggcgaaagggggatgtgctgcaaggcgattaagttgggtaacgccagggttttcccagtcac
gacgttgtaaaacgacggccagtgaattgacgcgttaatacgactcactatag
SARS-CoV-2 spike protein
SEQ ID NO: 7
atgttcctgctgaccaccaaacgcaccatgttcgtgttcctggttcttctgcctctggtgtctagccagtgtgtgaatctg
accacaaggacccaacttcctcctgcctacacaaacagcttcaccagaggcgtgtactaccctgataaggtgttccggtcctcagt
gttgcatagcacgcaggacctctttctgcccttcttcagcaacgtgacctggttccacgccatccatgtgtctggcaccaatggcac
caagagattcgacaatcccgttctgcccttcaatgatggcgtgtactttgccagcaccgagaagagcaacatcatccggggatgg
atttttggtactactttagatagcaagacacagtctctgctgatcgtgaacaatgccaccaacgtggtgattaaggtgtgcgagttcca
gttctgcaacgacccctttctgggcgtgtattaccacaagaacaacaagtcctggatggagagcgagttccgggtgtatagttcag
caaacaattgcacattcgaatatgtttctcagcctttcctgatggacctggagggcaaacagggcaattttaaaaacttacgggagtt
tgtgttcaagaacatcgacggctattttaagatctactcaaaacacactcctataaacctggtgagggacctgcctcagggcttctca
gccctagagcctctcgtcgatctccctatcggcatcaacatcacccggttccagaccctgttagctctgcacagaagctatctgaca
cctggcgattcttcttctggatggacagctggagctgccgcctattatgtgggctatttacagcctagaaccttcctgttgaagtacaa
cgagaatggcaccatcaccgacgctgtggattgtgctcttgatcctctgtctgagaccaagtgtaccctgaagagcttcacagtgg
agaagggcatctaccagaccagcaacttcagagtgcagcctacagagagcatcgtgagattccccaacatcaccaacctgtgcc
catttggcgaggtgtttaatgccaccagattcgcatcagtgtacgcatggaacagaaagaggatcagcaattgcgtggccgattat
agcgtgttgtacaattcagcttcgtttagcacgttcaagtgttatggcgtatcccctaccaagctgaatgacctgtgcttcacaaacgt
ctacgctgacagcttcgtgattagaggcgatgaggtgagacagattgctcctggacaaacaggcaagattgccgactacaactac
aagctgcccgacgactttaccggctgtgtgattgcctggaattctaataaccttgatagtaaagtgggagggaattacaattatctcta
ccggcttttccggaagagcaacctgaagccattcgagagagatatcagcaccgagatctatcaggctggcagcacaccctgtaat
ggagtggagggcttcaactgctactttcctctgcaaagctatggctttcaacccacaaacggagtgggatatcagccctacagagt
ggttgttctgagcttcgaactgctgcatgctcctgctacagtgtgtggccctaaaaagagtactaatctggtcaaaaataagtgcgtg
aacttcaatttcaatggcctgaccggcacaggagttctgacagagagcaacaaaaagttcctccctttccagcagtttggaaggga
tatcgccgacaccacagatgccgtgagagatcctcaaacactggagatcctggacattaccccttgctcttttggaggcgtgagcg
tgatcacacctggcacaaataccagcaatcaggtggctgtgctgtatcagggagtgaattgcaccgaggttccagtggccattcat
gctgatcaactgacccctacctggagagtgtacagcacaggctctaacgtgtttcagaccagagctggatgtctgattggagccga
acacgtgaacaacagctacgagtgcgatatccctattggagccggcatttgtgcctcttaccagacacagaccaatagccccaga
agagccagatctgtggcttctcagagcattatcgcctacaccatgtctctgggagccgagaattctgtggcctacagcaacaactct
atcgccatccctaccaacttcaccatcagcgtgaccaccgagattctgcctgtgagcatgacaaagacaagcgtggattgcaccat
gtacatctgcggcgatagcaccgagtgcagcaatctgctgttacagtacggaagtttttgtacccagctgaatagagccctgacag
gcattgccgtggaacaggacaagaacacacaggaggtgtttgctcaggtgaaacagatctacaagactccccctataaaggactt
tggcggcttcaacttcagccagattctgcctgatccttctaagcctagcaagcggagcttcatcgaagacctgctgttcaacaaggt
gacactggccgatgccggctttattaagcagtacggcgattgtctgggcgatatcgctgccagagatctgatttgcgcccagaaatt
caatggtctaacagtgcttcctcctctgctgacagatgagatgattgcccagtacacaagcgctctgttagccggcacaattacatct
ggatggacatttggagctggagctgctctgcaaattccttttgccatgcagatggcctacagattcaatgggatcggagtgaccca
gaacgtgctgtacgagaaccagaagctcatagccaaccagttcaattctgccatcggcaagatccaggacagcctgagctctaca
gcttctgctctgggcaaactgcaggatgttgtgaatcagaatgcgcaggctttaaacactctggtgaaacagctgagcagcaatttt
ggcgccatcagctctgtgcttaatgacatcctgagcaggctggaccctcctgaagctgaagtgcaaatcgaccggctcatcaccg
ggcgcctgcagtctctgcagacatacgtcactcagcaactgatcagagctgccgagattcgcgcgagtgccaatctggctgccac
caagatgtctgagtgtgttctggggcaatcaaagcgcgtggatttctgcggcaagggatatcacctgatgagcttccctcagtctgc
tcctcatggagtggtgttcctgcatgtgacctatgtgcctgctcaggagaagaatttcacaacagcccctgccatctgccacgatgg
aaaagcccactttccaagagaaggcgtgttcgtgtctaatggaacacactggttcgtgacccagcggaacttctacgaaccccag
atcatcaccaccgacaacacatttgtgagcggcaattgcgatgtggtgatcggcatcgtgaacaacaccgtgtacgaccctctgca
acctgaactggacagctttaaggaggagctggacaagtactttaagaaccatacgagccctgacgtggatctgggcgacatcagt
ggtatcaatgctagcgtggtgaatatccagaaggagatcgaccggctgaatgaagtggccaagaacctgaacgaaagcctgatc
gacctgcaagaactgggcaagtatgagcagtacatcaagtggccctggtacatctggctgggctttattgccggactgatcgccat
cgttatggtgaccattatgctgtgctgcatgaccagctgctgctcttgtctgaagggctgttgctcttgtggctcttgctgtaagttcgat
gaggacgattccgagcctgtcctcaagggggtcaaactccactacacctgatga
Nine N-terminal codons of SARS-CoV2 spike protein
SEQ ID NO: 8
MFLLTTKRT

### CONCLUSION

Although the subject matter has been described in language specific to features and/or methodological acts, it is to be understood that the subject matter defined in the appended claims is not necessarily limited to the specific features or acts described above. Rather, the specific features and acts are disclosed as example forms of implementing the claims.

Certain implementations are described herein, including the best mode known to the inventors for carrying out the invention. Of course, variations on these described implementations will become apparent to those of ordinary skill in the art upon reading the foregoing description. Skilled artisans will know how to employ such variations as appropriate, and the implementations disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

All references listed herein, including patent applications and patent publications are herein incorporated by reference in their entirety, as if each individual reference is specifically and individually indicated to be incorporated by reference.

1. A thermostable, lyophilized composition for delivery of a bioactive agent to a cell, the composition comprising:
a) nanostructured lipid carrier (NLC) particles comprising:
an oil core comprising a mixture of a liquid phase lipid and a solid phase lipid;
a cationic lipid;
a hydrophobic surfactant; and
a hydrophilic surfactant; and
b) a cake-forming excipient, wherein the composition is in the form of a cake and forms an oil-in-water emulsion upon reconstitution.

2. The composition of claim Implementation 1, further comprising:
c) the bioactive agent, wherein the bioactive agent comprises RNA.

3. The composition of claim Implementation 2, wherein the RNA comprises a replicon.

4. The composition of claim Implementation 2, wherein the RNA is self-amplifying RNA (saRNA).

5. The composition of claim Implementation 2, wherein the RNA is messenger RNA (mRNA).

6. The composition of any of claims Implementation 2 -Implementation 5, wherein the RNA encodes an antigen.

7. The composition of claim Implementation 6, wherein the antigen comprises the Zika pre-membrane (PrM) and envelope (E) proteins.

8. The composition of claim Implementation 6, wherein the antigen comprises the SARS-CoV-2 spike protein.

9. The composition of any of claims Implementation 2 -Implementation 8, wherein the bioactive agent is electrostatically complexed to the outer surface of the NLC particles.

10. The composition of any of claims Implementation 1 -Implementation 9, wherein the liquid phase lipid is metabolizable.

11. The composition of any of claims Implementation 1 -Implementation 10, wherein the liquid phase lipid is a vegetable oil, animal oil, or synthetically prepared oil.

12. The composition of any of claims Implementation 1 -Implementation 10, wherein the liquid phase lipid is capric/caprylic triglyceride, vitamin E, lauroyl polyoxylglyceride, monoacylglycerol, soy lecithin, squalene, synthetic squalene, squalene, or a combination thereof.

13. The composition of any of claims Implementation 1 -Implementation 10, wherein the liquid phase lipid is a naturally occurring or synthetic terpenoid.

14. The composition of any of claims Implementation 1 -Implementation 10, wherein the liquid phase lipid is squalene or synthetic squalene.

15. The composition of any of claims Implementation 1 -Implementation 14, wherein the solid phase lipid is a glycerolipid.

16. The composition of any of claims Implementation 1 -Implementation 14, wherein the solid phase lipid is a microcrystalline triglyceride.

17. The composition of claim Implementation 16, wherein the microcrystalline triglyceride is trimyristin.

18. The composition of any of claims Implementation 1 -Implementation 17, wherein the cationic lipid is 1,2-dioleoyloxy-3-(trimethylammonio)propane (DOTAP), 3β-[N-(N′,N′/-Dimethylaminoethane)-carbamoyl]Cholesterol (DC Cholesterol), dimethyldioctadecylammonium (DDA), 1,2-Dimyristoyl-3-TrimethylAmmoniumPropane (DMTAP), dipalmitoyl(C16:0)trimethyl ammonium propane (DPTAP), distearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), or a combination thereof.

19. The composition of claim Implementation 18, wherein the cationic lipid is 1,2-dioleoyloxy-3-(trimethylammonio)propane (DOTAP).

20. The composition of any of claims Implementation 1 -Implementation 19, wherein the hydrophobic surfactant is a sorbitan ester.

21. The composition of claim Implementation 20, wherein the sorbitan ester is a sorbitan monoester.

22. The composition of claim Implementation 21, wherein the sorbitan monoester is sorbitan monostearate.

23. The composition of claim Implementation 21, wherein the sorbitan monoester is sorbitan monooleate.

24. The composition of claim Implementation 20, wherein the sorbitan ester is a sorbitan triester.

25. The composition of claim Implementation 24, wherein the sorbitan triester is sorbitan trioleate or sorbitan tristearate.

26. The composition of any of claims Implementation 1 -Implementation 25, wherein the hydrophilic surfactant is a polysorbate.

27. The composition of claim Implementation 26, wherein the polysorbate is polysorbate 80.

28. The composition of any of claims Implementation 1 -Implementation 27, wherein the cake-forming excipient is a saccharide.

29. The composition of claim Implementation 28, wherein the saccharide is sucrose.

30. The composition of claim Implementation 28, wherein the saccharide is trehalose.

31. The composition of any of claims Implementation 28 -Implementation 30, wherein the saccharide is present at about 10-20% w/v.

32. The composition of claim Implementation 31, wherein the saccharide is present at about 20% w/v.

33. The composition of any of claims Implementation 1 -Implementation 32, wherein the liquid phase lipid is squalene or synthetic squalene, the solid phase lipid is trimyristin, the cationic lipid is DOTAP, the hydrophobic surfactant is sorbitan monostearate, the hydrophilic surfactant is polysorbate 80, and the cake-forming excipient is sucrose.

34. The composition of any one of claims Implementation 1 or Implementation 10-Implementation 33, wherein the z-average diameter of the NLC particles is from about 40 nm to about 60 nm.

35. The composition of any one of claims Implementation 2 -Implementation 33, wherein the z-average diameter of the NLC particles and bioactive agent is from about 90 nm to about 150 nm.

36. The composition of any one of claims Implementation 2 -Implementation 35, having a loading capacity for RNA of at least about 100 ng/μL RNA.

37. The composition of claim Implementation 36, having a loading capacity for RNA of at least about 200 ng/μL RNA.

38. The composition of any one of claims Implementation 2 -Implementation 37, having a nitrogen:phosphate (N:P) ratio of about 15.

39. The composition of any one of claims Implementation 1 -Implementation 38, comprising from about 0.2% to about 40% w/v liquid phase lipid, from about 0.1% to about 10% w/v solid phase lipid, from about 0.2% to about 10% w/v cationic lipid, from about 0.25% to about 15% w/v hydrophobic surfactant, from about 0.2% to about 15% w/v hydrophilic surfactant, and from about 15% to 25% w/v cake-forming excipient.

40. The composition of claim Implementation 39, about 3.75% w/v liquid phase lipid, about 0.24% w/v solid phase lipid, about 3% w/v cationic lipid, about 3.7% w/v sorbitan ester, about 3.7% w/v hydrophilic surfactant, and about 20% w/v cake-forming excipient.

41. The composition of any one of claims Implementation 39 -Implementation 40, wherein the cake-forming excipient is sucrose.

42. The composition of any one of claims Implementation 39 -Implementation 40, wherein the cake-forming excipient is trehalose.

43. The composition of any one of claims Implementation 1 -Implementation 42, wherein a hydrophilic surfactant to cationic lipid molar ratio is about 0.2 to about 1.5.

44. The composition of claim Implementation 43, wherein the hydrophilic surfactant to cationic lipid molar ratio is about 0.5 to about 1.

45. The composition of any one of claims Implementation 1 -Implementation 44, wherein an oil to surfactant molar ratio is about 0.05 to about 12.

46. The composition of claim Implementation 45, wherein the oil to surfactant molar ratio is about 0.5 to about 1.

47. The composition of any one of claims Implementation 1 -Implementation 46, wherein the composition is thermostable at about 25° C. for at least 6 months.

48. The composition of claim Implementation 47, wherein the composition is thermostable at about 25° C. for at least 8 months.

49. The composition of any one of claims Implementation 1 -Implementation 46, wherein the composition is thermostable at about 4° C. for at least 12 months.

50. The composition of claim Implementation 49, wherein the composition is thermostable at about 4° C. for at least 21 months.

51. The composition of any one of claims Implementation 47 -Implementation 50, wherein thermostability is determined by the cake maintaining size, structure, and color.

52. The composition of any one of claims Implementation 47 -Implementation 50, wherein thermostability is determined by assay of components of the oil-in-water emulsion following reconstitution.

53. The composition of any one of claims Implementation 47 -Implementation 50, wherein thermostability is determined by change in z-average diameter of less than 20%.

54. The composition of any one of claims Implementation 47 -Implementation 50, wherein thermostability is determined by RNA integrity.

55. A method of generating a thermostable, lyophilized composition for delivery of a bioactive agent to a cell, the method comprising:
generating NLC particles by mixing the solid phase lipid, the liquid phase lipid, the cationic lipid, and the hydrophobic surfactant to form an oil phase mixture; mixing the hydrophilic surfactant and an aqueous buffer to form an aqueous phase mixture; and mixing the oil phase mixture with the aqueous phase mixture;
mixing the NLC particles with a buffer containing the cake-forming excipient; and
lyophilizing the NLC particles with the buffer containing the cake-forming excipient wherein the composition is in the form of a cake and forms an oil-in-water emulsion upon reconstitution.

56. The method of claim Implementation 55, further comprising combining the NLC particles and buffer containing the cake-forming excipient with the bioactive agent such that the bioactive agent electrostatically complexes with the outer surface of the NLC particles.

57. The method of claim Implementation 56, wherein the bioactive agent is RNA and the NLC particles are combined with the bioactive agent at a nitrogen:phosphate (N/P) ratio of about 15.

58. The method of any of claims Implementation 55-Implementation 57, wherein the cake-forming excipient is sucrose.

59. The method of any of claims Implementation 55-Implementation 57, wherein the cake-forming excipient is trehalose.

60. The method of any of claims Implementation 58-Implementation 59, wherein the composition prior to lyophilization comprises about 10-20% w/v of the cake-forming excipient.

61. The method of claim Implementation 60, wherein the composition prior to lyophilization comprises about 20% w/v sucrose.

62. A method of stimulating an immune response in a subject comprising:
reconstituting the cake of any one of claims Implementation 1-Implementation 54 into an oil-in-water emulsion;
combining the oil-in-water emulsion with a bioactive agent; and
administering to the subject in an amount effective to stimulate the immune response in the subject.

63. A method of stimulating an immune response in a subject comprising:
reconstituting the cake of any one of claims Implementation 2-Implementation 54 into an oil-in-water emulsion; and
administering the emulsion to the subject in an amount effective to stimulate the immune response in the subject.

64. The method of claim Implementation 62 or Implementation 63, wherein the immune response is an antigen-specific immune response.

65. The method of claim Implementation 64, wherein the bioactive agent is RNA encoding the Zika pre-membrane (PrM) and envelope (E) proteins.

66. The method of claim Implementation 64, wherein the bioactive agent is RNA encoding the SARS-CoV-2 spike protein.

67. The method of any of claims Implementation 62-Implementation 66, wherein the subject is a mammal.

68. The method of any of claims Implementation 62-Implementation 66, wherein the oil-in-water emulsion is administered intramuscularly.

69. The method of any of claims Implementation 62-Implementation 66, wherein the oil-in-water emulsion is administered intranasally.
